SEQUENCE LISTING (129 sequences)

SEQ ID NO: 1 - DNA, 2318 bp, Homo sapiens
gcacagaagcgagtccgactgtgctcgctgctcagcgccgcacccggaagatgaggctcgccgtgggagccctgctggtctgcgccgtcctggggctgtgtctggctgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaaaatctcagaggtagggctgccaccaaggtgaagatgggaacgcagatgatccatgagtttgccctggtttcactggcccaagtggtttgtgctaaccacgtctgtcttcacagctctgtgttgccatgtgtgctgaacaaaaaataaaaattattattgattttatatttc

SEQ ID NO: 2 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 3 - protein, 679 aa, Homo sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 4 - protein, 30 aa, Homo sapiens
HAEGTFTSDVSSYLEGQAAKEFIAWLVKGR

SEQ ID NO: 5 - protein, 12 aa, Unknown
  neutrophil lactoferrin splice variant
EDCIALKGEADA

SEQ ID NO: 6 - protein, 31 aa, Homo sapiens
  misc_feature (31)..(31): Xaa can be any naturally occurring amino acid
HAEGTFTSDVSSYLEGQAAKEFIAWLVKGRX

SEQ ID NO: 7 - protein, 28 aa, Homo sapiens
HAEGTFTSDVSSYLEGQAAKEFIAWLVK

SEQ ID NO: 8 - protein, 29 aa, Homo sapiens
HAEGTFTSDVSSYLEGQAAKEFIAWLVKG

SEQ ID NO: 9 - protein, 31 aa, Artificial Sequence
  GLP-1 analog
XXEGTFTSDVSSYLXGQAAKXFIAWLVKGRX

SEQ ID NO: 10 - protein, 12 aa, Unknown
  linker peptide
PEAPTDPEAPTD

SEQ ID NO: 11 - DNA, 14425 bp, Artificial Sequence
  nucleotide sequence of pREX0585
gcggccgcccgtaatgcggtatcgtgaaagcgaaaaaaaaactaacagtagataagacagatagacagatagagatggacgagaaacagggggggagaaaaggggaaaagagaaggaaagaaagactcatctatcgcagataagacaatcaaccctcatggcgcctccaaccaccatccgcactagggaccaagcgctcgcaccgttagcaacgcttgactcacaaaccaactgccggctgaaagagcttgtgcaatgggagtgccaattcaaaggagccgaatacgtctgctcgccttttaagaggctttttgaacactgcattgcacccgacaaatcagccactaactacgaggtcacggacacatataccaatagttaaaaattacatatactctatatagcacagtagtgtgataaataaaaaattttgccaagacttttttaaactgcacccgacagatcaggtctgtgcctactatgcacttatgcccggggtcccgggaggagaaaaaacgagggctgggaaatgtccgtggactttaaacgctccgggttagcagagtagcagggctttcggctttggaaatttaggtgacttgttgaaaaagcaaaatttgggctcagtaatgccactgcagtggcttatcacgccaggactgcgggagtggcgggggcaaacacacccgcgataaagagcgcgatgaatataaaagggggccaatgttacgtcccgttatattggagttcttcccatacaaacttaagagtccaattagcttcatcgccaataaaaaaacaagctaaacctaattctaacaagcaaagatgaggctcgccgtgggagccctgctggtctgcgccgtcctggggctgtgtctggcgcatggtgaaggtacttttacttctgatgtttcttcttatttggaaggtcaagctgctaaagaatttattgcttggttggttgctggtaggggtccagaagctccaactgatccagaagctccaactgatgtacctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataaggctgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctctgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtactaggaaacctgtggaggagtatgcgaactgccacctggccagagccccgaatcacgctgtggtcacacggaaagataaggaagcatgcgtccacaagatattacgtcaacagcagcacctatttggaagcaacgtagctgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtcgaccttaataagcttaattcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatcgttcttccacacggatcgcggccgctagtcgagtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgaggggatcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagggccagtgaattccctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagtgcgaagtagtgatcaggtattgctgttatctgatgagtatacgttgtcctggccacggcagaagcacgcttatcgctccaatttcccacaacattagtcaactccgttaggcccttcattgaaagaaatgaggtcatcaaatgtcttccaatgtgagattttgggccattttttatagcaaagattgaataaggcgcatttttcttcaaagctttattgtacgatctgactaagttatcttttaataattggtattcctgtttattgcttgaagaattgccggtcctatttactcgttttaggactggttcagaattcctcaaaaattcatccaaatatacaagtggatcgatcctaccccttgcgctaaagaagtatatgtgcctactaacgcttgtctttgtctctgtcactaaacactggattattactcccagatacttattttggactaatttaaatgatttcggatcaacgttcttaatatcgctgaatcttccacaattgatgaaagtagctaggaagaggaattggtataaagtttttgtttttgtaaatctcgaagtatactcaaacgaatttagtattttctcagtgatctcccagatgctttcaccctcacttagaagtgctttaagcatttttttactgtggctatttcccttatctgcttcttccgatgattcgaactgtaattgcaaactacttacaatatcagtgatatcagattgatgtttttgtccatagtaaggaataattgtaaattcccaagcaggaatcaatttctttaatgaggcttccagaattgttgctttttgcgtcttgtatttaaactggagtgatttattgacaatatcgaaactcagcgaattgcttatgatagtattatagctcatgaatgtggctctcttgattgctgttccgttatgtgtaatcatccaacataaataggttagttcagcagcacataatgctattttctcacctgaaggtctttcaaacctttccacaaactgacgaacaagcaccttaggtggtgttttacataatataccaaattgtggcatgcttagcgccgatcttgtgtgcaattgatatctagtttcaactactctatttatcttgtatcttgcagtattcaaacacgctaactcgaaaaactaactttaattgtcctgtttgtctcgcgttctttcgaaaaatgcaccggccgcgcattatttgtactgcgaaaataattggtactgcggtatcttcatttcatattttaaaaatgcacctttgctgcttttccttaatttttagacggcccgcaggttcgttttgcggtactatcttgtgataaaaagttgttttgacatgtgatctgcacagattttataatgtaataagcaagaatacattatcaaacgaacaatactggtaaaagaaaaccaaaatggacgacattgaaacagccaagaatctgacggtaaaagcacgtacagcttatagcgtctgggatgtatgtcggctgtttattgaaatgattgctcctgatgtagatattgatatagagagtaaacgtaagtctgatgagctactctttccaggatatgtcataaggcccatggaatctctcacaaccggtaggccgtatggtcttgattctagcgcagaagattccagcgtatcttctgactccagtgctgaggtaattttgcctgctgcgaagatggttaaggaaaggtttgattcgattggaaatggtatgctctcttcacaagaagcaagtcaggctgccatagatttgatgctacagaataacaagctgttagacaatagaaagcaactatacaaatctattgctataataataggaagattgcccgagaaagacaagaagagagctaccgaaatgctcatgagaaaaatggattgtacacagttattagtcccaccagctccaacggaagaagatgttatgaagctcgtaagcgtcgttacccaattgcttactttagttccaccagatcgtcaagctgctttaataggtgatttattcatcccggaatctctaaaggatatattcaatagtttcaatgaactggcggcagagaatcgtttacagcaaaaaaagagtgagttggaaggaaggactgaagtgaaccatgctaatacaaatgaagaagttccctccaggcgaacaagaagtagagacacaaatgcaagaggagcatataaattacaaaacaccatcactgagggccctaaagcggttcccacgaaaaaaaggagagtagcaacgagggtaaggggcagaaaatcacgtaatacttctagggtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacatcaacaggcttccaatgctcttcaaattttactgtcaagtagacccatacggctgtaatatgctgctcttcataatgtaagcttatctttatcgaatcgtgtgaaaaactactaccgcgataaacctttacggttccctgagattgaattagttcctttagtatatgatacaagacacttttgaactttgtacgacgaattttgaggttcgccatcctctggctatttccaattatcctgtcggctattatctccgcctcagtttgatcttccgcttcagactgccatttttcacataatgaatctatttcaccccacaatccttcatccgcctccgcatcttgttccgttaaactattgacttcatgttgtacattgtttagttcacgagaagggtcctcttcaggcggtagctcctgatctcctatatgacctttatcctgttctctttccacaaacttagaaatgtattcatgaattatggagcacctaataacattcttcaaggcggagaagtttgggccagatgcccaatatgcttgacatgaaaacgtgagaatgaatttagtattattgtgatattctgaggcaattttattataatctcgaagataagagaagaatgcagtgacctttgtattgacaaatggagattccatgtatctaaaaaatacgcctttaggccttctgataccctttcccctgcggtttagcgtgccttttacattaatatctaaaccctctccgatggtggcctttaactgactaataaatgcaaccgatataaactgtgataattctgggtgatttatgattcgatcgacaattgtattgtacactagtgcaggatcaggccaatccagttctttttcaattaccggtgtgtcgtctgtattcagtacatgtccaacaaatgcaaatgctaacgttttgtatttcttataattgtcaggaactggaaaagtcccccttgtcgtctcgattacacacctactttcatcgtacaccataggttggaagtgctgcataatacattgcttaatacaagcaagcagtctctcgccattcatatttcagttattttccattacagctgatgtcattgtatatcagcgctgtaaaaatctatctgttacagaaggttttcgcggtttttataaacaaaactttcgttacgaaatcgagcaatcaccccagctgcgtatttggaaattcgggaaaaagtagagcaacgcgagttgcattttttacaccataatgcatgattaacttcgagaagggattaaggctaatttcactagtatgtttcaaaaacctcaatctgtccattgaatgccttataaaacagctatagattgcatagaagagttagctactcaatgctttttgtcaaagcttactgatgatgatgtgtctactttcaggcgggtctgtagtaaggagaatgacattataaagctggcacttagaattccacggactatagactatactagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttaacgacgt

SEQ ID NO: 12 - protein, 722 aa, Artificial Sequence
  GLP-1-tf fusion protein
HGEGTFTSDVSSYLEGQAAKEFIAWLVAGRGPEAPTDPEAPTDVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKADNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVADCSGNFCLFRSETKDLLFRDDTVCLAKLHDRNTYEKYLGEEYVKAVGNLRKCSTSSLLEACTFRRP

SEQ ID NO: 13 - protein, 6 aa, Artificial Sequence
  non-helical linker peptide
PEAPTD

SEQ ID NO: 14 - protein, 18 aa, Artificial Sequence
  non-helical linker peptide
PEAPTDPEAPTDPEAPTD

SEQ ID NO: 15 - protein, 8 aa, Artificial Sequence
  linker peptide
SSGAPPPS

SEQ ID NO: 16 - protein, 12 aa, Artificial Sequence
  linker peptide
SGGGSGGGSGGG

SEQ ID NO: 17 - protein, 12 aa, Artificial Sequence
  non-helical linker peptide
PPPPPPPPPPPP

SEQ ID NO: 18 - protein, 12 aa, Artificial Sequence
  non-helical linker peptide
GEAPTDPEAPTD

SEQ ID NO: 19 - protein, 12 aa, Artificial Sequence
  non-helical linker peptide
PEAGTDPEAPTD

SEQ ID NO: 20 - protein, 12 aa, Artificial Sequence
  non-helical linker peptide
PEAPTDGEAPTD

SEQ ID NO: 21 - protein, 12 aa, Artificial Sequence
  non-helical linker peptide
PEAPTDPEAGTD

SEQ ID NO: 22 - protein, 12 aa, Artificial Sequence
  non-helical linker peptide
PQAPTNPQAPTN

SEQ ID NO: 23 - protein, 12 aa, Artificial Sequence
  non-helical linker peptide
PEAPEAPEAPEA

SEQ ID NO: 24 - DNA, 25 bp, Artificial sequence
  designing linker P0273
ctaggtctctagagaaaaggcatgc

SEQ ID NO: 25 - DNA, 61 bp, Artificial Sequence
  designing linker P0274
aaataagaagaaacatcagaagtaaaagtaccttcagcatgccttttctctagagacctag

SEQ ID NO: 26 - DNA, 73 bp, Artificial Sequence
  designing linker P0275
tgaaggtacttttacttctgatgtttcttcttatttggaaggtcaagctgctaaagaatttattgcttggttg

SEQ ID NO: 27 - DNA, 68 bp, Artificial Sequence
  designing linker P0276
agaaagaaccatcagcatgcctacctttaaccaaccaagcaataaattctttagcagcttgaccttcc

SEQ ID NO: 28 - DNA, 62 bp, Artificial Sequence
  designing linker P0277
gttaaaggtaggcatgctgatggttctttctctgatgagatgaacaccattcttgataatct

SEQ ID NO: 29 - DNA, 67 bp, Artificial Sequence
  designing linker P0278
gtctgaatcaaccagtttataaagtccctggcggcaagattatcaagaatggtgttcatctcatcag

SEQ ID NO: 30 - DNA, 61 bp, Artificial Sequence
  designing linker P0279
tgccgccagggactttataaactggttgattcagaccaaaatcactgacagggtacctgat

SEQ ID NO: 31 - DNA, 25 bp, Artificial Sequence
  designing linker P0280
atcaggtaccctgtcagtgattttg

SEQ ID NO: 32 - DNA, 21 bp, Artificial Sequence
  PCR primer P0012
catgatcttggcgatgcagtc

SEQ ID NO: 33 - DNA, 24 bp, Artificial Sequence
  PCR primer P0025
agcggataacaatttcacacagga

SEQ ID NO: 34 - DNA, 39 bp, Artificial Sequence
  mutagenic primer P0538
gttggttaatggtaggggttcttctgtacctgataaaac

SEQ ID NO: 35 - DNA, 39 bp, Artificial Sequence
  mutagenic primer P0539
gttttatcaggtacagaagaacccctaccattaaccaac

SEQ ID NO: 36 - DNA, 42 bp, Artificial Sequence
  mutagenic primer P0540
gttggttaatggtaggggttcttctggtgtacctgataaaac

SEQ ID NO: 37 - DNA, 42 bp, Artificial Sequence
  mutagenic primer P0541
gttttatcaggtacaccagaagaacccctaccattaaccaac

SEQ ID NO: 38 - DNA, 24 bp, Artificial Sequence
  mutagenic primer P0025
agcggataacaatttcacacagga

SEQ ID NO: 39 - DNA, 30 bp, Artificial Sequence
  mutagenic primer P0550
aatggtaggggtccagaagctgtacctgat

SEQ ID NO: 40 - DNA, 30 bp, Artificial Sequence
  mutagenic primer P0551
atcaggtacagcttctggacccctaccatt

SEQ ID NO: 41 - DNA, 40 bp, Artificial Sequence
  mutagenic primer P0552
atggtaggggtccagaagctccaactgatgtacctgataa

SEQ ID NO: 42 - DNA, 40 bp, Artificial Sequence
  mutagenic primer P0553
ttatcaggtacatcagttggagcttctggacccctaccat

SEQ ID NO: 43 - DNA, 28 bp, Artificial Sequence
  mutagenic primer P0554
gtaggggtccagaagctcctgataaaac

SEQ ID NO: 44 - DNA, 28 bp, Artificial Sequence
  mutagenic primer P0555
gttttatcaggagcttctggacccctac

SEQ ID NO: 45 - DNA, 50 bp, Artificial Sequence
  mutagenic primer P0661
ttggttgctggtaggggtccagaagctccaactgatgtacctgataaaac

SEQ ID NO: 46 - DNA, 50 bp, Artificial Sequence
  mutagenic primer P0662
gttttatcaggtacatcagttggagcttctggacccctaccagcaaccaa

SEQ ID NO: 47 - DNA, 70 bp, Artificial Sequence
  mutagenic primer P0663
ttggttgctggtaggggtccagaagctccaactgatccagaagctccaactgatgtacctgataaaactg

SEQ ID NO: 48 - DNA, 70 bp, Artificial Sequence
  mutagenic primer P0664
cagttttatcaggtacatcagttggagcttctggatcagttggagcttctggacccctaccagcaaccaa

SEQ ID NO: 49 - DNA, 92 bp, Artificial Sequence
  mutagenic primer P0665
ggttggttgctggtaggggtccagaagctccaactgatccagaagctccaactgatccagaagctccaactgatgtacctgataaaactgtg

SEQ ID NO: 50 - DNA, 92 bp, Artificial Sequence
  mutagenic primer P0666
cacagttttatcaggtacatcagttggagcttctggatcagttggagcttctggatcagttggagcttctggacccctaccagcaaccaacc

SEQ ID NO: 51 - DNA, 110 bp, Artificial Sequence
  mutagenic primer P0722
ctcacagttttatcaggtacatcagttggagcttctggatcagttggagcttctggatcagttggagcttctggatcagttggagcttctggacccctaccagcaaccaa

SEQ ID NO: 52 - DNA, 84 bp, Artificial Sequence
  primer P0676
atggtggcgaagtatgagttttatccgacgattttggttcaacacccctaccagcaaccaaccaagcaataaattctttagcag

SEQ ID NO: 53 - DNA, 87 bp, Artificial Sequence
  primer P0677
gtcggataaaactcatacttcgccaccatcgccagctccagaattgttgggtggtccatcggtacctgataaaactgtgagatggtg

SEQ ID NO: 54 - DNA, 101 bp, Artificial Sequence
  primer P0678
cgaagtatgagttttatccgacgattttggttcaaccgatggtggtggagcacccgacgaacccctaccagcaaccaaccaagcaataaattctttagcag

SEQ ID NO: 55 - DNA, 98 bp, Artificial Sequence
  primer P0679
gttgaaccaaaatcgtcggataaaactcatacttcgccaccatcgccagctccagaattgttgggtggtccatcggtacctgataaaactgtgagatg

SEQ ID NO: 56 - DNA, 103 bp, Artificial Sequence
  primer P0680
gatggtggcgaagtatgagttttatccgacgattttggttcaacatcagttggagcttctggacccctaccagcaaccaaccaagcaataaattctttagcag

SEQ ID NO: 57 - DNA, 94 bp, Artificial Sequence
  primer P0681
caaaatcgtcggataaaactcatacttcgccaccatcgccagctccagaattgttgggtggtccatcggtacctgataaaactgtgagatggtg

SEQ ID NO: 58 - DNA, 78 bp, Artificial Sequence
  primer P0719
ctttagcagcttgttcttccaaataagaagaaacatcagaagtaaaagtaccttcaccatgcgccagacacagcccca

SEQ ID NO: 59 - DNA, 51 bp, Artificial Sequence
  primer P0720
gtacttttacttctgatgtttcttcttatttggaagaacaagctgctaaag

SEQ ID NO: 60 - DNA, 21 bp, Artificial Sequence
  primer P0723
ccgttatattggagttcttcc

SEQ ID NO: 61 - DNA, 22 bp, Artificial Sequence
  primer P0724
actgctttctcaagaggtttac

SEQ ID NO: 62 - DNA, 43 bp, Artificial Sequence
  primer P0745
gttgaaccaaaatcgtcggataaaactcatacttcgccaccat

SEQ ID NO: 63 - protein, 31 aa, Artificial Sequence
  GLP-1 analog
HGEGTFTSDVSSYLEGQAAKEFIAWLVAGRG

SEQ ID NO: 64 - protein, 19 aa, Unknown
  nL leader sequence
MRLAVGALLVCAVLGLCLA

SEQ ID NO: 65 - DNA, 2229 bp, Artificial Sequence
  fusion protein nL GLP-1 (7-37;A8G,K34A) (PEAPTD)2 mTf
atgaggctcgccgtgggagccctgctggtctgcgccgtcctggggctgtgtctggcgcatggtgaaggtacttttacttctgatgtttcttcttatttggaaggtcaagctgctaaagaatttattgcttggttggttgctggtaggggtccagaagctccaactgatccagaagctccaactgatgtacctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataaggctgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctctgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtactaggaaacctgtggaggagtatgcgaactgccacctggccagagccccgaatcacgctgtggtcacacggaaagataaggaagcatgcgtccacaagatattacgtcaacagcagcacctatttggaagcaacgtagctgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtcgaccttaataa

SEQ ID NO: 66 - protein, 741 aa, Artificial Sequence
  fusion protein nL GLP-1 (7-37;A8G,K34A) (PEAPTD)2 mTf
MRLAVGALLVCAVLGLCLAHGEGTFTSDVSSYLEGQAAKEFIAWLVAGRGPEAPTDPEAPTDVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKADNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVADCSGNFCLFRSETKDLLFRDDTVCLAKLHDRNTYEKYLGEEYVKAVGNLRKCSTSSLLEACTFRRP

SEQ ID NO: 67 - DNA, 118 bp, Artificial Sequence
  nucleotide sequence of nl-GLP-1 (7-37) mTF fusion peptide
aggtctctagagaaaaggcatgctgaaggtacttttacttctgatgtttcttcttatttggaaggtcaagctgctaaagaatttattgcttggttggttaaaggtagggtacctgata

SEQ ID NO: 68 - DNA, 118 bp, Artificial Sequence
  reverse complement of nucleotide sequence of nl-GLP-1 (7-37) mTF fusion peptide
tatcaggtaccctacctttaaccaaccaagcaataaattctttagcagcttgaccttccaaataagaagaaacatcagaagtaaaagtaccttcagcatgccttttctctagagacct

SEQ ID NO: 69 - DNA, 90 bp, Artificial Sequence
  yeast optimized GLP-1(7-36)
catgctgaaggtacttttacttctgatgtttcttcttatttggaaggtcaagctgctaaagaatttattgcttggttggttaaaggtaga

SEQ ID NO: 70 - DNA, 21 bp, Artificial Sequence
  mutagenic primer P0012
catgatcttggcgatgcagtc

SEQ ID NO: 71 - protein, 31 aa, Artificial Sequence
  GLP-1 peptide variant
HAEGTFTSDVSSYLEGQAAKEFIAWLVKGRG

SEQ ID NO: 72 - protein, 31 aa, Artificial Sequence
  GLP-1 peptide variant
HHAEGTFTSDVSSYLEGQAAKEFIAWLVKGR

SEQ ID NO: 73 - protein, 30 aa, Artificial Sequence
  GLP-1 peptide variant
HSEGTFTSDVSSYLEGQAAKEFIAWLVXGR

SEQ ID NO: 74 - protein, 34 aa, Artificial Sequence
  linker peptide
HADGSFSDEMNTILDNLAARDFINWLIQTKITDR

SEQ ID NO: 75 - protein, 67 aa, Artificial Sequence
  N-terminal portion of fusion protein H GLP-1(7-36)K34Q-GLP-2(2-34)K30A-mTf
HHAEGTFTSDVSSYLEGQAAKEFIAWLVQGRADGSFSDEMNTILDNLAARDFINWLIQTAITDRVSD

SEQ ID NO: 76 - DNA, 110 bp, Artificial Sequence
  mutagenic primer P0721
ttggttgctggtaggggtccagaagctccaactgatccagaagctccaactgatccagaagctccaactgatccagaagctccaactgatgtacctgataaaactgtgag

SEQ ID NO: 77 - protein, 4 aa, Artificial Sequence
  dipeptidyl-peptidase resistant N-terminal end for GLP-1 analogs
HHAE

SEQ ID NO: 78 - protein, 4 aa, Artificial Sequence
  dipeptidyl-peptidase resistant N-terminal end for GLP-1 analogs
HHGE

SEQ ID NO: 79 - protein, 4 aa, Artificial Sequence
  dipeptidyl-peptidase resistant N-terminal end for GLP-1 analogs
HHSE

SEQ ID NO: 80 - protein, 4 aa, Artificial Sequence
  dipeptidyl-peptidase resistant N-terminal end for GLP-1 analogs
GHAE

SEQ ID NO: 81 - protein, 4 aa, Artificial Sequence
  dipeptidyl-peptidase resistant N-terminal end for GLP-1 analogs
GHGE

SEQ ID NO: 82 - protein, 4 aa, Artificial Sequence
  dipeptidyl-peptidase resistant N-terminal end for GLP-1 analogs
GHSE

SEQ ID NO: 83 - protein, 31 aa, Artificial Sequence
  substituted GLP-1
HAEGTFTSDVSSYLEGQAAXEFIAWLVXGXG

SEQ ID NO: 84 - protein, 31 aa, Artificial Sequence
  substituted GLP-1
HAEGTFTSDVSSYLEGQAAKEFIAXLVKGRG

SEQ ID NO: 85 - protein, 31 aa, Artificial Sequence
  substituted GLP-1
HAEGTFTSDXSXYLXXXXAXEFIAWLVKGRG

SEQ ID NO: 86 - protein, 31 aa, Artificial Sequence
  substituted GLP-1
HXXXTFTSXVSSYLEGQAAKEFIAWLVKGRG

SEQ ID NO: 87 - protein, 31 aa, Artificial Sequence
  substituted GLP-1
XAEGTFTSDVSSYLEGQAAKEFIAWLVKGRG

SEQ ID NO: 88 - protein, 15 aa, Artificial Sequence
  IgG hinge amino acid sequence
EPKSCDKTHTCPPCP

SEQ ID NO: 89 - protein, 25 aa, Artificial Sequence
  linker peptide
VEPKSSDKTHTSPPSPAPELLGGPS

SEQ ID NO: 90 - protein, 34 aa, Artificial Sequence; identical to SEQ ID NO: 74
  linker peptide

SEQ ID NO: 91 - protein, 33 aa, Artificial Sequence
  linker peptide
ADGSFSDEMNTILDNLAARDFINWLIQTKITDR

SEQ ID NO: 92 - DNA, 73 bp, Artificial Sequence
  primer P0281
ctagagaaaaggcatgctgaaggtacttttacttctgatgtttcttcttatttggaaggtcaagctgctaaag

SEQ ID NO: 93 - DNA, 70 bp, Artificial Sequence
  primer P0282
aatttattgcttggttggttaaaggtaggtctggtggtggttctggtggtggttctggtggtggtggtac

SEQ ID NO: 94 - DNA, 81 bp, Artificial Sequence
  primer P0283
caccaccaccagaaccaccaccagaaccaccaccagacctacctttaaccaaccaagcaataaattctttagcagcttgac

SEQ ID NO: 95 - DNA, 54 bp, Artificial Sequence
  primer P0284
cttccaaataagaagaaacatcagaagtaaaagtaccttcagcatgccttttct

SEQ ID NO: 96 - protein, 34 aa, Artificial Sequence
  GLP-1 analog
HADGSFSDEMNTILDNLATRDFINWLIQTKITDR

SEQ ID NO: 97 - protein, 19 aa, Bos taurus
MRPAVRALLACAVLGLCLA

SEQ ID NO: 98 - protein, 19 aa, Equus caballus
MRLAIRALLACAVLGLCLA

SEQ ID NO: 99 - protein, 19 aa, Mus musculus
MRLTVGALLACAALGLCLA

SEQ ID NO: 100 - protein, 19 aa, Oryctolagus cuniculus
MRLAAGALLACAALGLCLA

SEQ ID NO: 101 - protein, 19 aa, Rattus norvegicus
MRFAVGALLACAALGLCLA

SEQ ID NO: 102 - protein, 19 aa, Bos taurus
MKLFVPALLSLGALGLCLA

SEQ ID NO: 103 - protein, 19 aa, Bubalus bubalis
MKLFVPALLSLGALGLCLA

SEQ ID NO: 104 - protein, 19 aa, Camelus dromedaries
MKLFFPALLSLGALGLCLA

SEQ ID NO: 105 - protein, 19 aa, Capra hircus
MKLFVPALLSLGALGLCLA

SEQ ID NO: 106 - protein, 6 aa, Equus caballus
LGLCLA

SEQ ID NO: 107 - protein, 19 aa, Mus musculus
MRLLIPSLIFLEALGLCLA

SEQ ID NO: 108 - protein, 19 aa, Sus scrofa
MKLFIPALLFLGTLGLCLA

SEQ ID NO: 109 - protein, 19 aa, Sus scrofa
MRLAFCVLLCAGSLGLCLA

SEQ ID NO: 110 - protein, 19 aa, Homo sapiens
MRGPSGALWLLLALRTVLG

SEQ ID NO: 111 - protein, 19 aa, Mus musculus
MRLLSVTFWLLLSLRTVVC

SEQ ID NO: 112 - protein, 19 aa, Oryctolagus cuniculus
MRCRSAAMWIFLALRTALG

SEQ ID NO: 113 - protein, 6 aa, Artificial Sequence
  pro-peptide sequence to ensure efficient removal of the signal sequence
RSLDKR

SEQ ID NO: 114 - protein, 6 aa, Artificial Sequence
  pro-peptide sequence to ensure efficient removal of the signal sequence
RSLDRR

SEQ ID NO: 115 - protein, 6 aa, Artificial Sequence
  pro-peptide sequence to ensure efficient removal of the signal sequence
RSLEKR

SEQ ID NO: 116 - protein, 6 aa, Artificial Sequence
  pro-peptide sequence to ensure efficient removal of the signal sequence
RSLERR

SEQ ID NO: 117 - protein, 25 aa, Artificial Sequence
  synthetic modified human IgG hinge
VEPKSADKTHTAPPAPAPELLGGPS

SEQ ID NO: 118 - protein, 21 aa, Artificial Sequence
  peptide linker
PEAPTDEPKSCDKTHTCPPCP

SEQ ID NO: 119 - protein, 31 aa, Artificial Sequence
  peptide linker
PEAPTDVEPKSSDKTHTSPPSPAPELLGGPS

SEQ ID NO: 120 - protein, 31 aa, Artificial Sequence
  peptide linker
PEAPTDVEPKSADKTHTAPPAPAPELLGGPS

SEQ ID NO: 121 - protein, 21 aa, Artificial Sequence
  peptide linker
EPKSCDKTHTCPPCPPEAPTD

SEQ ID NO: 122 - protein, 31 aa, Artificial Sequence
  peptide linker
VEPKSSDKTHTSPPSPAPELLGGPSPEAPTD

SEQ ID NO: 123 - protein, 31 aa, Artificial Sequence
  peptide linker
VEPKSADKTHTAPPAPAPELLGGPSPEAPTD

SEQ ID NO: 124 - protein, 25 aa, Artificial Sequence
  peptide linker
VEPKSCDKTHTCPPCPAPELLGGPS

SEQ ID NO: 125 - protein, 25 aa, Artificial Sequence
  peptide linker
VEPKAADKTHTAPPAPAPELLGGPA

SEQ ID NO: 126 - protein, 31 aa, Artificial Sequence
  peptide linker
PEAPTDVEPKSCDKTHTCPPCPAPELLGGPS

SEQ ID NO: 127 - protein, 31 aa, Artificial Sequence
  peptide linker
VEPKSCDKTHTCPPCPAPELLGGPSPEAPTD

SEQ ID NO: 128 - protein, 31 aa, Artificial Sequence
  peptide linker
PEAPTDVEPKAADKTHTAPPAPAPELLGGPA

SEQ ID NO: 129 - protein, 31 aa, Artificial Sequence
  peptide linker
VEPKAADKTHTAPPAPAPELLGGPAPEAPTD

(19) United States
(12) Patent Application Publication
(10) Pub. No.: US 2006/0205037 A1
(43) Pub. Date: Sep. 14, 2006

(54) Modified transferrin fusion proteins

(76) Inventors: Homayoun Sadeghi, King of Prussia, PA (US); Andrew J. Turner, King of Prussia, PA (US); Christopher P. Prior, King of Prussia, PA (US); David J. Ballance, King of Prussia, PA (US)

Correspondence Address: COOLEY GODWARD LLP, THE BROWN BUILDING - 875 15TH STREET, NW, SUITE 800, WASHINGTON, DC, 20005-2221, US

(21) Appl. No.: 11/367,692

(22) Filed: Mar. 6, 2006

Related U.S. Application Data
(60) Provisional application No. 60/658,140, filed on Mar. 4, 2005.
(60) Provisional application No. 60/663,757, filed on Mar. 22, 2005.

(30) Foreign Application Priority Data
Aug. 28, 2003 (WO) PCT/US03/26818

(51) Int. Cl. C07K 14/605; C07H 21/04; C12P 21/04; A61K 38/26; A61K 38/40

(52) U.S. Cl. 435/69.7; 435/320.1; 435/325; 530/399; 536/23.5; 514/12

(57) ABSTRACT

Modified fusion proteins of a transferrini moiety, a GLP-1 moiety and a linker moiety, with increased productivity, bioactivity and serum half-life are disclosed. Preferred fusion proteins include those modified so that the transferrin moiety exhibits no or reduced glycosylation. The fusion proteins of the invention are useful for the treatment of Type 2 diabetes, Type 1 diabetes, obesity, congestive heart failure, and non-fatty liver disease.

### RELATED APPLICATIONS

This application claims the benefit of U.S. Provisional Application Ser. Nos. 60/658,140, filed Mar. 4, 2005 and 60/663,757, filed Mar. 22, 2005, both of which are herein incorporated by reference in their entirety for all purposes.

This application is related to but does not claim the benefit of International Application PCT/US03/26818, filed Aug. 28, 2003, which claims the benefit of U.S. application Ser. No. 10/378,094, filed Mar. 4, 2003, and U.S. application Ser. No. 10/231,494, filed Aug. 30, 2002, which claims the benefit of U.S. Provisional Application 60/315,745, filed Aug. 30, 2001 and U.S. Provisional Application 60/334,059, filed Nov. 30, 2001, all of which are herein incorporated by reference in their entirety. This application is also related to but does not claim the benefit of U.S. Provisional Application 60/406,977, filed Aug. 30, 2002, which is herein incorporated by reference in its entirety.

### FIELD OF THE INVENTION

The present invention relates to a GLP-1 and transferrin fusion protein and use thereof for the treatment of diseases associated with elevated glucose serum levels such as type II diabetes. The fusion protein of the invention can also be used to treat other diseases known to benefit from treatment with GLP-1 such as obesity, type I diabetes, congestive heart failure and non-alcoholic, non-fatty liver disease.

### BACKGROUND OF THE INVENTION

Therapeutic proteins or peptides in their native state or when recombinantly produced are typically labile molecules exhibiting short periods of serum stability or short in Vivo circulatory half-lives. In addition, these molecules are often extremely labile when formulated, particularly when formulated in aqueous solutions for diagnostic and therapeutic purposes.

Few practical solutions exist to extend or promote the stability in vivo or in vitro of proteinaceous therapeutic molecules. Polyethylene glycol (PEG) is a substance that can be attached to a protein, resulting in longer-acting, sustained activity of the protein. If the activity of a protein is prolonged by the attachment to PEG, the frequency that the protein needs to be administered may be decreased. PEG attachment, however, often decreases or destroys the protein's therapeutic activity. While in some instance PEG attachment can reduce immunogenicity of the protein, in other instances it may increase immunogenicity.

Therapeutic proteins or peptides have also been stabilized by fusion to a protein capable of extending the in vivo circulatory half-life of the therapeutic protein. For instance, therapeutic proteins fused to albumin or to antibody fragments may exhibit extended in vivo circulatory half-life when compared to the therapeutic protein in the unfused state. See U.S. Pat. Nos. 5,876,969 and 5,766,883.

Another serum protein, glycosylated human transferrin (Tf) has also been used to make fusions with therapeutic proteins to target delivery to the interior of cells or to carry agents across the blood-brain barrier. These fusion proteins comprising glycosylated human Tf have been used to target nerve growth factor (NGF) or ciliary neurotrophic factor (CNTF) across the blood-brain barrier by fusing full-length Tf to the agent. See U.S. Pat. Nos. 5,672,683 and 5977,307. In these fusion proteins, the Tf portion of the molecule is glycosylated and binds to two atoms of iron, which is required for Tf binding to its receptor on a cell and, according to the inventors of these patents, to target delivery of the NGF or CNTF moiety across the blood-brain barrier. Transferrin fusion proteins have also been produced by inserting an HIV-1 protease target sequence into surface exposed loops of glycosylated transferrin to investigate the ability to produce another form of Tf fusion for targeted delivery to the inside of a cell via the Tf receptor (Ali et al. (1999) J. Biol. Chem. 274(34):24066-24073).

Serum transferrin (Tf) is a monomeric glycoprotein with a molecular weight of 80,000 daltons that binds iron in the circulation and transports it to various tissues via the transferrin receptor (TfR) (Aisen et al. (1980) Ann. Rev. Biochem. 49: 357-393; MacGillivray et al. (1981) J. Biol. Chem. 258: 3543-3553. U.S. Pat. No. 5,026,651). Tf is one of the most common serum molecules, comprising up to about 5-10% of total serum proteins. Carbohydrate deficient transferrin occurs in elevated levels in the blood of alcoholic individuals and exhibits a longer half life (approximately 14-17 days) than that of glycosylated transferrin (approximately 7-10 days). See van Eijk et al. (1983) Clin. Chim. Acta 132:167-171, Stibler (1991) Clin. Chem. 37:2029-2037 (1991), Arndt (2001) Clin. Chem. 47(1):13-27 and Stibler et al. in “Carbohydrate-deficient consumption”, Advances in the Biosciences, (Ed Nordmann et al.), Pergamon, 1988, Vol. 71, pages 353-357).

The structure of Tf has been well characterized and the mechanisms of receptor binding, iron binding and release and carbonate ion binding have been elucidated (U.S. Pat. Nos. 5,026,651, 5,986,067 and MacGillivray et al. (1983) J. Biol. Chem. 258(6):3543-3546).

Transferrin and antibodies that bind the transferrin receptor have also been used to deliver or carry toxic agents to tumor cells as cancer therapy (Baselga and Mendelsohn, 1994), and transferrin has been used as a non-viral gene therapy vector to deliver DNA to cells (Frank et al., 1994; Wagner et al., 1992). The ability to deliver proteins to the central nervous system (CNS) using the transferrin receptor as the entry point has been demonstrated with several proteins and peptides including CD4 (Walus et al., 1996), brain derived neurotrophic factor (Pardridge et al., 1994), glial derived neurotrophic factor (Albeck et al.), a vasointestinal peptide analogue (Bickel et al., 1993), a beta-amyloid peptide (Saito et al., 1995), and an antisense oligonucleotide (Pardridge et al., 1995).

GLP-1 is a 30/31 amino acid therapeutic peptide derived from post-translational processing of the proglucagon gene product in intestinal enteroendocrine L cells. Infusion of GLP-1 to humans suffering from type 2 diabetes stimulates insulin secretion and lowers blood glucose in a glucose-dependent manner (Nauck, M., 2004, Horm. Metab. Res. 36: 852-858: Vilsboll et al., 2004, Diabetologia. 47: 357-366; and Zander et al., 2002, Lancet. 359: 824-830). GLP1 has also been found to promote satiety and inhibit gastric emptying (Zander et al., 2002, Lancet. 359: 824-830; Gutzwiller et al., 1999, Gut. 44: 81-86; Meier et al., 2002, Eur. J. Pharmacol. 440: 269-279; and Flint et al., 2001, Int. J. Obes. Relat. Metab. Disord. 25: 781-792). Infusions of GLP1 cause an increase in insulin biosynthiesis and an increase in β-cell proliferation and β-cell mass in islets of Langerhans of rodents (Perfetti et al., 2000, Endocrinology. 141: 4600-4605; Wang et al., 1997, J. Clin. Invest. 99: 2883-2889; and Stoffers et al., 2000, Diabetes. 49: 741-748).

Full-length, active GLP-1 has a short circulatling t_{1/2} of 1-2 minutes because of rapid enzymatic inactivation by dipeptidyl peptidase IV (DPPIV) due to cleavage betweenl alanine and glutamic acid in the second and third positions, respectively, of the N-terminus of the peptide (Kieffer et al., 1995, Endocrinology. 136: 3585-3596). The remaining fragment of GLP-1 comprising 28/29 amino acids is not insulinotropic and is further cleaved by neutral endopeptidases (NEPs) (Knudsen and Pridel, 1996, Eur. J. Pharmacol. 318: 429-435 and Plamboeck et al., 2005, Diabetologia. 48: 1882-1890). Because a single parenteral injection of GLP-1 disappears from circulation within minutes, there is a need for a long-lasting, degradation-resistant GLP-1.

Recently, Exendin-4, a potent GLP-1 receptor agonist that is an endogenous product in the salivary glands of the Gila monster, was approved for treating type-2 diabetic patients (Parks et al., 2001, Metabolism. 50: 583-589; Aziz and Anderson, 2002, J. Nutr. 132: 990-995; and Egan et al., 2002, J. Clin. Endocrinol. Metab. 87: 1282-1290). Like GLP-1, it is insulinotropic, inhibits food intake and gastric emptying, and is trophic to β-cells in rodents. Further, due to the presence of glycine at position 2 of its N-terminus it is not a substrate for DPPIV. The downside to the use of Exendin-4 is that it must be injected twice daily because its t_{1/2} is only 2-4 hours (Kolterman et al., 2003, J. Clin. Endocrinol. Metab. 88: 3082-3089 and Fineman et al., 2003, Diabetes Care. 26: 2370-2377).

Accordingly, a need remains for a long-lasting, degradation resistant GLP-1 molecule. The present invention fulfills this need by providing transferrin and GLP-1 fusion proteins which extend the in vivo circulatory half-life of the GLP-1 protein while maintaining or increasing bioactivity.

### SUMMARY OF THE INVENTION

As described in more detail below, the present invention includes a fusion protein comprising a GLP-1 peptide, a substantially non-helical polypeptide linker, and a modified transferrin-in (mTf) molecule. In one embodiment, the fusion protein exhibits reduced glycosylation as compared to a native transferrin protein.

The linker moieties of the invention link a GLP-1 moiety to a modified transferrin moiety. In one embodiment, the linker is selected from the group consisting of PEAPTD (SEQ ID NO.: 13), PEAPTDPEAPTD (SEQ ID NO.: 10), PEAPTD in combination with an IgG hinge linker (SEQ ID NOS.: 118-123 and 126-129), and PEAPTDPEAPTD in combination with an IgG hinge linker.

The GLP-1 moiety of the invention may be modified. In one embodiment, the GLP-1 moiety may be modified to inhibit protease cleavage. For instance. a GLP-1 (7-36) or (7-36) moiety may be modified by mutating A8 to S, G or V (corresponds to A2 of SEQ ID NO.: 6) and /or mutating K34 to Q, A or N (corresponds to K28 of SEQ ID NO.: 6).

The GLP-1 /substantially non-helical polypeptide linker/mTf fusion protein of the present invention exhibits increased productivity of expression as compared to a similar fusion protein without a substantially non-helical linker. Further, the GLP-1/substantially non-helical polypeptide linker/mTf fusion protein of the present invention exhibits increased productivity of expression as compared to a similar fusion protein with a flexible polypeptide linker.

In another embodiment, the GLP-1/substantially non-helical polypeptide linker/mTf fusion protein exhibits a substantial increase in at least one activity as a result of the linker compared to a similar GLP-1/mTf fusion protein without a linker or compared to a similar GLP-1/mTf fusion protein with a flexible linker. For instance, the fusion protein of the invention may substantially reduce glucose levels and may substantially induce insulin secretion compared to a similar GLP-1/mTf fusion protein without a linker or with a flexible linker.

The fusion protein of the present invention also exhibits extended half-life and prolonged biological activity compared to a GLP-1 molecule in an unfused state.

It is envisioned that the fusion protein of the present invention may contain further modifications. For instance, the N-terminus of the fusion protein may contain a secretion signal sequence prior to cleavage. The Tf moiety may be modified so that it does not bind iron. Further, the Tf moiety may be modified so that it does not exhibit N-linked glycosylation or O-linked glycosylation. For instance, the mTf moiety may contain a mutation within or adjacent to the N-linked glycosylation site comprising the sequence N-X-S/T. In one embodiment, the mTf moiety contains a mutation at N413 and/or N611 or an adjacent S/T residue.

A nucleic acid molecule encoding the fusion protein of the present invention may be cloned into a vector and expressed in a host cell. In one embodiment of the invention, a host cell is cultured to express the fusion protein and the resulting protein is isolated. For instance, the fusion protein of the present invention can be expressed in yeast and then isolated and purified. A fusion protein of the present invention can also be isolated from a transgenic animal created using the nucleic acid molecule encoding the fusion protein of the present invention.

The present invention includes pharmaceutical compositions of the GLP-1/substantially non-helical polypeptide linker/mTf fusion protein. The invention includes methods of treating a patient suffering from type 2 diabetes, type 1 diabetes, obesity, congestive heart failure, non-fatty liver disease and other appropriate diseases by administering a pharmaceutical composition comprising a GLP-1/substantially non-helical polypeptide linker/mTf fusion protein. Administration of the fusion protein is useful for reducing glucose levels, inducing β cell proliferation and β-cell mass increase, inducing insulin secretion, decreasing gastric emptying, and increasing satiety.

### BRIEF DESCRIPTION OF THE DRAWINGS

FIG. 1 shows an alignment of the N and C Domains of Human (Hu) transferrin (Tf) (SEQ ID NO: 3) with similarities and identities highlighted.

FIGS. 2A-2B show an alignment of transferrin sequences from different species. Light shading: Similarity; Dark shading: Identity (SEQ ID NOS: 84-90).

FIG. 3 shows the location of a number of Tf surface exposed insertion sites for therapeutic proteins, polypeptides or peptides.

FIG. 4 is a diagram shows the different variants of the GLP-1, GLP-2 linker, and mTf used to construct the various GLP-1/GLP-2/mTf fusion proteins. In variants 2-10, GLP-1(-37)(variant 1, SEQ ID NO: 71) has been modified by amino acid deletion, N-terminal amino acid addition, and/or amino acid substitution. Variant 2 (amino acids 1-30 of SEQ ID NO: 71). Variant 3(amino acids 1-30 of SEQ ID NO: 71). Variant 4 (SEQ ID NO: 72). Variants 5-8 (SEQ ID NO: 73). Variant 9 (amino acids 1- 27). Variant 10 (amino acids 1-23 of SEQ ID NO: 71). In variants b-h, GLP-2 (Peptide a, SEQ ID NO: 74) has been modified. Variant b (amino acids 2-24 of SEQ ID NO: 74). Variants c-f (SEQ ID NO: 75). Variant g (amino acids 1-29 of SEQ ID 74). Variant h (amino acids 9-34 of SEQ ID NO: 74). The Example shows the N-terminal portion of fusion protein H GLP- 1(7-36)K34Q-GLP-2(2-34)K30A-mTf (SEQ ID NO: 75). In this Example, the second residue of mTf has also been modified from a Pro to a Ser.

FIG. 5 shows the relative potency in cAMP assay using rat GLP-1 receptor cloned into CHO cells.

FIG. 6 shows comparison of GLP-1/mTf plasma levels in monkeys measured by an ELISA and by an in vitro bioassay. Each point is the average of triplicate measurements. The GLP-1/mTf fusion protein used was GLP-1 (A8G,K34A)-PEAPTDPEAPTD-mTf.

FIG. 7 is a restriction map of pREX0585 (SEQ ID NO.: 11) encoding GLP-1-Tf (SEQ ID NO.: 12).

FIG. 8a shows the percent of maximal response of cAMP for various concentrations of GLP-1-Tf (SEQ ID NO.: 12), GLP-1, and Exendin-4 in GLP-1R transfected CHO cells.

FIG. 8b shows the percent of maximal secretion of insulin for various concentrations of GLP-1 and GLP-1-Tf (SEQ ID NO.: 12) in RIN 1046.38 cells.

FIG. 8c shows the percent of maximal response of insulin at various concentrations of GLP-1 and GLP-1-Tf in isolated rat islets.

FIG. 8d shows plasma GLP-1-Tf (SEQ ID NO.: 12) over time in cynomolgus monkeys treated with GLP-1-Tf by subcutaneous and intravenous routes of administration.

FIGS. 9a and b show blood glucose over time in non-diabetic mice treated with hTf, 1 mg/kg GLP-1-Tf, or 10 mg/Ig GLP-1-Tf by intraperitoneal (FIG. 9a) and subcutaneous (FIG. 9b) routes of administration.

FIGS. 10a through d show blood glucose and plasma insulin secretion levels in db/db mice treated with hTf or GLP-1-Tf (S EQ ID NO.: 12).

FIGS. 11a through d show blood glucose (FIGS. 11a and c) and food intake (FIGS. 11b and d) in db/db and non-diabetic mice fed once daily for 5 days after treatment with hTf or GLP-1-Tf (SEQ ID NO.: 12). FIG. 11a shows blood glucose in normal mice treated with hTf or GLP-1 over time. FIG. 11c shows blood glucose in db/db mice treated with hTf or GLP-1 over time. FIG. 11b shows food intake in non-diabetic mice treated with hTf or GLP-1-Tf over time. FIG. 11d shows food intake in db/db mice treated with hTf, GLP-1-Tf or Exendin-4 over time.

FIGS. 12a through c show BrdU+ nuclei in pancreata of db/db mice treated with hTf or GLP-1-Tf (SEQ ID NO.: 12).

FIG. 13a is a Western blot showing fragments corresponding to Tf and GLP-1-Tf in plasma from rats treated with Tf or GLP-1-Tf (SEQ ID NO.: 12).

FIGS. 13b through c are images of cFos activation in neurons of the area postrema, nuclei of solitary tract and paraventricular nuclei of the hypothalamus of rats treated with hTf or GLP-1-Tf (SEQ ID NO.: 12) by intraperitoneal injection (FIG. 13b) and intracerebroventricular injection (FIG. 13c).

FIG. 14 shows a 24 hour post treatment image of GLP-1-Tf in islets from fixed pancreata of mice treated with GLP-1-Tf or Tf using immunofluorescence and confocal microscopy.

### DETAILED DESCRIPTION

General Description

The present invention is based in part on the finding by the inventors that GLP-1 therapeutic proteins can be stabilized to extend their serum half-life and/or activity in vivo by genetically fusing the GLP-1 therapeutic proteins to transferrin, modified transferrin, or a portion of transferrin or modified transferrin sufficient to extend the half-life of the therapeutic protein in serum. The modified transferrin fusion proteins include a transferrin protein or domain covalently linked to a therapeutic protein or peptide, wherein the transferrin portion is modified to contain one or more amino acid substitutions, insertions or deletions compared to a wild-type transferrin sequence. In one embodiment, Tf fusion proteins are engineered to reduce or prevent glycosylation within the Tf or a Tf domain. In other embodiments, the Tf protein or Tf domain(s) is modified to exhibit reduced or no binding to iron or carbonate ion, or to have a reduced affinity or not bind to a Tf receptor (TfR).

The present invention is also based on the finding that inserting a linker between GLP1 and a transferrin molecule increases the stability and availability of the GLP-1 molecule for binding to its receptor. Specifically, it was found that using GLP-2 or a derivative thereof as a linker between GLP-1 and mTf improves the presentation of GLP-1 to its receptor. It was also found that using substantially non-helical linkers, including but not limited to PEAPTD, (PEAPTD)₂, PEAPTD in combination with an IgG hinge linker (SEQ ID NOS.: 118-123 and 126-129), and (PEAPTD)₂ in combination with an IgG hinge linker, substantially increases serum half-life, productivity of expression of the fusion protein and/or the activity of GLP-1. In one embodiment of the invention, a second GLP-1 or a derivative thereof may be used as a linker between a GLP-1 therapeutic protein and mTf.

The present invention therefore includes transferrin fusion proteins, therapeutic compositions comprising the fusion proteins, and methods of treating, preventing, or ameliorating diseases or disorders by administering the GLP-1 and transferrin fusion proteins. A GLP-1 and transferrin fusion protein of the invention includes at least a fragment or variant of a GLP-1 therapeutic protein, at least a fragment or variant of modified transferrin, and a linker which are associated with one another, preferably by genetic fusion (i.e., the transferrin fusion protein is generated by translation of a nucleic acid in which a polynucleotide encoding all or a portion of a therapeutic protein is joined in-frame with a polynucleotide encoding all or a portion of modified transferrin) or chemical conjugation to one another. The GLP-1 therapeutic protein, once part of the transferrin fusion protein, may be referred to as a GLP-1 “portion”, “region” or “moiety” of the transferrin fusion protein (e.g., a “GLP-1 therapeutic protein portion’ or a “transferrin protein portion”). Likewise, the substantially non-helical linker or GLP-2 linker, once part of the transferrin fusion protein, may be referred to as a “linker” or linker “portion”, “region” or “moiety” of the transferrin fusion protein.

In one embodiment, the invention provides a transferrin fusion protein comprising, or alternatively consisting of, a GLP-1 therapeutic protein, a linker protein, and a modified serum transferrin protein such as the fusion protein corresponding to SEQ ID NO.: 12. In other embodiments, the invention provides a transferrin fusion protein comprising, or alternatively consisting of, a biologically active and/or therapeutically active fragment of a GLP-1 therapeutic protein, a linker protein, and a modified transferrin protein. In other embodiments, the invention provides a transferrin fusion protein comprising, or alternatively consisting of, a biologically active and/or therapeutically active variant of a GLP-1 therapeutic protein, a linker protein, and modified transferrin protein. In further embodiments, the invention provides a transferrin fusion protein comprising a GLP-1 protein, a linker proteins and a biologically active and/or therapeutically active fragment of modified transferrin. In another embodiment, the GLP-1 therapeutic protein portion of the transferrin fusion protein is the active form of the therapeutic protein.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Definitions

As used herein, an “amino acid corresponding to” or an “equivalent amino acid” in a transferrin sequence is identified by alignment to maximize the identity or similarity between a first transferrin sequence and at least a second transferrin sequence. The number used to identify an equivalent amino acid in a second transferrin sequence is based on the number used to identify the corresponding amino acid in the first transferrin sequence. In certain cases, these phrases may be used to describe the amino acid residues in human transferrin compared to certain residues in rabbit serum transferrin.

As used herein, the term “biological activity” or “activity” refers to a function or set of activities performed by a GLP-1 therapeutic molecule, protein or peptide in a biological context (i.e., in an organism or an in vitro facsimile thereof). Biological activities may include but are not limited to the functions of the GLP-1 therapeutic molecule portion of the claimed fusion proteins, such as, but not limited to, the induction of insulin secretion, the lowering of blood glucose, the inhibition of food intake, the inhibition of gastric emptying, the increase in β-cell proliferation and β-cell mass, and the activation of cFos in the nervous system. A fusion protein or peptide of the invention is considered to be biologically active if it exhibits one or more biological activities of its therapeutic protein's native counterpart, i.e., unfused counterpart.

A fusion protein, with or without a GLP-2 and/or substantially non-helical linker, substantially exhibits prolonged biological activity if it exhibits one or more biological activities by at least about 10%, at least about 20%, at least about 30%, at least about 40%, at least about 50%, at least about 60%, at least about 70%, at least about 80%, at least about 90%, at least about 100%, at least about 200%, or at least about 300% or more, longer in duration, i.e., period of time, than the same one or more biological activities of its therapeutic protein's native counterpart, either in vivo or in vitro. In another embodiment, a fusion protein with a GLP-2 linker and/or substantially non-helical linker substantially exhibits prolonged activity if it exhibits one or more biological activities by at least about 10%, at least about 20%, at least about 30%, at least about 40%, at least about 50%, at least about 60%, at least about 70%, at least about 80%, at least about 90%, at least about 100%, at least about 200%, or at least about 300% or more, longer in duration, i.e., period of time, than the same one or more biological activities of a GLP-1 and Tf fusion protein lacking a linker sequence or a GLP-1 and Tf fusion protein with a flexible linker, either in vivo or in vitro.

As used herein, “binders” are agents used to impart cohesive qualities to the powdered material. Binders, or “granulators” as they are sometimes known, impart cohesiveness to the tablet formulation, which insures the tablet remaining intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and the like.

As used herein, the term “carrier” refers to a diluent, adjuvant, excipient, or vehicle with which a composition is administered. Such pharmaceutical carriers can be sterile liquids, such as water and oils, including those of petroleum, animal, vegetable or synthetic origin, such as peanut oil, soybean oil, mineral oil, sesame oil and the like.

As used herein, “coloring agents” are agents that give tablets a more pleasing appearance, and in addition help the manufacturer to control the product during its preparation and help the user to identify the product. Any of the approved certified water-soluble FD&C dyes, mixtures thereof, or their corresponding lakes may be used to color tablets. A color lake is the combination by adsorption of a water-soluble dye to a hydrous oxide of a heavy metal, resulting in an insoluble form of the dye.

As used herein, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and the like.

As used herein, “disintegrators” or “disintegrants” are substances that facilitate the breakup or disintegration of tablets after administration. Materials serving as disintegrants have been chemically classified as starches, clays, celluloses, algins, or gums. Other disintegrators include Veegum HV, methylcellulose, agar, bentonite, cellulose and wood products, natural sponge, cation-exchange resins, alginic acid, guar gum, citrus pulp, cross-linked polyvinylpyrrolidone, carboxymethylcellulose, and the like.

The term “dispersibility” or “dispersible” means a dry powder having a moisture content of less than about 10% by weight (% w) water, usually below about 5% w and preferably less than about 3% w; a particle size of about 1.0-5.0 μm mass median diameter (MMD), usually 1.0-4.0 μm MMD, and preferably 1.0-3.0 μm MMD; a delivered dose of about >30%, usually >40%, preferably >50%, and most preferred >60%; and an aerosol particle size distribution of 1.0-5.0 μm mass median aerodynamic diameter (MMAD), usually 1.5-4.5 μm MMAD, and preferably 1.5-4.0 μm MMAD.

The term “dry” means that the composition has a moisture content such that the particles are readily dispersible in an inhalation device to form an aerosol. This moisture content is generally below about 10% by weight (% w) water, usually below about 5% w and preferably less than about 3% w.

As used herein, “effective amount” means an amount of a drug or pharmacologically active agent that is sufficient to provide the desired local or systemic effect and performance at a reasonable benefit/risk ratio attending any medical treatment.

As used herein, “flavoring agents” vary considerably in their chemical structure, ranging from simple esters, alcohols, and aldehydes to carbohydrates and complex volatile oils. Synthetic flavors of almost any desired type are now available.

As used herein, the terms “fragment of a Tf protein” or “Tf protein,” or “portion of a Tf protein” refer to an amino acid sequence comprising at least about 5%, 10%, 20%, 30%, 40%, 50%, 60%, 70%, 80%, 90%, 95%, 96%, 97%, 98%, 99% or 100% of a naturally occurring Tf protein or mutant thereof.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, a “heterologous polynucleotide” or a “heterologous nucleic acid” or a “heterologous gene” or a “heterologous sequence” or an “exogenous DNA segment” refers to a polynucleotide, nucleic acid or DNA segment that originates from a source foreign to the particular host cell, or, if from the same source, is modified from its original form. A heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. As an example, a signal sequence native to a yeast cell but attached to a human Tf sequence is heterologous.

As used herein, an “isolated” nucleic acid sequence refers to a nucleic acid sequence which is essentially free of other nucleic acid sequences, e.g., at least about 20% pure, preferably at least about 40% pure, more preferably about 60% pure, even more preferably about 80% pure, most preferably about 90% pure, and even most preferably about 95% pure, as determined by agarose gel electrophoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated. The nucleic acid sequence may be of genomic, cDNA, RNA, semi-synthetic, synthetic origin, or any combinations thereof.

As used herein, two or more DNA coding sequences are said to be “joined” or “fused” when, as a result of in-frame fusions between the DNA coding sequences, the DNA coding sequences are translated into a fusion polypeptide. The term “fusion” in reference to Tf fusions includes, but is not limited to, attachment of at least one therapeutic protein, polypeptide or peptide to the N-terminal end of Tf, attachment to the C-terminal end of Tf, and/or insertion between any two amino acids within Tf.

As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils. Typical amounts of lubricants range from about 0.1 % by weight to about 5% by weight.

As used herein, “modified transferrin” as used herein refers to a transferrin molecule that exhibits at least one modification of its amino acid sequence, compared to wild-type transferrin.

As used herein, “modified transferrin fusion protein” as used herein refers to a protein formed by the fusion of at least one molecule of modified transferrin (or a fragment or variant thereof) to at least one molecule of a therapeutic protein (or fragment or variant thereof).

As used herein, the terms “nucleic acid” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene.

As used herein, a DNA segment is referred to as “operably linked” when it is placed into a functional relationship with another DNA segment. For example, DNA for a signal sequence is operably linked to DNA encoding a fusion protein of the invention if it is expressed as a preprotein that participates in the secretion of the fusion protein; a promoter or enhancer is operably linked to a coding sequence if it stimulates the transcription of the sequence. Generally, DNA sequences that are operably linked are contiguous, and in the case of a signal sequence or fusion protein both contiguous and in reading phase. However, enhancers need not be contiguous with the coding sequences whose transcription they control. Linking, in this context, is accomplished by ligation at convenient restriction sites or at adapters or linkers inserted in lieu thereof.

As used herein, “pharmaceutically acceptable” refers to materials and compositions that are physiologically tolerable and do not typically produce an allergic or similar untoward reaction, such as gastric upset, dizziness and the like, when administered to a human. Typically, as used herein, the tenn “pharmaceutically acceptable” means approved by a regulatory agency of the Federal or a state government or listed in the U.S. Pharmacopeia or other generally recognized pharmacopeia for use in animals, and more particularly in humans.

As used herein, “physiologically effective amount” is that amount delivered to a subject to give the desired palliative or curative effect. This amount is specific for each drug and its ultimate approved dosage level.

As used herein, “potency” refers to the ability of GLP-1 to activate one or more receptors. For instance, a GLP-1/substantially non-helical linker/mTF fusion protein of the invention can exhibit a potency at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least about 20 fold, or at least about 50 fold or more compared to a fusion protein without a substantially non-helical linker or compared to a fusion protein with a flexible linker.

As used herein, the “term powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 microns (μm) in diameter with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm.

As used herein, “productivity” of expression of the fusion protein refers to the ability of the protein to be expressed in a host cell system. For instance. the GLP-1 and Tf fusion protein with a GLP-2 linker or substantially non-helical linker can be expressed in a yeast cell. As used herein, “substantially increase productivity” means that expression of the fusion protein in the host is increased at least about 10%, at least about 20%, at least about 30%, at least about 40%, at least about 50%, at least about 60%, at least about 70%, at least about 80%, at least about 90%, at least about 100%, at least about 200%, or at least about 300% or more compared to the expression of a similar fusion protein lacking the linker or a similar fusion protein with a flexible polypeptide linker.

As used herein, the term “promoter” refers to a region of DNA involved in binding RNA polymerase to initiate transcription.

As used herein, “protease cleavage” refers to cleavage of GLP-1 by a protease. For instance, DPP-IV is a protease that naturally cleaves GLP-1. GLP-1(7-37) amino acid substitution A8G can prevent cleavage of the GLP-1 protein by DPP-IV at these amino acids. As used herein, a GLP-1 protein that has been modified to “substantially reduce protease cleavage” is a GLP-1 protein with one or more amino acid substitutions that exhibit at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, or at least about 15 fold increase or more reduction in protease cleavage compared to a native GLP-1 protein.

As used herein, the term “recombinant” refers to a cell, tissue or organism that has undergone transformation with a new combination of genes or DNA.

As used herein, the term “serum half-life” or “plasma half-life” refers to the time required for the in vivo serum GLP-1 concentration to decline by 50%. The shorter the serum half-life of GLP-1, the shorter win be the period that the protein can exert a therapeutic effect. For instance, a GLP-1 and Tf fusion peptide, with or without a GLP-2 linker and/or substantially non-helical linker, exhibits extended serum half-life if it exhibits a measurable increase in half-life including, at least about 5 fold, at least about 10 fold, at least about 50 fold, at least about 100 fold, at least about 200 fold, at least about 300 fold, at least about 400 fold, at least about 500 fold, at least about 600 fold, at least about 700 fold, at least about 800 fold, at least about 900 fold, at least about, 1000 fold, at least about 5,000 fold, at least about 10,000, at least about 25,000 fold, at least about 50,000 fold, at least about 75,000 fold, or at least about 100,000 fold increase or more in serum half-life compared to an unfused GLP-1 molecule.

As used herein, the term “subject” can be a human, a mammal, or an animal. The subject being treated is a patient in need of treatment.

As used herein, the term “substantially non-helical linker” or “rigid linker” refers to a linker that physically separates the GLP-1 and transferring moieties of a fusion protein. “Substantially non helical” means that linker peptide exhibits little or no helical or spiral shape or secondary structure. For instance, a substantially non-helical structure can comprise less than about 20% helical or spiral shape or secondary structure. A typical alpha-helical peptide is right-handed (twists in a clockwise direction), comprises the amino acid R groups extending to the outside of the helix, the helix making a complete turn at every 3.6 amino acids and the carbonyl group of each peptide bond extends parallel to the axis of the helix and points directly at the N−H group of the peptide bond 4 amino acids below it in the helix with a hydrogen bond forming between them. The non-helical linkers typically contain at least about 5%, at least about 10%, at least about 20%, at least about 30%, at least about 40%, at least about 50%, at least about 60%, at least about 70%, at least about 80%, at least about 90%, at least about 95%, or about 100% amino acids which disrupt any alpha-helix formation, such as amino acids that induce kinks in the polypeptide chain.

Kinks can be introduced into a naturally occurring peptide by modifying amino acid residues. Amino acids which cause kinks in the polypeptide chain include, for instance, proline and glycine amino acid residues. For example, the addition of a proline or a glycine at or about the middle of α straight c helical barrel win cause the protein to bend, i.e., kink. The introduction of a proline residue win generally cause a greater kink than the introduction of a glycine residue. As can be appreciated by a skilled artisan, the introduction of a proline or glycine residue anywhere in a linker peptide can cause a kink. A linker peptide can contain one or more amino acid residues which induce kinks. For instance, a substantially non-helical linker can have at least about 5% proline content, at least about 10% proline content, at least about 20% proline content, at least about 30% proline content, at least about 40% proline content, at least about 50% proline content, at least about 60% proline content, at least about 70% proline content, at least about 80% proline content, at least about 90% proline content, at least about 95% proline content or about 100% proline content.

Substantially non-helical linkers include, but are not limited to, PEAPTD (SEQ ID NO.: 13), PEAPTDPEAPTD (SEQ ID NO.: 10), PEAPTDPEAPTDPEAPTD (SEQ ID NO.: 14), IgG hinge (SEQ ID NO.: 88, 89, and 117), PEAPTD+IgG hinge (SEQ ID NOS.: 118-123 and 126-129), PPPPPPPPPPPP (SEQ ID NO.: 17), GEAPTDPEAPTD (SEQ ID NO.: 18), PEAGTDPEAPTD (SEQ ID NO.: 19), PEAPTDGEAPTD (SEQ ID NO.: 20), PEAPTDPEAGTD (SEQ ID NO.: 21), PQAPTNPQAPTN (SEQ ID NO.: 22), and PEAPEAPEAPEA (SEQ ID NO.: 23). Typically, substantially non-helical linkers have at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, or at least about 21 or more amino acids. However, there is no upper limit on linker length.

As used herein, “tablets” are solid pharmaceutical dosage forms containing drug substances with or without suitable diluents and prepared either by compression or molding methods well known in the art. Tablets have been in widespread use since the latter part of the 19^{th} century and their popularity continues. Tablets remain popular as a dosage form because of the advantages afforded both to the manufacturer (e.g., simplicity and economy of preparation, stability, and convenience in packaging, shipping, and dispensing) and the patient (e.g., accuracy of dosage, compactness, portability, blandness of taste, and ease of administration). Although tablets are most frequently discoid in shape, they may also be round, oval, oblong, cylindrical, or triangular. They may differ greatly in size and weight depending on the amount of drug substance present and the intended method of administration. They are divided into two general classes, (1) compressed tablets, and (2) molded tablets or tablet triturates. In addition to the active or therapeutic ingredient or ingredients, tablets contain a number or inert materials or additives. A first group of such additives includes those materials that help to impart satisfactory compression characteristics to the formulation, including diluents, binders, and lubricants. A second group of such additives helps to give additional desirable physical characteristics to the finished tablet, such as disintegrators, colors, flavors, and sweetening agents.

As used herein, the term “therapeutically effective amount” refers to that amount of the transferrin fusion protein comprising a GLP-1 therapeutic molecule which, when administered to a subject in need thereof, is sufficient to effect treatment. The amount of transferrin fusion protein which constitutes a “therapeutically effective amount” will vary depending on the therapeutic protein used, the severity of the condition or disease, and the age and body weight of the subject to be treated, but can be determined routinely by one of ordinary skin in the art having regard to his/her own knowledge and to this disclosure.

As used herein, “therapeutic protein” or “therapeutic molecule” refers to GLP-1, GLP-1 fragments or variants or analogs thereof, having one or more therapeutic and/or biological activities. The terms peptides, proteins, and polypeptides are used interchangeably herein. As used herein, a polypeptide displaying a “therapeutic activity” or a protein that is “therapeutically active” is GLP-1, GLP-1 fragment or a variant or analog thereof that possesses one or more known biological and/or therapeutic activities associated with GLP-1 such as described herein or otherwise known in the art. A “therapeutic protein” is a GLP-1 protein or analog that is useful to treat, prevent or ameliorate a disease, condition or disorder. Such a disease, condition or disorder may be in humans or in a non-human animal, e.g., veterinary use.

As used herein, the term “transformation” refers to the transfer of nucleic acid (i.e., a nucleotide polymer) into a cell. As used herein the term “genetic transformation” refers to the transfer and incorporation of DNA, especially recombinant DNA, into a cell.

As used herein, the term “transformant” refers to a cell, tissue or organism that has undergone transformation.

As used herein, the term “transgene” refers to a nucleic acid that is inserted into an organism, host cell or vector in a manner that ensures its function.

As used herein, the term “transgenic” refers to cells, cell cultures, organisms, bacteria, fungi, animals, plants, and progeny of any of the preceding, which have received a foreign or modified gene and in particular a gene encoding a modified Tf fusion protein by one of the various methods of transformation, wherein the foreign or modified gene is from the same or different species than the species of the organism receiving the foreign or modified gene.

“Variants or variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide. As used herein, “GLP-1 variant” or “GLP-1 analog” refers to a GLP-1 peptide, i.e., moiety, of a transferrin fusion protein of the invention, differing in sequence from a native therapeutic protein but retaining at least one functional and/or therapeutic property thereof as described elsewhere herein or otherwise known in the art. GLP-1 variants and analogs include a GLP- 1(7-37) containing one or more modifications to inhibit protease cleavage, including but not limited to the amino acid modifications A8G and K34A.

As used herein, the term “vector” refers broadly to any plasmid, phagemid or virus encoding an exogenous nucleic acid. The tenn is also be construed to include non-plasmid, non-phagemid and non-viral compounds which facilitate the transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; International Patent Application No. WO 94/17810, published Aug. 18, 1994; International Patent Application No. WO 94/23744, published Oct. 27, 1994). Examples of non-viral vectors include, but are not limited to, liposomes, polyamine derivatives of DNA, and the like.

As used herein, the term “wild type” refers to a polynucleotide or polypeptide sequence that is naturally occurring.

Transferrin and Transferrin Modifications

The present invention provides fusion proteins comprising GLP-1 or a GLP-1 analog and transferrin or modified transferrin. Any transferrin may be used to make modified Tf fusion proteins of the invention. In one embodiment, the Tf fusion protein also includes a GLP-2 and/or substantially non-helical linker. In another embodiment, the Tf fusion protein includes a GLP-1 linker in addition to the GLP-1 or GLP-1 analog therapeutic molecule.

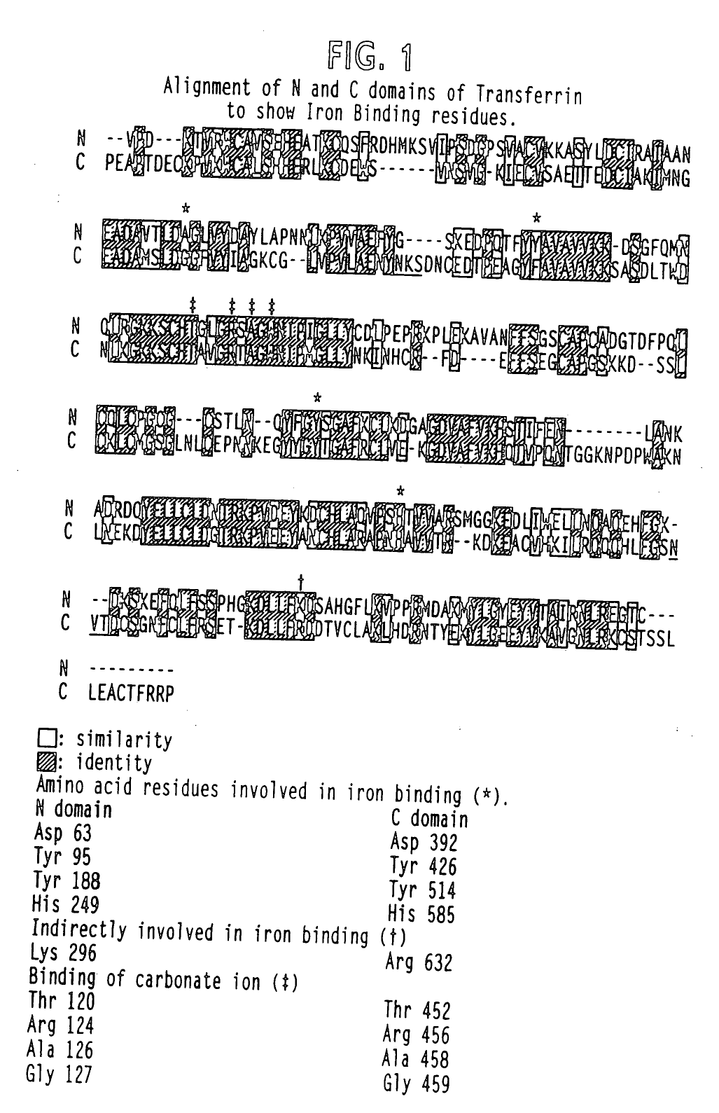

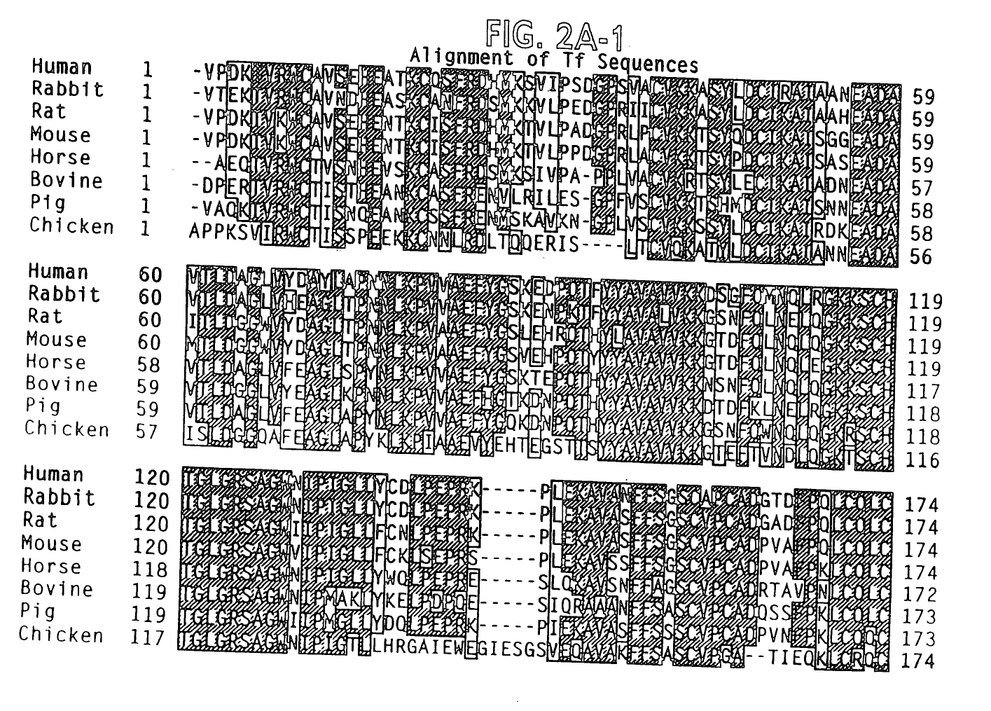

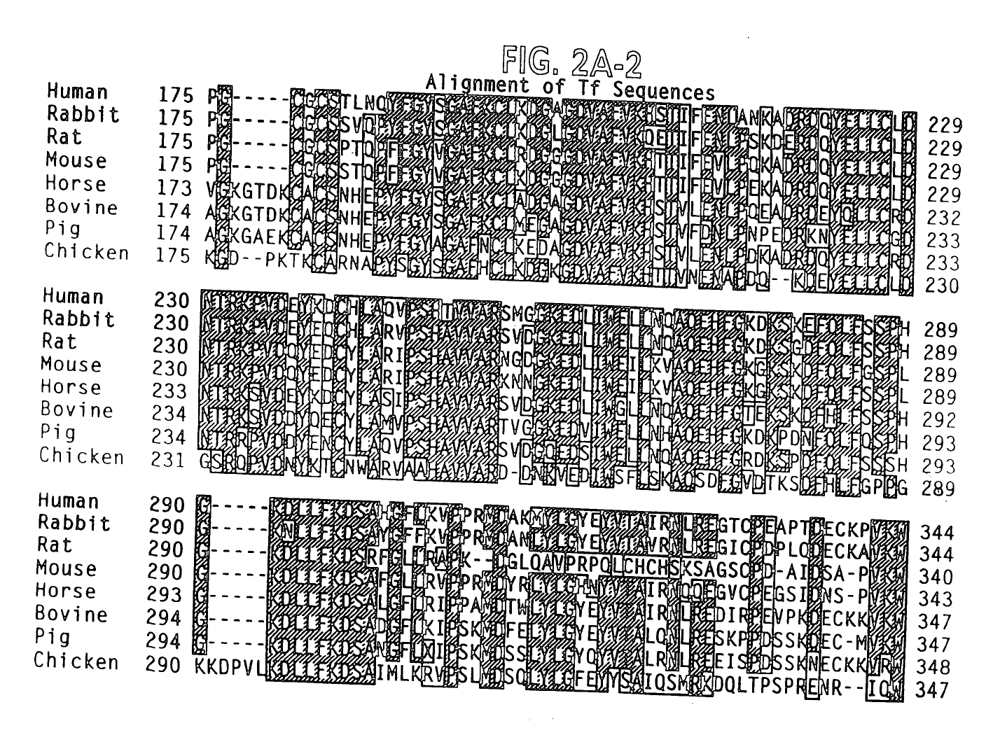

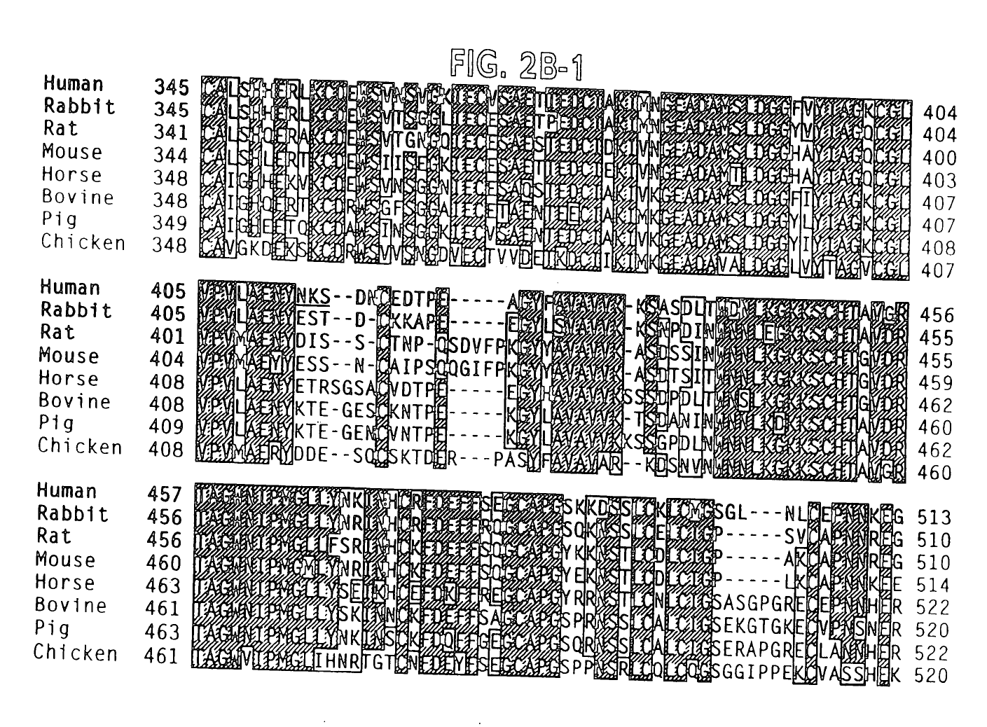

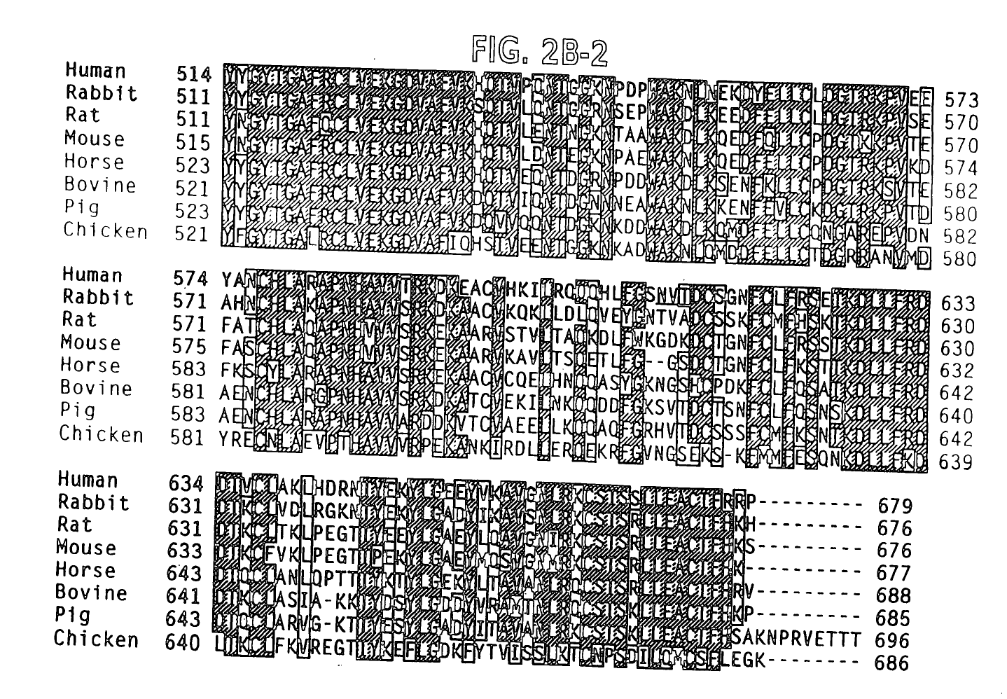

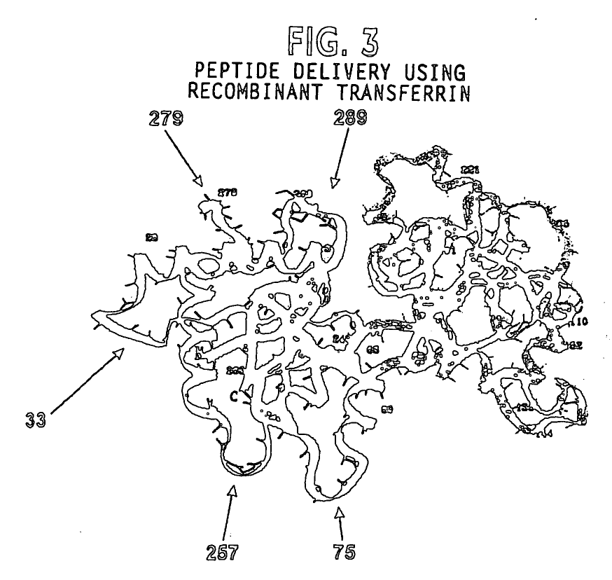

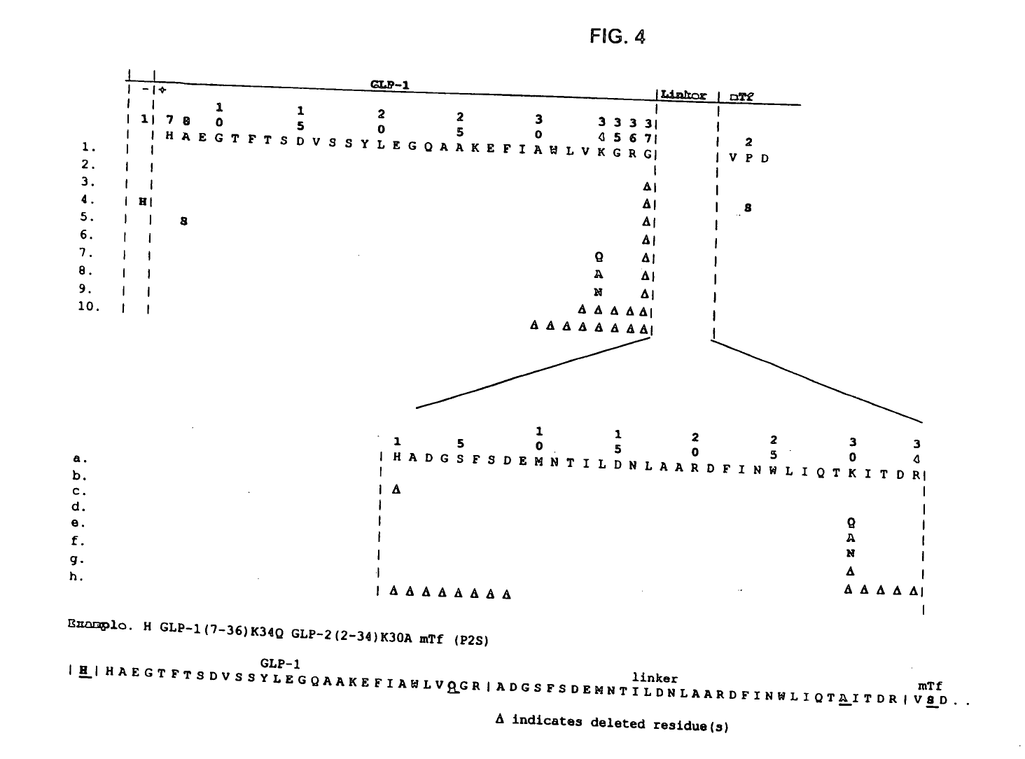

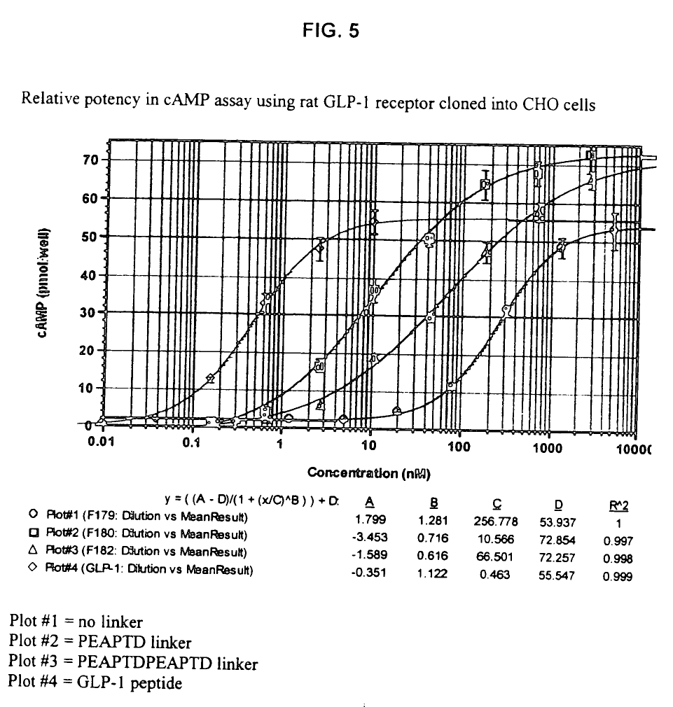

Any transferrin may be used to make modified Tf fusion proteins of the invention. As an example, the wild-type human Tf (Tf) is a 679 amino acid protein of approximately 75 kDa (not accounting for glycosylation), with two main domains, N (about 330 amino acids) and C (about 340 amino acids), which appear to originate from a gene duplication. See GenBank accession numbers NM_{—}001063, XM_{—}002793, M12530, XM_{—}039845, XM_{—}039847 and S95936 (www.ncbi.nlm.nih.gov/), all of which are herein incorporated by reference in their entirety, as well as SEQ ID NOS 1, 2 and 3. The two domains have diverged over time but retain a large degree of identity/similarity (FIG. 1).

Each of the N and C domains is further divided into two subdomains, N1 and N2, C1 and C2. The function of Tf is to transport iron to the cells of the body. This process is mediated by the Tf receptor (TfR), which is expressed on all cells, particularly actively growing cells. TfR recognizes the iron bound form of Tf (two molecules of which are bound per receptor), endocytosis then occurs whereby the TfR/Tf complex is transported to the endosome, at which point the localized drop in pH results in release of bound iron and the recycling of the TfR/Tf complex to the cell surface and release of Tf (known as apoTf in its iron-unbound form). Receptor binding is through the C domain of Tf. The two glycosylation sites in the C domain do not appear to be involved in receptor binding as unglycosylated iron bound Tf does bind the receptor.

Each Tf molecule can carry two iron ions (Fe³⁺). These are complexed in the space between the N1 and N2, C1 and C2 sub domains resulting in a conformational change in the molecule. Tf crosses the blood brain barrier (BBB) via the Tf receptor.

In human transferrin, the iron binding sites comprise at least amino acids Asp 63 (Asp 82 of SEQ ID NO: 2 which includes the native Tf signal sequence), Asp 392 (Asp 411 of SEQ ID NO: 2), Tyr 95 (Tyr 114 of SEQ ID NO: 2), Tyr 426 (Tyr 445 of SEQ ID NO: 2), Tyr 188 (Tyr 207 of SEQ ID NO: 2), Tyr 514 or 517 (Tyr 533 or Tyr 536 SEQ ID NO: 2), His 249 (His 268 of SEQ ID NO: 2), and His 585 (His 604 of SEQ ID NO: 2) of SEQ ID NO: 3. The hinge regions comprise at least N domain amino acid residues 94-96, 245-247 and/or 316-318 as well as C domain amino acid residues 425-427, 581-582 and/or 652-658 of SEQ ID NO: 3. The carbonate binding sites comprise at least amino acids Thr 120 (Thr 139 of SEQ ID NO: 2), Thr 452 (Thr 471 of SEQ ID NO: 2), Arg 124 (Arg 143 of SEQ ID NO: 2), Arg 456 (Arg 475 of SEQ ID NO: 2), Ala 126 (Ala 145 of SEQ ID NO: 2), Ala 458 (Ala 477 of SEQ ID NO: 2), Gly 127 (Gly 146 of SEQ ID NO: 2), and Gly 459 (Gly 478 of SEQ ID NO: 2) of SEQ ID NO: 3.

In one embodiment of the invention, the modified transferrin fusion protein includes a modified human transferrin, although any animal Tf molecule may be used to produce the fusion proteins of the invention, including human Tf variants, cow, pig, sheep, dog, rabbit, rat, mouse, hamster, echnida, platypus, chicken, frog, hornworm, monkey, as well as other bovine, canine and avian species. All of these Tf sequences are readily available in GenBank and other public databases. The human Tf nucleotide sequence is available (see SEQ ID NOS 1, 2 and 3 and the accession numbers described above and available at www.ncbi.nlm.nih.gov/) and can be used to make genetic fusions between Tf or a domain of Tf and the therapeutic molecule of choice. Fusions may also be made from related molecules such as lacto transferrin (lactoferrin) GenBank Acc: NM_{—}002343) or melanotransferrin (GenBank Acc. NM_{—}013900, murine melanotransferrin).

Melanotransferrin is a glycosylated protein found at high levels in malignant melanoma cells and was originally named human melanoma antigen p97 (Brown et al., 1982, Nature, 296: 171-173). It possesses high sequence homology with human serum transferrin, human lactoferrin, and chicken transferrin (Brown et al., 1982, Nature, 296: 171-173; Rose et al., Proc. Natl. Acad. Sci. USA, 1986, 83: 1261-1265). However, unlike these receptors, no cellular receptor has been identified for melanotransferrin. Melanotransferrin reversibly binds iron and it exists in two forms, one of which is bound to cell membranes by a glycosyl phosphatidylinositol anchor while the other form is both soluble and actively secreted (Baker et al., 1992, FEBS Lett, 298: 215-218; Alemany et al., 1993, J. Cell Sci., 104: 1155-1162; Food et al., 1994, J. Biol. Chem. 274: 7011-7017).

Lactoferrin (Lf), a natural defense iron-binding protein, has been found to possess antibacterial, antimycotic, antiviral, antineoplastic and anti-inflammatory activity. The protein is present in exocrine secretions that are commonly exposed to normal flora: milk, tears, nasal exudate, saliva, bronchial mucus, gastrointestinal fluids, cervico-vaginal mucus and seminal fluid. Additionally, Lf is a major constituent of the secondary specific granules of circulating polymorphonuclear neutroplils (PMNs). The apoprotein is released on degranulation of the PMNs in septic areas. A principal function of Lf is that of scavenging free iron in fluids and inflamed areas so as to suppress free radical-mediated damage and decrease the availability of the metal to invading microbial and neoplastic cells. In a study that examined the turnover rate of ¹²⁵I Lf in adults, it was shown that Lf is rapidly taken up by the liver and spleen, and the radioactivity persisted for several weeks in the liver and spleen (Bennett et al. (1979), Clin. Sci. (Lond.) 57: 453-460).

In one embodiment, the transferrin portion of the transferrin fusion protein of the invention includes a transferrin splice variant. In one example, a transferrin splice variant can be a splice variant of human transferrin. In one specific embodiment, the human transferrin splice variant can be that of Genbank Accession AAA61140.

In another embodiment, the transferrin portion of the transferrin fusion protein of the invention includes a lactoferrin splice variant. In one example, a human serum lactoferrin splice variant can be a novel splice variant of a neutrophil lactoferrin. In one specific embodiment, the neutrophil lactoferrin splice variant can be that of Genbank Accession AAA59479. In another specific embodiment, the neutrophil lactoferrin splice variant can comprise the following amino acid sequence EDCIALKGEADA (SEQ ID NO: 5), which includes the novel region of splice-variance.

In another embodiment, the transferrin portion of the transferrin fusion protein of the invention includes a melanotransferrin variant.

Modified Tf fusions may be made with any Tf protein, fragment, domain, or engineered domain. For instance, fusion proteins may be produced using the full-length Tf sequence, with or without the native Tf signal sequence. Tf fusion proteins may also be made using a single Tf domain, such as an individual N or C domain or a modified form of Tf comprising 2N or 2C domains (see U.S. Provisional Application 60/406,977, filed Aug. 30, 2002, which is herein incorporated by reference in its entirety). In some embodiments, fusions of a therapeutic protein to a single C domain may be produced, wherein the C domain is altered to reduce, inhibit or prevent glycosylation. In other embodiments, the use of a single N domain is advantageous as the Tf glycosylation sites reside in the C domain and the N domain, on its own. A preferred embodiment is the Tf fusion protein having a single N domain which is expressed at a high level.

As used herein, a C terminal domain or lobe modified to function as an N-like domain is modified to exhibit glycosylation patterns or iron binding properties substantially like that of a native or wild-type N domain or lobe. In a preferred embodiment, the C domain or lobe is modified so that it is not glycosylated and does not bind iron by substitution of the relevant C domain regions or amino acids to those present in the corresponding regions or sites of a native or wild-type N domain.

As used herein, a Tf moiety comprising “two N domains or lobes” includes a Tf molecule that is modified to replace the native C domain or lobe with a native or wild-type domain or lobe or a modified N domain or lobe or contains a C domain that has been modified to function substantially like a wild-type or modified N domain.

Analysis of the two domains by overlay of the two domains (Swiss PDB Viewer 3.7b2, Iterative Magic Fit) and by direct amino acid alignment (ClustalW multiple alignment) reveals that the two domains have diverged over time. Amino acid alignment shows 42% identity and 59% similarity between the two domains. However, approximately 80% of the N domain matches the C domain for structural equivalence. The C domain also has several extra disulfide bonds compared to the N domain.

Alignment of molecular models for the N and C domain reveals the following structural equivalents:

| | | | | | | | | | | | | | | |
|---|---|---|---|---|---|---|---|---|---|---|---|---|---|---|
| N | 4- | 36- | 94- | 138- | 149- | 168- | 178- | 219- | 259- | 263- | 271- | 279- | 283- | 309- |
| domain | 24 | 72 | 136 | 139 | 164 | 173 | 198 | 255 | 260 | 268 | 275 | 280 | 288 | 327 |
| (1-330) | | 75- | | | | | 200- | | | | | | 290- | |
| | | 88 | | | | | 214 | | | | | | 304 | |
| C | 340- | 365- | 425- | 470- | 475- | 492- | 507- | 555- | 593- | 597- | 605- | 614- | 620- | 645- |
| domain | 361 | 415 | 437 | 471 | 490 | 497 | 542 | 591 | 594 | 602 | 609 | 615 | 640 | 663 |
| (340- | | | 439- | | | | | | | | | | | |
| 679) | | | 468 | | | | | | | | | | | |

The disulfide bonds for the two domains align as follows:

| N | C |
|---|---|
| | C339-C596 |
| C9-C48 | C345-C377 |
| C19-C39 | C355-C368 |
| | C402-C674 |
| | C418-C637 |
| C118-C194 | C450-C523 |
| C137-C331 | |
| | C474-C665 |
| C158-C174 | C484-C498 |
| C161-C179 | |
| C171-C177 | C495-C506 |
| C227-C241 | C563-C577 |
| | C615-C620 |

Bold aligned disulfide bonds
Italics bridging peptide

In one embodiment, the transferrin portion of the transferrin fusion protein includes at least two N terminal lobes of transferrin. In further embodiments, the transferrin portion of the transferrin fusion protein includes at least two N terminal lobes of transferrin derived from human serum transferrin.

In another embodiment, the transferrin portion of the transferrin fusion protein includes, comprises, or consists of at least two N terminal lobes of transferrin having a mutation in at least one amino acid residue selected from the group consisting of Asp63, Gly65, Tyr95. Tyrl 88, and His249 of SEQ ID NO: 3.

In another embodiment, the transferrin portion of the modified transferrin fusion protein includes a recombinant human serum transferrin N-terminal lobe mutant having a mutation at Lys206 or His207 of SEQ ID NO: 3.

In another embodiment, the transferrin portion of the transferrin fusion protein includes, comprises, or consists of at least two C terminal lobes of transferrin. In further embodiments, the transferrin portion of the transferrin fusion protein includes at least two C terminal lobes of transferrin derived from human serum transferrin.

In a further embodiment, the C terminal lobe mutant further includes a mutation of at least one of Asn413 and Asn611 of SEQ ID NO: 3 which does not allow glycosylation.

In another embodiment, the transferrin portion of the transferrin fusion protein includes at least two C terminal lobes of transferrin having a mutation in at least one amino acid residue selected from the group consisting of Asp392, Tyr426, Tyr514, Tyr517 and His585 of SEQ ID NO: 3, wherein the mutant retains the ability to bind metal. In an alternate embodiment, the transferrin portion of the transferrin fusion protein includes at least two C terminal lobes of transferrin having a mutation in at least one amino acid residue selected from the group consisting of Tyr426, Tyr514, Tyr517 and His585 of SEQ ID NO: 3, wherein the mutant has a reduced ability to bind metal. In another embodiment, the transferrin portion of the transferrin fusion protein includes at least two C terminal lobes of transferrin having a mutation in at least one amino acid residue selected from the group consisting of Asp392, Tyr426, Tyr517 and His585 of SEQ ID NO:3, wherein the mutant does not retain the ability to bind metal and functions substantially like an N domain.

In some embodiments, the Tf or Tf portion win be of sufficient length to increase the in vivo circulatory half-life, serum stability, in vitro solution stability or bioavailability of the GLP-1 therapeutic protein compared to the in vivo circulatory half-life, serum stability, in vitro solution stability or bioavailability of the GLP-1 therapeutic protein in an unfused state. Such an increase in stability, serum half-life or bioavailability may be about 5 fold, 10 fold, 50 fold, 100 fold, 200 fold, 300 fold, 400 fold, 500 fold, 600 fold, 700 fold, 800 fold, 900 fold, 1,000 fold, 5,000 fold, 10,000, 25,000 fold, 50,000 fold, 75,000 fold, or 100,000 fold or more. In some cases, the transferrin fusion proteins comprising modified transferrin and GLP-1 and optionally a GLP-2 and/or substantially rigid linker exhibit a serum half-life of about 12-24 hours, about 18-24 hours, about 1 day, about 30 hours, about 38 hours, about 40 hours, about 42 hours, about 45 hours, about 2 days, about 3 days, about 4 days, about 5 days, about 6 days, about 7 days, about 8 days, about 9 days, about 10-20 or more days, about 12-18 days or about 14-17 days.

When the C domain of Tf is part of the fusion protein, the two N-linked glycosylation sites, amino acid residues corresponding to N413 and N611 of SEQ ID NO: 3 may be mutated for expression in a yeast system to prevent glycosylation or hypermannosylationn and extend the serum half-life of the fusion protein and/or therapeutic protein (to produce asialo-, or in some instances, monosialo-Tf or disialo-Tf). In addition to Tf amino acids corresponding to N413 and N611, mutations may be to the adjacent residues within the N-X-S/T glycosylation site to prevent or substantially reduce glycosylation. See U.S. Pat. No. 5,986,067 of Funk et al. It has also been reported that the N domain of Tf expressed in Pichia pastoris becomes O-linked glycosylated with a single hexose at S32 which also may be mutated or modified to prevent such glycosylation.

Accordingly, in one embodiment of the invention, the transferrin fusion protein includes a modified transferrin molecule wherein the transferrin exhibits reduced glycosylation, including but not limited to asialo- monosialo- and disialo- forms of Tf. In another embodiment, the transferrin portion of the transferrin fusion protein includes a recombinant transferrin mutant that is mutated to prevent glycosylation. In another embodiment, the transferrin portion of the transferrin fusion protein includes a recombinant transferrin mutant that is fully glycosylated. In a further embodiment, the transferrin portion of the transferrin fusion protein includes a recombinant human serum transferrin mutant that is mutated to prevent N-linked glycosylation, wherein at least one of Asn413 and Asn611 of SEQ ID NO: 3 are mutated to an amino acid which does not allow glycosylation. In another embodiment, the transferrin portion of the transferrin fusion protein includes a recombinant human serum transferrin mutant that is mutated to prevent or substantially reduce glycosylation, wherein mutations may be to the adjacent residues within the N-X-S/T glycosylation site, for instance mutation of the S/T residues. Moreover, glycosylation may be reduced or prevented by mutating the serine or threonine residue. Further, changing the X to proline is known to inhibit glycosylation.

As discussed below in more detail, modified Tf fusion proteins of the invention may also be engineered to not bind iron and/or bind the Tf receptor. In other embodiments of the invention, the iron binding is retained and the iron binding ability of Tf may be used to deliver a therapeutic protein or peptide(s) to the inside of a cell, across an epithelial or endothelial cell membrane and/or across the BBB. These embodiments that bind iron and/or the Tf receptor win often be engineered to reduce or prevent glycosylation to extend the serum half-life of the therapeutic protein. The N domain alone win not bind to TfR when loaded with iron, and the iron bound C domain win bind TfR but not with the same affinity as the whole molecule.

In another embodiment, the transferrin portion of the transferrin fusion protein includes a recombinant transferrin mutant having a mutation wherein the mutant does not retain the ability to bind metal ions. In an alternate embodiments the transferrin portion of the transferrin fusion protein includes a recombinant transferrin mutant having a mutation wherein the mutant has a weaker binding affinity for metal ions than wild-type serum transferrin. In an alternate embodiment, the transferrin portion of the transferrin fusion protein includes a recombinant transferrin mutant having a mutation wherein the mutant has a stronger binding affinity for metal ions than wild-type serum transferrin.

In another embodiment, the transferrin portion of the transferrin fusion protein includes a recombinant transferrin mutant having a mutation wherein the mutant does not retain the ability to bind to the transferrin receptor. For instance, the modified GLP-1 and Tf fusion proteins of the invention may bind a cell surface GLP-1 R receptor but not a Tf receptor. Such fusion proteins can be therapeutically active at the cell surface, i.e., by not by entering the cell.

In an alternate embodiment, the transferrin portion of the transferrin fusion protein includes a recombinant transferrin mutant having a mutation wherein the mutant has a weaker binding affinity for the transferrin receptor than wild-type serum transferrin. In an alternate embodiment, the transferrin portion of the transferrin fusion protein includes a recombinant transferrin mutant having a mutation wherein the mutant has a stronger binding affinity for the transferrin receptor than wild-type serum transferrin.

In another embodiment, the transferrin portion of the transferrin fusion protein includes a recombinant transferrin mutant having a mutation wherein the mutant does not retain the ability to bind to carbonate ions. In an alternate embodiment, the transferrin portion of the transferrin fusion protein includes a recombinant transferrin mutant having a mutation wherein the mutant has a weaker binding affinity for carbonate ions than wild-type serum transferrin. In an alternate embodiment, the transferrin portion of the transferrin fusion protein includes a recombinant transferrin mutant having a mutation wherein the mutant has a stronger binding affinity for carbonate ions than wild-type serum transferrin.

In another embodiment, the transferrin portion of the transferrin fusion protein includes a recombinant human serum transferrin mutant having a mutation in at least one amino acid residue selected from the group consisting of Asp63, Gly65, Tyr95, Tyr188, His249, Asp392, Tyr426, Tyr514, Tyr517 and His585 of SEQ ID NO: 3, wherein the mutant retains the ability to bind metal ions. In an alternate embodiment, a recombinant human serum transferrin mutant having a mutation in at least one amino acid residue selected from the group consisting of Asp63, Gly65, Tyr95, Tyr188, His249, Asp392, Tyr426, Tyr514, Tyr517 and His585 of SEQ ID NO: 3, wherein the mutant has a reduced ability to bind metal ions. In another embodiment, a recombinant human serum transferrin mutant having a mutation in at least one amino acid residue selected from the group consisting of Asp63, Gly65, Tyr95, Tyr188, His249, Asp392, Tyr426, Tyr517 and His585 of SEQ ID NO: 3, wherein the mutant does not retain the ability to bind metal ions.

In another embodiment, the transferrin portion of the transferrin fusion protein includes a recombinant human serum transferrin mutant having a mutation at Lys206 or His207 of SEQ ID NO:3, wherein the mutant has a stronger binding affinity for metal ions than wild-type human serum transferrin (see U.S. Pat. No. 5,986,067, which is herein incorporated by reference in its entirety). In an alternate embodiment, the transferrin portion of the transferrin fusion protein includes a recombinant human serum transferrin mutant having a mutation at Lys206 or His207 of SEQ ID NO:3, wherein the mutant has a weaker binding affinity for metal ions than wild-type human serum transferrin. In a further embodiment, the transferrin portion of the transferrin fusion protein includes a recombinant human serum transferrin mutant having a mutation at Lys206 or His207 of SEQ ID NO:3, wherein the mutant does not bind metal ions.

Any available technique may be used to produce the transferrin fusion proteins of the invention, including but not limited to molecular techniques commonly available, for instance, those disclosed in Sambrook et al. Molecular Cloning: A Laboratory Manual, 2nd Ed., Cold Spring Harbor Laboratory Press, 1989. When carrying out nucleotide substitutions using techniques for accomplishing site-specific mutagenesis that are well known in the art, the encoded amino acid changes are preferably of a minor nature, that is, conservative amino acid substitutions, although other, non-conservative, substitutions are contemplated as well, particularly when producing a modified transferrin portion of a Tf fusion protein, e.g., a modified Tf protein exhibiting reduced glycosylation, reduced iron binding and the like. Specifically contemplated are amino acid substitutions, small deletions or insertions, typically of one to about 30 amino acids; insertions between transferrin domains; small amino- or carboxyl-terminal extensions, such as an amino-terminal methionine residue, or small linker peptides of less than 50, 40, 30, 20 or 10 residues between transferrin domains or linking a transferrin protein and therapeutic protein or peptide or a small extension that facilitates purification, such as a poly-histidine tract, an antigenic epitope or a binding domain.

Examples of conservative amino acid substitutions are substitutions made within the same group such as within the group of basic amino acids (such as arginine, lysine, histidine), acidic amino acids (such as glutamic acid and aspartic acid), polar amino acids (such as glutamine and asparagine), hydrophobic amino acids (such as leucine, isoleucine, valine), aromatic amino acids (such as phenylalanine, tryptophan, tyrosine) and small amino acids (such as glycine, alanine, serine, threonine, methionine).

Non-conservative substitutions encompass substitutions of amino acids in one group by amino acids in another group. For example, a non-conservative substitution would include the substitution of a polar amino acid for a hydrophobic amino acid. For a general description of nucleotide substitution, see e.g. Ford et al. (1991), Prot. Exp. Pur. 2: 95-107. Non-conservative substitutions, deletions and insertions are particularly useful to produce Tf fusion proteins of the invention that exhibit no or reduced binding of iron, no or reduced binding of the fusion protein to the Tf receptor and/or no or reduced glycosylation.

Iron binding and/or receptor binding may be reduced or disrupted by mutation, including deletion, substitution or insertion into, amino acid residues corresponding to one or more of Tf N domain residues Asp63, Tyr95, Tyr188, His249 and/or C domain residues Asp 392, Tyr 426, Tyr 514 and/or His 585 of SEQ ID NO: 3. Iron binding may also be affected by mutation to amino acids Lys206, His207 or Arg632 of SEQ ID NO: 3. Carbonate binding may be reduced or disrupted by mutation, including deletion, substitution or insertion into, amino acid residues corresponding to one or more of Tf N domain residues Thr120, Arg124, Ala126, Gly 127 and/or C domain residues Thr 452, Arg 456, Ala 458 and/or Gly 459 of SEQ ID NO: 3. A reduction or disruption of carbonate binding may adversely affect iron and/or receptor binding.

Binding to the Tf receptor may be reduced or disrupted by mutation, including deletion, substitution or insertion into, amino acid residues corresponding to one or more of Tf N domain residues described above for iron binding.

As discussed above, glycosylation may be reduced or prevented by mutation, including deletion, substitution or insertion into, amino acid residues corresponding to one or more of Tf C domain residues around the N-X-S/T sites corresponding to C domain residues N413 and/or N611 (See U.S. Pat. No. 5,986,067). For instance, the N413 and/or N611 may be mutated to Glu residues.

In instances where the Tf fusion proteins of the invention are not modified to prevent glycosylation, iron binding, carbonate binding and/or receptor binding, glycosylation, iron and/or carbonate ions may be stripped from or cleaved off of the fusion protein. For instance, available deglycosylases may be used to cleave glycosylation residues from the fusion protein, in particular the sugar residues attached to the Tf portion, yeast deficient in glycosylation enzymes may be used to prevent glycosylation and/or recombinant cells may be grown in the presence of an agent that prevents glycosylation, e.g., tunicamycin.

The carbohydrates on the fusion protein may also be reduced or completely removed enzymatically by treating the fusion protein with deglycosylases. Deglycosylases are well known in the art. Examples of deglycosylases include but are not limited to galactosidase, PNGase A, PNGase F, glucosidase, mannosidase, fucosidase, and Endo H deglycosylase.

Nevertheless, in certain circumstances, it may be preferable for oral delivery that the Tf portion of the fusion protein be fully glycosylated

Additional mutations may be made with Tf to alter the three dimensional structure of Tf, such as modifications to the hinge region to prevent the conformational change needed for iron biding and Tf receptor recognition. For instance, mutations may be made in or around N domain amino acid residues 94-96, 245-247 and/or 316-318 as well as C domain amino acid residues 425-427, 581-582 and/or 652-658. In addition, mutations may be made in or around the flanking regions of these sites to alter Tf structure and function.

In one aspect of the invention, the transferrin fusion protein can function as a carrier protein to extend the half life or bioavailability of the therapeutic protein as well as, in some instances, delivering the therapeutic protein inside a cell and/or across the blood brain barrier. In an alternate embodiment, the transferrin fusion protein includes a modified transferrin molecule wherein the transferrin does not retain the ability to cross the blood brain barrier.

In another embodiment, the transferrin fusion protein includes a modified transferrin molecule wherein the transferrin molecule retains the ability to bind to the transferrin receptor and transport the therapeutic peptide inside cells. In an alternate embodiment, the transferrin fusion protein includes a modified transferrin molecule wherein the transferrin molecule does not retain the ability to bind to the transferrin receptor and transport the therapeutic peptide inside cells.

In further embodiments, the transferrin fusion protein includes a modified transferrin molecule wherein the transferrin molecule retains the ability to bind to the transferrin receptor and transport the therapeutic peptide inside cells and retains the ability to cross the blood brain barrier. In an alternate embodiment, the transferrin fusion protein includes a modified transferrin molecule wherein the transferrin molecule retains the ability to cross the blood brain barrier, but does not retain the ability to bind to the transferrin receptor and transport the therapeutic peptide inside cells.

Modified Transferrin Fusion Proteins

The fusion of proteins of the invention may contain one or more copies of the GLP1 therapeutic protein such as GLP-1 or an analog thereof attached to the N-terminus and/or the C-terminus of the Tf protein. In some embodiments, the GLP1 therapeutic protein is attached to both the N- and C-terminus of the Tf protein and the fusion protein may contain one or more equivalents of the GLP-1 therapeutic protein or polypeptide on either or both ends of Tf. In other embodiments, the GLP-1 therapeutic protein or polypeptide is inserted into known domains of the Tf protein, for instance, into one or more of the loops of Tf (see All et al. (1999) J. Biol. Chem. 274(34):24066-24073). In fact, the GLP-1 therapeutic protein or polypeptide may be inserted into all five loops of transferrin to create a pentavalent molecule with increased affinity for the GLP-1 receptor. In other embodiments, the GLP-1 therapeutic protein or polypeptide is inserted between the N and C domains of Tf. Alternatively, the GLP-1 therapeutic protein or polypeptide is inserted anywhere in the transferrin molecule.

The fusion protein of the present invention includes the use of a GLP-2 linker and/or substantially non-helical amino acid linker to separate the transferrin and GLP-1 moieties of the protein. For instance, in one embodiment, GLP-1 is attached to the N-terminus of Tf with an intervening linker peptide. In another embodiment, GLP-1 is attached to the C-terminus of Tf with an intervening linker peptide. As can be appreciated by a skilled artisan, the invention envisions a linker being situated numerous ways in the molecule to separate GLP-1 and transferrin.

Generally, the transferrin fusion protein of the invention may lave one modified transferrin-derived region and one GLP-1 therapeutic protein region. Multiple regions of each protein, however, may be used to make a transferrin fusion protein of the invention. Similarly, more than one GLP-1 therapeutic protein may be used to make a transferrin fusion protein of the invention, thereby producing a multi-functional modified Tf fusion protein.

In one embodiment, the transferrin fusion protein of the invention contains a GLP-1 therapeutic protein fused to a transferrin molecule or portion thereof. In another embodiment, the transferrin fusion protein of the inventions contains a GLP-1 therapeutic protein or polypeptide fused to the N terminus of a transferrin molecule. In an alternate embodiment, the transferrin fusion protein of the invention contains a GLP-1 therapeutic protein or polypeptide fused to the C terminus of a transferrin molecule. In a further embodiment, the transferrin fusion protein of the invention contains a transferrin molecule fused to the N terminus of a GLP-1 therapeutic protein or polypeptide. In an alternate embodiment, the transferrin fusion protein of the invention contains a transferrin molecule fused to the C terminus of a GLP-1 therapeutic protein or polypeptide.

In other embodiments, the transferrin fusion protein of the inventions contains a GLP-1 therapeutic protein fused to both the N-terminus and the C-terminus of modified transferrin. In an alternate embodiment, the therapeutic proteins fused at the N- and C-termini include one or more GLP-1 therapeutic protein and one or more different therapeutic proteins which may be used to treat or prevent disease or disorders which are known in the art to be treatable with GLP-1 . In another embodiment, the therapeutic proteins fused at the N- and C-termini are one or more GLP-1 therapeutic proteins and one or more different therapeutic proteins which may be used to treat or prevent diseases or disorders which are known in the art to commonly occur in patients simultaneously.

In addition to modified transferrin fusion protein of the invention in which the modified transferrin portion is fused to the N terminal and/or C-terminal of the GLP-1 therapeutic protein portion, transferrin fusion protein of the invention may also be produced by inserting the GLP-1 therapeutic protein or peptide of interest (e.g., a therapeutic protein or peptide as disclosed herein, or a fragment or variant thereof) into an internal region of the modified transferrin. Internal regions of modified transferrin include, but are not limited to, the iron binding sites, the hinge regions, the bicarbonate binding sites, or the receptor binding domain.

Within the protein sequence of the modified transferrin molecule a number of loops or turns exist, which are stabilized by disulfide bonds. These loops are useful for the insertion, or internal fusion, of therapeutically active peptides particularly those requiring a secondary structure to be functional, or therapeutic proteins to generate a modified transferrin molecule with specific biological activity.

When therapeutic proteins are inserted into or replace at least one loop of a Tf molecule, insertions may be made within any of the surface exposed loop regions, in addition to other areas of Tf. For instance, insertions may be made within the loops comprising Tf amino acids 32-33, 74-75, 256-257, 279-280 and 288-289 (Ali et al., supra) (See FIG. 3). As previously described, insertions may also be made within other regions of Tf such as the sites for iron and bicarbonate binding, hinge regions, and the receptor binding domain as described in more detail below. The loops in the Tf protein sequence that are amenable to modification/replacement for the insertion of proteins or peptides may also be used for the development of a screenable library of random peptide inserts. Any procedures may be used to produce nucleic acid inserts for the generation of peptide libraries, including available phage and bacterial display systems, prior to cloning into a Tf domain and/or fusion to the ends of Tf.

The N-terminus of Tf is free and points away from the body of the molecule. Fusions of proteins or peptides on the N-terminus may therefore be a preferred embodiment. Such fusions may include a GLP-2 and/or substantially non-helical linker to separate the GLP-1 therapeutic protein from Tf.

The C-terminus of Tf appears to be more buried and secured by a disulfide bond 6 amino acids from the C-terminus. In human Tf, the C-terminal amino acid is a proline which, depending on the way that it is orientated, win either point a fusion away or into the body of the molecule. There is also a proline near the N-terminus. In one aspect of the invention, the proline at the N- and/or the C-termini may be changed out. In another aspect of the invention, the C-terminal disulfide bond may be eliminated to untether the C-terminus.

GLP-1 Therapeutic Proteins and Peptides

In mammals, the glucagon gene encodes the precursor proglucagon which is processed to yield a tissue-determined variety of peptide products. Specifically, tissue-specific processing of proglucagon gives rise to glucagon in the brain, and glicentin, oxyntomodulin, and glucagon-like peptide-1 (GLP-1). and glucagon-like peptide-2 (GLP-2) in the intestine. The organization of these peptides in the proglucagon precursor was elucidated by the molecular cloning of cDNAs from rat, hamster, and bovine pancreas. Analyses of the proglucagon precursor indicate that the GLP-1 and GLP-2 peptides are separated from each other by a spacer or intervening peptide.

Glucagon-Like Peptide-1 (GLP-1) is a gastrointestinal hormone that regulates insulin secretion belonging to the so-called enteroinsular axis. The enteroinsular axis designates a group of hormones, released from the gastrointestinal mucosa in response to the presence and absorption of nutrients in the gut, which promote an early and potentiated release of insulin. The incretin effect which is the enhancing effect on insulin secretion is probably essential for a normal glucose tolerance. GLP-1 is a physiologically important insulinotropic hormone because it is responsible for the incretin effect.

GLP-1 is a product of proglucagon (Bell, et al., Nature, 1983, 304: 368-371). It is synthesized in intestinal endocrine cells in two principal major molecular forms, as GLP-1(7-36)amide and GLP-1(7-37). The peptide was first identified following the cloning of cDNAs and genes for proglucagon in the early 1980s.

Initial studies done on the full length peptide GLP-1(1-37) and GLP-1 (1-36^{amide}) concluded that the larger GLP-1 molecules are devoid of biological activity. In 1987, three independent research groups demonstrated that removal of the first six amino acids resulted in a GLP-1 molecule with enhanced biological activity.

The amino acid sequence of GLP-1 is disclosed by Schmidt et al. (1985 Diabetologia 28 704-707). Human GLP-1 is a 37 amino acid residue peptide originating from preproglucagon which is synthesized in the L-cells in the distal ileum, in the pancreas, and in the brain. Processing of preproglucagon to GLP-1(7-36^{amide}), GLP-1(7-37) and GLP-2 occurs mainly in the L-cells. The amino acid sequence of GLP-1(7-36^{amide}) and GLP-1(7-37) is (SEQ ID NO: 6):

His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-X

wherein X is NH₂ for GLP-1(7-36^{amide}) and X is Gly for GLP-1(7-37). Accordingly, A8 of GLP-1(7-36) and (7-37) corresponds to A2 of SEQ ID NO.: 6, K34 of GLP-1(7-36) and (7-37) corresponds to K28, etc.

GLP-1 like molecules possesses anti-diabetic activity in human subjects suffering from Type II (non-insulin-dependent diabetes mellitus (NIDDM)) and, in some cases, even Type I diabetes. Treatment with GLP-1 elicits activity, such as increased insulin secretion and biosynthesis, reduced glucagon secretion, delayed gastric emptying, only at elevated glucose levels, and thus provides a potentially much safer therapy than insulin or sulfonylureas. Post-prandial and glucose levels in patients can be moved toward normal levels with proper GLP-1 therapy. There are also reports suggesting GLP-1-like molecules possess the ability to preserve and even restore pancreatic beta cell function in Type-II patients.

Any GLP-1 sequence may be used to make Tf fusion proteins of the present invention, including GLP-1(7-35), GLP-1(7-36), and GLP-1(7-37). GLP-1 also has powerful actions on the gastrointestinal tract. Infused in physiological amounts, GLP-1 potently inhibits pentagastrin-induced as well as meal-induced gastric acid secretion (Schjoldager et al., Dig. Dis. Sci. 1989, 35:703-708; Wettergren et al., Dig Dis Sci 1993; 38:665-673). It also inhibits gastric emptying rate and pancreatic enzyme secretion (Wettergren et al., Dig Dis Sci 1993; 38:665-673). Similar inhibitory effects on gastric and pancreatic secretion and motility may be elicited in humans upon perfusion of the ileum with carbohydrate- or lipid-containing solutions (Layer et al., Dig Dis Sci 1995, 40:1074-1082; Layer et al., Digestion 1993, 54: 385-38). Concomitantly, GLP-1 secretion is greatly stimulated, and it has been speculated that GLP-1 may be at least partly responsible for this so-called “ileal-brake” effect (Layer et al., Digestion 1993; 54: 385-38). In fact, recent studies suggest that, physiologically, the ileal-brake effects of GLP-1 may be more important than its effects on the pancreatic islets. Thus, in dose response studies GLP-1 influences gastric emptying rate at infusion rates at least as low as those required to influence islet secretion (Nauck et al., Gut 1995; 37 (suppl. 2): A124).

GLP-1 seems to have an effect on food intake. Intraventricular administration of GLP-1 profoundly inhibits food intake in rats (Schick et al. in Ditschuneit et al. (eds.). Obesity in Europe, John Libbey & Company ltd, 1994: pp. 363-367: Turton et a., Nature 1996, 379: 69-72). This effect seems to be highly specific. Thus, N-terminally extended GLP-1(PG 72-107) amide is inactive and appropriate doses of the GLP-1 antagonist, exendin 9-39, abolish the effects of GLP-1(Tang-Christensen et al., Am. J. Physiol., 1996, 271(4 Pt 2):R848-56). Acute, peripheral administration of GLP-1 does not inhibit food intake acutely in rats (Tang-Christensen et al., Am. J. Physiol., 1996, 271(4 Pt 2):R848-56; Turton et al., Nature 1996, 379: 69-72). However, it remains possible that GLP-1 secreted from the intestinal L-cells may also act as a satiety signal.

In diabetic patients, GLP-1's insulinotropic effects and the effects of GLP-1 on the gastrointestinal tract are preserved (Willms et al, Diabetologia 1994; 37, suppl. 1: A118), which may help curtail meal-induced glucose excursions, but, more importantly, may also influence food intake. Administered intravenously, continuously for one week, GLP-1 at 4 ng/kg/min has been demonstrated to dramatically improve glycaemic control in NIDDM patients without significant side effects (Larsen et al., Diabetes 1996; 45, suppl. 2: 233A.).

GLP-1/transferrin fusion proteins comprising at least one analog of GLP-1 and fragments thereof are useful in the treatment of Type 1 and Type 2 diabetes and obesity.

Administration of GLP-1 and fragments thereof may also be useful for the treatment of congestive heart failure and non-alcoholic, non-fatty liver disease.

As used herein, the tenn “GLP-1 molecule” means GLP-1, a GLP-1 analog, or GLP-1 derivative.

As used herein, the term “GLP-1 analog” is defined as a molecule having one or more amino acid substitutions, deletions, inversions, or additions compared with GLP-1. Many GLP-1 analogs are known in the art and include, for example, GLP-1(7-34), GLP-1(7-35), GLP-1(7-36), Val⁸-GLP-1(7-37), Gly⁸-GLP-1(7-37), Ser⁸-GLP-1(7-37), Gln⁹-GLP1(7-37), D-Gln⁹-GLP-1(7-37), Thr¹⁶-Lys¹⁸-GLP-1(7-37), and Lys¹⁸-GLP-1(7-37)(see SEQ ID NO: 96). A preferred analog comprises GLP-1 (7-37; A8X,K34X), wherein X is any amino acid other than the native GLP-1 sequence, or GLP-1 (7-37; A8G,K34A). Other analogs include dipeptidyl-peptidase resistant versions of GLP-1, wherein the N-terminal end of the peptide is protected. Such analogs include, but are not limited to GLP-1 with additional amino acids, such as histidine residue added to the N-terminal end or substituted into the N-terminal amino acids (amino acid 7 or 8 in GLP-1(7-36) or GLP-1(7-37) which corresponds to amino acid 1 or 2 in SEQ ID NO.: 6). In these analogs, the N-terminal end may comprise the residues His-His-Ala, Gly-His-Ala, His-Gly-Glu, His-Ser-Glu, His-Ala-Glu, His-Gly-Glu, His-Ser-Glu, His-His-Ala-Glu, His-His-Gly-Glu, His-His-Ser-Glu, Gly-His-Ala-Glu, Gly-His-Gly-Glu, Gly-His-Ser-Glu (see SEQ ID NOS: 77-82, respectively), His-X-Ala-Glu, His-X-Gly-Glu, His-X-Ser-Glu, wherein X is any amino acid. U.S. Pat. No. 5,118,666 discloses examples of GLP-1 analogs such as GLP-1(7-34) and GLP-1(7-35).

In another embodiment, the GLP-1 peptide has mutations to make it less susceptible to cleavage by neutral endopeptidase (NEP). The inventors of the present invention have found that BRX0585 (SEQ ID NO.: 12) is less susceptible to NEP cleavage compared to an unfused GLP-1 protein.

Several mutations have the potential ofdisrupting NEP cleavage of GLP-1. For instance, mutations at or near the N-terminal side of hydrophobic amino acids can reduce the ability of NEP to cleave GLP-1. GLP-1(7-36) and GLP-1(7-37) are rich in hydrophobic amino acids from E27 to L32 (corresponds to E21 to L26 of SEQ ID NO.: 6). The sites most susceptible to NEP cleavage within GLP-1 are E27↓F28 (corresponds to E21F22 and W25↓L26 of SEQ ID NO.: 6) and W31↓L32 (corresponds to E21 to L26 of SEQ ID NO.: 6).

However, it should be noted that residue F28 and L32 may be important for receptor binding. A loss of receptor binding is acceptable if the ability of GLP-1 to activate the receptor is maintained. For instance, A30Q appears to result in a loss of receptor binding but an increase in receptor activation. According, the present invention includes mutating one or more hydrophobic amino acids and/or amino acids in the E27 to L32 region to disrupt NEP cleavage while maintaining GLP-1 receptor activation. The invention also includes mutating amino acids near, i.e., preferably within 5 amino acids, of hydrophobic amino acids and/or amino acids in the E27 to L32 region to disrupt NEP cleavage while maintaining GLP-1 receptor activation. For instance, one or more mutations selected from the group consisting of E27X, F28X, I29X, A30Q, W31X, L32X, and/or V33X can be used to disrupt NEP cleavage, wherein X is any amino acid other than glycine (G), proline (P), or cystine (C). In one embodiment, the mutation is A30Q (corresponds to A24Q of SEQ ID NO.: 6). See Hupe-Sodmann et al., 1995, Regulatory Peptides. 58: 149-156 and Hupe-Sodmann et al., 1997, Peptides. 18: 625-632. In another embodiment, the invention includes the use of the C-terminus of Exendin 4 or a derivative thereof (CEx; SEQ ID NO.: 15) to reduce susceptibility to NEP.

The term “GLP-1 derivative” is defined as a molecule having tile amino acid sequence of GLP-1 or a GLP-1 analog, but additionally having chemical modification of one or more of its amino acid side groups, α-carbon atoms, terminal amino group, or terminal carboxylic acid group. A chemical modification includes, but is not limited to, adding chemical moieties, creating new bonds, and removing chemical moieties.

As used herein, the term “GLP-1 related compound” refers to any compound falling within the GLP-1 , GLP-1 analog, or GLP-1 derivative definition.

WO 91/11457 discloses analogs of the active GLP-1 peptides 7-34, 7-35, 7-36, and 7-37 which can also be useful as GLP-1 moieties.

EP 0708179-A2 (Eli Lilly & Co.) discloses GLP-1 analogs and derivatives that include an N-terminal imidazole group and optionally an unbranched C₆-C₁₀ acyl group in attached to the lysine residue in position 34.

EP 0699686-A2 (Eli Lilly & Co.) discloses certain N-terminal truncated fragments of GLP-1 that are reported to be biologically active.

U.S. Pat. No. 5,545,618 discloses GLP-1 molecules consisting essentially of GLP-1(7-34), GLP1(7-35), GLP-1(7-36), or GLP-1(7-37), or the amide forms thereof, and pharmaceutically-acceptable salts thereof, having at least one modification selected from the group consisting of: (a) substitution of glycine,serine, cysteine, threonine, asparagine, glutamine, tyrosine, alaninie, valine, isoleucine, leucine, methionine, phenylalanine, arginine, or D-lysine for lysine at position 26 and/or position 34; or substitution of glycine, serine, cysteine, threonine, asparagine, glutamine, tyrosine, alanine, valine, isoleucine, leucine, methionine, phenylalaninie, lysine, or a D-arginine for arginine at position 36; (b) substitution of an oxidation-resistant amino acid for tryptophan at position 31; (c) substitution of at least one of: tyrosine for valine at position 16; lysine for serine at position 18; aspartic acid for glutamic acid at position 21; serine for glycine at position 22; arginine for glutamine at position 23; arginine for alanine at position 24; and glutamine for lysine at position 26; and (d) substitution of at least one of: glycine, serine, or cysteine for alanine at position 8; aspartic acid, glycine, serine, cysteine, threonine, asparagine, glutamine, tyrosine, alanine, valine, isoleucine, leucine, methionine, or phenylalanine for glutamic acid at position 9; serine, cysteine, threonine, asparagine, glutamine, tyrosine, alanine, valine, isoleucine, leucine, methionine, or phenylalanine for glycine at position 10; and glutamic acid for aspartic acid at position 15; and (e) substitution of glycine, serine, cysteine, threonine, asparagine, glutamine, tyrosine, alanine, valine, isoleucine, leucine, methionine, or phenylalanine, or the D- or N-acylated or alkylated form of histidine for histidine at position 7 (see SEQ ID NOS: 83-87, respectively); wherein, in the substitutions is (a), (b), (d), and (e), the substituted amino acids can optionally be in the D-form and the amino acids substituted at position 7 can optionally be in the N-acylated or N-alkylated form.

U.S. Pat. No. 5,118,666 discloses a GLP-1 molecule having insulinotropic activity. Such molecule is selected from the group consisting of a peptide having the amino acid sequence His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys (SEQ ID NO: 7) or His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly (SEQ ID NO: 8); and a derivative of said peptide and wherein said peptide is selected from the group consisting of: a pharmaceutically-acceptable acid addition salt of said peptide; a pharmaceutically-acceptable carboxylate salt of said peptide; a pharmaceutically-acceptable lower alkylester of said peptide; and a pharmaceutically-acceptable amide of said peptide selected from the group consisting of amide, lower alkyl amide, and lower dialkyl amide.

U.S. Pat. No. 6,277,819 teaches a method of reducing mortality and morbidity after myocardial infarction comprising administering GLP-1, GLP-1 analogs, and GLP-1 derivatives to the patient. The GLP-1 analog being represented by the following structural formula (SEQ ID NO: 9): R₁-X₁-Glu-Gly-Thr-Phe-Thr-Ser-Asp-Val-Ser-Ser-Tyr-Leu-X₂-Gly-Gln-Ala-Ala-Lys-X₃-Phe-Ile-Ala-Trp-Leu-Val-Lys-Gly-Arg-R₂ and pharmaceutically-acceptable salts thereof, wherein: R₁ is selected from the group consisting of L-histidine, D-histidine, desaminio-histidine, 2-amino-histidine, .beta.-hydroxy-histidine, homohistidine, alpha-fluoromethyl-histidine, and alpha-methyl-histidine; X₁ is selected from the group consisting of Ala, Gly, Val, Thr, Ile, and alpha-methyl-Ala; X₂ is selected from the group consisting of Glu, Gin, Ala, Thr, Ser, and Gly; X₃ is selected from the group consisting of Glu, Gln, Ala, Thr, Ser, and Gly; R₂ is selected from the group consisting of NH₂, and Gly—OH; provided that the GLP-1 analog has an isoelectric point in the range from about 6.0 to about 9.0 and further providing that when R₁ is His, X₁ is Ala, X₂ is Glu, and X₃ is Glu, R₂ must be NH₂.

Ritzel et al. (Journal of Endocrinology. 1998. 159: 93-102) disclose a GLP-1 analog, [Ser⁸]GLP-1 in which the N-terminal second amino acid, alanine, is replaced with serine. The modification did not impair the insulinotropic action of the peptide but produced an analog with increased plasma stability as compared to GLP-1.

U.S. Pat. No. 6,429,197 teaches that GLP-1 treatment after acute stroke or hemorrhage, preferably intravenous administration, can be an ideal treatment because it provides a means for optimizing insulin secretion, increasing brain anabolism, enhancing insulin effectiveness by suppressing glucagon, and maintaining euglycemia or mild hypoglycemia with no risk of severe hypoglycemia or other adverse side effects. The present invention provides a method of treating the ischemic or reperfused brain with GLP-1 or its biologically active analogues after acute stroke or hemorrhage to optimize insulin secretion, to enhance insulin effectiveness by suppressing glucagon antagonism, and to maintain euglycemia or mild hypoglycemia with no risk of severe hypoglycemia.

U.S. Pat. No. 6,277,819 provides a method of reducing mortality and morbidity after myocardial infarction, comprising administering to a patient in need thereof, a compound selected from the group consisting of GLP-1, GLP-1 analogs, GLP-1 derivatives and pharmaceutically-acceptable salts thereof, at a dose effective to normalize blood glucose.

U.S. Pat. Nos. 6,191,102 and 6,583,111 disclose methods of reducing body weight in a subject in need of body weight reduction by administering to the subject a composition comprising a glucagon-like peptide-1 (GLP-1), a glucagon-like peptide analog (GLP-1 analog), a glucagon-like peptide derivative (GLP-1 derivative) or a pharmaceutically acceptable salt thereof in a dose sufficient to cause reduction in body weight for a period of time effective to produce weight loss, said time being at least 4 weeks.

GLP-1 is fully active after subcutaneous administration (Ritzel et al., Diabetologia 1995: 38: 720-725), but is rapidly degraded mainly due to degradation by dipeptidyl peptidase IV-like enzymes (Deacon et al., J. Clin Endocrinol Metab 1995, 80: 952-957: Deacon et al., 1995. Diabetes 44: 1126-1131). Thus, unfortunately, GLP-1 and many of its analogues have a short plasma half-life in humans (Orskov et al., Diabetes 1993; 42:658-661). Accordingly, it is an objective of the present invention to provide transferrin fusion proteins comprising GLP-1 or analogues thereof which have a protracted profile of action relative to GLP-1(7-37). It is a further object of the invention to provide derivatives of GLP-1 and analogues thereof which have a lower clearance than GLP-1(7-37). Moreover, it is an object of the invention to provide pharmaceutical compositions comprising GLP-1/transferrin fusion proteins or GLP-1 analog/transferrin fusion proteins with improved stability. Additionally, the present invention includes the use of GLP-1/transferrin fusion proteins or GLP-1 analog/transferrin fusion proteins to treat diseases such as but not limited to type II diabetes, obesity, metabolic syndrome, pre-diabetes, non-fatty liver disease or congestive heart failure and those described above.

Any GLP-1 therapeutic molecule may be used as the fusion partner to Tf according to the methods and compositions of the present invention. As used herein, a “therapeutic molecule” is GLP-1 or variant or analog thereof capable of exerting a beneficial biological effect in vitro or in vivo. For instance, a beneficial effect as related to a disease state includes any effect that is advantageous to the treated subject, including disease prevention, disease stabilization, the lessening or alleviation of disease symptoms or a modulation, alleviation or cure of the underlying defect to produce an effect beneficial to the treated subject.

A modified transferrin fusion protein of the invention includes at least a fragment or variant of a therapeutic protein and at least a fragment or variant of modified serum transferrin, which are associated with one another, preferably by genetic fusion.

In further embodiments, a modified transferrin fusion protein of the invention may contain at least a fragment or variant of a GLP-1 therapeutic protein. In a further embodiment, the transferrin fusion proteins can contain peptide fragments or peptide variants of proteins wherein the variant or fragment retains at least one biological or therapeutic activity. The transferrin fusion proteins can contain GLP-1 therapeutic proteins that can be peptide fragments or peptide variants at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, or at least about 31 amino acids in length fused to the N and/or C termini, inserted within, or inserted into a loop of a modified transferrin.

The modified transferrin fusion proteins of the present invention may contain one or more peptides. Increasing the number of peptides may enhance the function of the peptides fused to transferrin and the function of the entire transferrin fusion protein. The peptides may be used to make a bi- or multi-functional fusion protein by including peptide or protein domains with multiple functions. For instance, a multi-functional fusion protein can be made with a GLP-1 therapeutic protein and a second protein to target the fusion protein to one or more specific targets.

The transferrin fusion of the present invention may comprise a linker linking the transferrin to the GLP-1 therapeutic peptide. Preferably, the linker is GLP-2 or the sequence PEAPTD (SEQ ID NO.: 13) in one or more copies. There are one or more GLP-1 peptides at the amino terminus of the fusion protein.

In another embodiment, the modified transferrin fusion molecules contain a GLP-1 therapeutic protein portion that can be fragments of a GLP-1 therapeutic protein that include the full length protein as well as polypeptides having one or more residues deleted from the amino terminus of the amino acid sequence.

In another embodiment, the modified transferrin fusion molecules contain a GLP-1 therapeutic protein portion that can be fragments of a GLP-1 therapeutic protein that include the full length protein as well as polypeptides having one or more residues deleted from the carboxy terminus of the amino acid sequence.

In another embodiment, the modified transferrin fusion molecules contain a GLP-1 therapeutic protein portion that can have one or more amino acids deleted from both the amino and the carboxy termini.

In another embodiment, the modified transferrin fusion molecules contain a GLP-1 therapeutic protein portion that is at least about 80%, 85%, 90%, 95%, 96%, 97%, 98% or 99% identical to a reference GLP-1 therapeutic protein set forth herein, or fragments thereof. In further embodiments, the transferrin fusion molecules contain a GLP-1 therapeutic protein portion that is at least about 80%, 85%, 90%, 95%, 96%, 97%, 98% or 99% identical to reference polypeptides having the amino acid sequence of N- and C-terminal deletions as described above.

In another embodiment, the modified transferrin fusion molecules contain the therapeutic protein portion that is at least about 80%, 85%, 90%, 95%, 96%, 97%, 98%, 99% or 100%, identical to, for example, the native or wild-type amino acid sequence of a GLP-1 therapeutic protein. Fragments, of these polypeptides are also provided.

The GLP-1 therapeutic proteins corresponding to a GLP-1 therapeutic protein portion of a modified transferrin fusion protein of the invention, such as cell surface and secretory proteins, can be modified by the attachment of one or more oligosaccharide groups. The modification referred to as glycosylation can significantly affect the physical properties of proteins and can be important in protein stability, secretion, and localization. Glycosylation occurs at specific locations along the polypeptide backbone. There are usually two major types of glycosylation: glycosylation characterized by O-linked oligosaccharides, which are attached to serine or threonine residues; and glycosylation characterized by N-linked oligosaccharides, which are attached to asparagine residues in an Asn-X-Ser/Thr sequence, where X can be an amino acid except proline. Variables such as protein structure and cell type influence the number and nature of the carbohydrate units within the chains at different glycosylation sites. Glycosylation isomers are also common at the same site within a given cell type. For example, several types of human interferon are glycosylated.

Therapeutic proteins corresponding to a therapeutic protein portion of a transferrin fusion protein of the invention, as well as analogs and variants thereof, may be modified so that glycosylation at one or more sites is altered as a result of manipulation(s) of their nucleic acid sequence by the host cell in which they are expressed, or due to other conditions of their expression. For example, glycosylation isomers may be produced by abolishing or introducing glycosylation sites, e.g., by substitution or deletion of amino acid residues, such as substitution of glutamine for asparagine, or unglycosylated recombinant proteins may be produced by expressing the proteins in host cells that win not glycosylate them, e.g. in glycosylation-deficient yeast. These approaches are known in the art.

Therapeutic proteins and their nucleic acid sequences are well known in the art and available in public databases such as Chemical Abstracts Services Databases (e.g., the CAS Registry), GenBank, and GenSeq. The Accession Numbers and sequences referred to below are herein incorporated by reference in their entirety.

In other embodiments, the transferrin fusion proteins of the invention are capable of a therapeutic activity and/or biologic activity, corresponding to the therapeutic activity and/or biologic activity of the therapeutic protein described elsewhere in this application. In further embodiments, the therapeutically active protein portions of the transferrin fusion proteins of the invention are fragments or variants of the reference sequences cited herein.

The present invention is further directed to modified Tf fusion proteins comprising fragments of the GLP-1 therapeutic proteins herein described. Even if deletion of one or more amino acids from the N-terminus of a protein results in modification or loss of one or more biological functions of the therapeutic protein portion, other therapeutic activities and/or functional activities (e.g., biological activities, ability to multimerize, ability to bind a ligand) may still be retained. For example, the ability of polypeptides with N-terminal deletions to induce and/or bind to antibodies which recognize the complete or mature forms of the polypeptides generally will be retained with less than the majority of the residues of the complete polypeptide removed from the N-terminus. Whether a particular polypeptide lacking N-terminal residues of a complete polypeptide retains such immunologic activities can be assayed by routine methods described herein and otherwise known in the art. It is not unlikely that a mutant with a large number of deleted N-terminal amino acid residues may retain some biological or immunogenic activities. In fact, peptides composed of as few as six amino acid residues may often evoke an immune response.

Also as mentioned above, even if deletion of one or more amino acids from the N-terminus or C-terminus of a therapeutic protein results in modification or loss of one or more biological functions of the protein, other functional activities (e.g., biological activities, ability to multimerize, ability to bind a ligand) and/or therapeutic activities may still be retained. For example the ability of polypeptides with C-terminal deletions to induce and/or bind to antibodies which recognize the complete or mature forms of the polypeptide generally win be retained when less than the majority of the residues of the complete or mature polypeptide are removed from the C-terminus. Whether a particular polypeptide lacking the N-terminal and/or, C-terminal residues of a reference polypeptide retains therapeutic activity can readily be determined by routine methods described herein and/or otherwise known in the art.

Peptide fragments of the GLP-1 therapeutic proteins can be fragments comprising, or alternatively, consisting of, an amino acid sequence that displays a therapeutic activity and/or functional activity (e.g. biological activity) of the polypeptide sequence of the therapeutic protein of which the amino acid sequence is a fragment.

The peptide fragments of the GLP-1 therapeutic protein may comprise only the N- and C-termini of the proteins i.e., the central portion of the therapeutic protein has been deleted. Alternatively, the peptide fragments may comprise non-adjacent and/or adjacent portions of the central part of the therapeutic protein.

Other polypeptide fragments are biologically active fragments. Biologically active fragments are those exhibiting activity similar, but not necessarily identical, to an activity of a therapeutic protein used in the present invention. The biological activity of the fragments may include an improved desired activity, or a decreased undesirable activity.

Generally, variants of proteins are overall very similar, and, in many regions, identical to the amino acid sequence of the therapeutic protein corresponding to a GLP-1 therapeutic protein portion of a transferrin fusion protein of the invention. Nucleic acids encoding these variants are also encompassed by the invention.

Further therapeutic polypeptides that may be used in the invention are polypeptides encoded by polynucleotides which hybridize to the complement of a nucleic acid molecule encoding an amino acid sequence of a GLP-1 therapeutic protein under stringent hybridization conditions which are known to those of skin in the art. (see, for example, Ausubel, F. M. et al., eds., 1989 Current protocol in Molecular Biology, Green Publishing Associates, Inc., and John Wiley & Sons Inc., New. York). Polynucleotides encoding these polypeptides are also encompassed by the invention.

By a polypeptide-having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence, or in one or more contiguous groups within the reference sequence.

As a practical matter, whether any particular polypeptide is at least about 80%, 85%, 90%, 95%, 96%, 97%, 98% or 99% identical to, for instance, the amino acid sequence of a transferrin fusion protein of the invention or a fragment thereof (such, as the therapeutic protein portion of the transferrin fusion protein or the transferrin portion of the transferrin fusion protein), can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brufiag et al. (Comp. App. Biosci 245 (1990)).

The polynucleotide variants of the invention may contain alterations in the coding regions, non-coding regions, or both. Polynucleotide variants containing alterations which produce silent substitutions, additions, or deletions, but do not alter the properties or activities of the encoded polypeptide may be used to produce modified Tf fusion proteins. Nucleotide variants produced by silent substitutions due to the degeneracy of the genetic code can be utilized. Moreover, polypeptide variants in which less than about 50, less than 40, less than 30, less than 20, less than 10, or 5-50, 5-25, 5-10, 1-5, or 1-2 amino acids are substituted, deleted, or added in any combination can also be utilized. Polynucleotide variants can be produced for a variety of reasons, e.g., to optimize codon expression for a particular host (change codons in the human mRNA to those preferred by a host, such as, yeast or E. coli as described above).

In other embodiments, the GLP-1 therapeutic protein moiety has conservative substitutions compared to the wild-type sequence. By “conservative substitutions” is intended swaps within groups such as replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly. Guidance concerning how to make phenotypically silent amino acid substitutions is provided, for example, in Bowie et al., “Deciphering the Message in Protein Sequences: Tolerance to Amino Acid Substitutions,” Science 247: 1306-1310 (1990). In specific embodiments, the polypeptides of the invention comprise, or alternatively, consist of, fragments or variants of the amino acid sequence of a therapeutic protein described herein and/or serum transferrin, and/ modified transferrin protein of the invention, wherein the fragments or variants have 1-5, 5-10, 5-25, 5-50, 10-50 or 50-150 amino acid residue additions, substitutions, and/or deletions when compared to the reference amino acid sequence. In further embodiments, the amino acid substitutions are conservative. Nucleic acids encoding these polypeptides are also encompassed by the invention.

The modified fusion proteins of the present invention can be composed of amino-acids joined to each other by peptide bonds or modified peptide bonds and may contain amino acids other than the 20 gene-encoded amino acids. The polypeptides may be modified by either natural processes, such as post-translational processing, or by chemical modification techniques which are well known in the art. Such modifications are well described in basic texts and in more detailed monographs, as well as in a voluminous research literature.

Modifications can occur anywhere in a polypeptide, including the peptide backbone, the amino acid side-chains and the amino or carboxy termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, glycosylation, GPI anchor formation, hydroxylation, iodination, methylation, myristylation, oxidation, pegylation, proteolytic processing, phosphorylation, prenylation, racemization, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifter et al. (1990) Meth. Enzymol. 182:626-646; Rattan et al., Ann. N.Y. Acad. Sci. 663:48-62

GLP-2

GLP-2 is a 33 amino acid peptide that is expressed in a tissue specific manner from the glucagon gene. GLP-2 has the following sequence: His-Ala-Asp-Gly-Ser-Phe-Ser-Asp-Glu-Met-Asn-Thr-Ile-Leu-Asp-Asn-Leu-Ala-Thr-Arg-Asp-Phe-Ile-Asn-Trp-Leu-Ile-Gln-Thr-Lys-Ile-Thr-Asp-Arg, SEQ ID NO: 96). GLP-2 is related to GLP-1. However, unlike GLP-1 , the physiological role of GLP-2 is unknown.

In the present invention, GLP-2 is used as a linker to link GLP-1 to transferrin or modified transferrin. The inventors have found that using GLP-2 as a linker in the GLP/mTf fusion protein stabilizes the fusion protein producing a more potent GLP-1 peptide, presumably because the GLP-1 peptide is more available for binding to its receptor. Thus, the present invention provides a transferrin fusion protein comprising GLP-1 linked to GLP-2 which is linked to a transferrin or modified transferrin molecule. The transferrin molecule could be a serum transferrin, a lactoferrin, or a melanotransferrin molecule.

There is at least one GLP-1 molecule at the N-terminus of the transferrin fusion protein. In one embodiment there are two to five GLP-1 molecules. In another embodiment, there are three or four GLP-1 molecules.

In a preferred embodiment, the GLP-2 linker sequence is modified such that the amino acid corresponding to N-terminal amino acid of GLP-2, i.e. histidine, is either not present or is changed to another amino acid.

Nucleic Acids

The present invention also provides nucleic acid molecules encoding fusion proteins comprising a transferrin protein or a portion of a transferrin protein covalently linked or joined to a GLP-1 therapeutic protein, preferably a therapeutic protein. As discussed in more detail below, any therapeutic protein may be used. The fusion protein may further comprise a linker region, for instance a linker less than about 50, 40, 30, 20, or 10 amino acid residues. The linker can be covalently linked to and between the transferrin protein or portion thereof and the therapeutic protein, preferably the therapeutic protein. Nucleic acid molecules of the invention may be purified or not.

Host cells and vectors for replicating the nucleic acid molecules and for expressing the encoded fusion proteins are also provided. Any vector s or host cells may be used, whether prokaryotic or eukaryotic, but eukaryotic expression systems, in particular yeast expression systems, may be preferred. Many vectors and host cells are known in the art for such purposes. It is well within the skin of the art to select an appropriate set for the desired application.

DNA sequences encoding transferrin, portions of transferrin and GLP-1 and linker may be cloned from a variety of genomic or cDNA libraries known in the art. The techniques for isolating such DNA sequences using probe-based methods are conventional techniques and are well known to those skilled in the art. Probes for isolating such DNA sequences may be based on published DNA or protein sequences (see, for example, Baldwin, G. S. (1993) Comparison of Transferrin Sequences from Different Species. Comp. Biochem. Physiol. 106B/1:203-218 and all references cited therein, which are hereby incorporated by reference in their entirety). Alternatively, the polymerase chain reaction (PCR) method disclosed by Mullis et al. (U.S. Pat. No. 4,683,195) and Mullis (U.S. Pat. No. 4,683,202), incorporated herein by reference may be used. The choice of library and selection of probes for the isolation of such DNA sequences is within the level of ordinary skin in the art.

As known in the art “similarity” between two polynucleotides or polypeptides is determined by comparing the nucleotide or amino acid sequence and its conserved nucleotide or amino acid substitutes of one polynucleotide or polypeptide to the sequence of a second polynucleotide or polypeptide. Also known in the art is “identity” which means the degree of sequence relatedness between two polypeptide or two polynucleotide sequences as determined by the identity of the match between two strings of such sequences. Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

While there exist a number of methods to measure identity and similarity between two polynucleotide or polypeptide sequences, the terms “identity” and “similarity” are well known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., SIAM J. Applied Math., 48: 1073 (1988). Methods commonly employed to determine identity or similarity between two sequences include, but are not limited to those disclosed in Guide to Huge Computers, Martin J. Bishop, ed., Academic Press, San Diego, 1994, and Carillo, H., and Lipman, D., SIAM J. Applied Math. 48:1073 (1988).

Preferred methods to determine identity are designed to give the largest match between the two sequences tested. Methods to determine identity and similarity are codified in computer programs. Preferred computer program methods to determine identity and similarity between two sequences include, but are not limited to, GCG program package (Devereux, et al., Nucl. Acid Res. 12(1):387 (1984)), BLASTP, BLASTN, FASTA (Atschul, et al., J. Mol. Biol. 215:403 (1990)). The degree of similarity or identity referred to above is determined as the degree of identity between the two sequences, often indicating a derivation of the first sequence from the second. The degree of identity between two nucleic acid sequences may be determined by means of computer programs known in the art such as GAP provided in the GCG program package (Needleman and Wunsch J. Mol. Biol. 48:443-453 (1970)). For purposes of determining the degree of identity between two nucleic acid sequences for the present invention, GAP is used with the following settings: GAP creation penalty of 5.0 and GAP extension penalty of 0.3.

Codon Optimization

The degeneracy of the genetic code permits variations of the nucleotide sequence of a transferrin protein and/or therapeutic protein of interest, while still producing a polypeptide having the identical amino acid sequence as the polypeptide encoded by the native DNA sequence. The procedure, known as “codon optimization” (described in U.S. Pat. No. 5,547,871 which is incorporated herein by reference in its entirety) provides one with a means of designing such an altered DNA sequence. The design of codon optimized genes should take into account a variety of factors, including the frequency of codon usage in an organism, nearest neighbor frequencies, RNA stability, the potential for secondary structure formation, the route of synthesis and the intended future DNA manipulations of that gene. In particular, available methods may be used to alter the codons encoding a given fusion protein with those most readily recognized by yeast when yeast expression systems are used.

The degeneracy of the genetic code permits the same amino acid sequence to be encoded and translated in many different ways. For example, leucine, serine and arginine are each encoded by six different codons, while valine, proline, threonine, alanine and glycine are each encoded by four different codons. However, the frequency of use of such synonymous codons varies from genome to genome among eukaryotes and prokaryotes. For example, synonymous codon-choice patterns among mammals are very similar, while evolutionarily distant organisms such as yeast (such as S. cerevisiae), bacteria (such as E. coli) and insects (such as D. melanogaster) reveal a clearly different pattern of genomic codon use frequencies (Grantham, R., et al., Nucl. Acid Res., 8, 49-62 (1980); Grantham, R., et al., Nucl. Acid Res., 9, 43-74 (1981); Maroyama, T., et al., Nucl. Acid Res., 14, 151-197 (1986); Aota, S., et al., Nucl. Acid Res., 16, 315-402 (1988); Wada, K., et al., Nucl. Acid Res., 19 Supp., 1981-1985 (1991); Kurland, C. G., FEBS Lett., 285, 165-169 (1991)). These differences in codon-choice patterns appear to contribute to the overall expression levels of individual genes by modulating peptide elongation rates. (Kurland, C. G., FEBS Lett., 285, 165-169 (1991); Pedersen, S., EMBO J., 3, 2895-2898 (1984); Sorensen, M. A., J. Mol. Biol., 207, 365-377 (1989); Randall, L. L., et al., Eur. J. Biochem., 107, 375-379 (1980); Curran, J. F., and Yarus, M., J. Mol. Biol., 209, 65-77 (1989); Varenne, S., et al., J. Mol. Biol., 180, 549-576 (1984), Varenne, S., et al., J. Mol, Biol., 180, 549-576 (1984); Garel, J.-P., J. Theor. Biol., 43, 211-225 (1974); Ikemura, T., J. Mol. Biol., 146, 1-21 (1981); Ikemura, T., J. Mol. Biol., 151, 389-409 (1981)).

The preferred codon usage frequencies for a synthetic gene should reflect the codon usages of nuclear genes derived from the exact (or as closely related as possible) genome of the cell/organism that is intended to be used for recombinant protein expression, particularly that of yeast species. As discussed above, in one preferred embodiment the human Tf sequence is codon optimized, before or after modification as herein described for yeast expression as may be the therapeutic protein nucleotide sequence(s).

Vectors

Expression units for use in the present invention win generally comprise the following elements, operably linked in a 5′ to 3′ orientation: a transcriptional promoter, a secretory signal sequence, a DNA sequence encoding a modified Tf fusion protein comprising transferrin protein or a portion of a transferrin protein joined to a DNA sequence encoding a linker, a GLP-1 and a transcriptional terminator. As discussed above, any arrangement of the GLP-1 and linker fused to the Tf portion may be used in the vectors of the invention. The selection of suitable promoters, signal sequences and terminators win be determined by the selected host cell and win be evident to one skilled in the art and are discussed more specifically below.

Suitable yeast vectors for use in the present invention are described in U.S. Pat. No. 6,291,212 and include YRp7 (Struhl el al., Proc. Natl. Acad. Sci. USA 76: 1035-1039, 1978), YEp13 (Broach et al., Gene 8: 121-133, 1979), pJDB249 and pJDB219 (Beggs, Nature 275:104-108, 1978), pPPC0005, pSeCHSA, pScNHSA, pC4 and derivatives thereof. Useful yeast plasmid vectors also include pRS403-406, pRS413-416 and the Pichia vectors available from Stratagene Cloning Systems, La Jolla, Calif. 92037, USA. Plasmids pRS403, pRS404, pRS405 and pRS406 are Yeast Integrating plasmids (Ylps) and incorporate the yeast selectable markers HIS3, TRP1, LEU2 and URA3. PlasmidspRS413˜41.6 are Yeast Centromere plasmids (YCps).

Such vectors win generally include a selectable marker, which may be one of any number of genes that exhibit a dominant phenotype for which a phenotypic assay exists to enable transformants to be selected. Preferred selectable markers are those that complement host cell auxotrophy, provide antibiotic resistance or enable a cell to utilize specific carbon sources, and include LEU2 (Broach el al. ibid.), URA3 (Botstein et al., Gene 8: 17, 1979), HIS3 (Struhl et al., ibid.) or POT1 (Kawasaki and Bell, EP 171,142). Other suitable selectable markers include the CAT gene, which confers chloramphenicol resistance on yeast cells. Preferred promoters for use in yeast include promoters from yeast glycolytic genes (Hitzeman et al., J Biol. Chem. 225: 12073-12080, 1980; Alber and Kawasaki, J. Mol. Appl. Genet. 1: 419-434, 1982; Kawasaki, U.S. Pat. No. 4,599,311) or alcohol dehydrogenase genes (Young et al., in Genetic Engineering of Microorganisms for Chemicals, Hollaender et al., (eds.), p. 355, Plenum, N.Y., 1982; Ammerer, Meth. Enzymol. 101: 192-201, 1983). In this regard, particularly preferred promoters are the TPI1 promoter (Kawasaki, U.S. Pat. No. 4,599,311) and the ADH2-4^{c} (see U.S. Pat. No. 6,291,212 promoter (Russell et al., Nature 304: 652-654, 1983). The expression units may also include a transcriptional terminator. A preferred transcriptional terminator is the TPI1 terminator (Alber and Kawasaki, ibid.). Other preferred vectors and preferred components such as promoters and terminators of a yeast expression system are disclosed in European Patents EP 0258067, EP 0286424, EP0317254, EP 0387319, EP 0386222, EP 0424117, EP 0431880, and EP 1002095; European Patent Publications EP 0828759, EP 0764209, EP 0749478, and EP 0889949; PCT Publication WO 00/44772 and WO 94/04687; and U.S. Pat. Nos. 5,739,007; 5,637,504; 5,302,697; 5,260,202; 5,667,986; 5,728,553; 5,783,423; 5,965,386; 6,150,133; 6,379,924; and 5,714,377; which are herein incorporated by reference in their entirety.

In addition to yeast, modified fusion proteins of the present invention can be expressed in filamentous fungi, for example, strains of the fungi Aspergillus. Examples of useful promoters include those derived from Aspergillus nidulans glycolytic genes, such as the adh3 promoter (McKnight et al., EMBO J. 4: 2093-2099, 1985) and the tpiA promoter. An example of a suitable terminator is the adh3 terminator (McKnight et al., ibid.). The expression units utilizing such components may be cloned into vectors that are capable of insertion into the chromosomal DNA of Aspergillus, for example.

Mammalian expression vectors for use in carrying out the present invention win include a promoter capable of directing the transcription of the modified Tf fusion protein. Preferred promoters include viral promoters and cellular promoters. Preferred viral promoters include the major late promoter from adenovirus 2 (Kaufman and Sharp, Mol. Cell. Biol. 2: 1304-13199, 1982) and the SV40 promoter (Subramani et al., Mol. Cell. Biol. 1: 854-864, 1981). Preferred cellular promoters include the mouse metallothionein 1 promoter (Palmiter et al., Science 222: 809-814, 1983) and a mouse V6 (see U.S. Pat. No. 6,291,212) promoter (Grant et al., Nuc. Acids Res. 15: 5496, 1987). A particularly preferred promoter is a mouse V_{H} (see U.S. Pat. No. 6,291,212) promoter (Loh et al., ibid.). Such expression vectors may also contain a set of RNA splice sites located downstream from the promoter and upstream from the DNA sequence encoding the transferrin fusion protein. Preferred RNA splice sites may be obtained from adenovirus and/or immunoglobulin genes.

Also contained in the expression vectors is a polyadenylation signal located downstream of the coding sequence of interest. Polyadenylation signals include the early or late polyadenylation signals from SV40 (Kaufman and Sharp, ibid.), the polyadenylation signal from the adenovirus 5 E1B region and the human growth hormone gene terminator (DeNoto et al., Nucl. Acid Res. 9: 3719-3730, 1981). A particularly preferred polyadenylation signal is the V_{H} (see U.S. Pat. No. 6,291,212) gene terminator (Loh et al., ibid.). The expression vectors may include a noncoding viral leader sequence, such as the adenovirus 2 tripartite leader, located between the promoter and the RNA splice sites. Preferred vectors may also include enhancer sequences, such as the SV40 enhancer and the mouse: (see U.S. Pat. No. 6,291,212) enhancer (Gillies, Cell 33: 717-728, 1983). Expression vectors may also include sequences encoding the adenovirus VA RNAs.

Transformation

Techniques for transforming fungi are well known in the literature, and have been described, for instance, by Beggs (ibid.), Hinnen et al. (Proc. Natl. Acad. Sci. USA 75: 1929-1933, 1978), Yelton et al., (Proc. Natl. Acad. Sci. USA 81: 1740-1747, 1984), and Russell (Nature 301: 167-169, 1983). Other techniques for introducing cloned DNA sequences into fungal cells, such as electroporation (Becker and Guarente, Methods in Enzymol. 194: 182-187, 1991) may be used. The genotype of the host cell win generally contain a genetic defect that is complemented by the selectable marker present on the expression vector. Choice of a particular host and selectable marker is well within the level of ordinary skill in the art.

Cloned DNA sequences comprising fusion proteins of the invention may be introduced into cultured mammalian cells by, for example, calcium phosphate-mediated transfection (Wigler et al., Cell 14: 725, 1978; Corsaro and Pearson, Somatic Cell Genetics 7: 603, 1981; Graham and Van der Eb, Virology 52: 456, 1973.) Other techniques for introducing cloned DNA sequences into mammalian cells, such as electroporation (Neumann et al., EMBO J. 1: 841-845, 1982), or lipofection may also be used. In order to identify cells that have integrated the cloned DNA, a selectable marker is generally introduced into the cells along with the gene or cDNA of interest. Preferred selectable markers for use in cultured mammalian cells include genes that confer resistance to drugs, such as neomycin, hygromycin, and methotrexate. The selectable marker may be an amplifiable selectable marker. A preferred amplifiable selectable marker is the DHFR gene. A particularly preferred amplifiable marker is the DHFR^{r} (see U.S. Pat. No. 6,291,212) cDNA (Simonsen and Levinson, Proc. Natl. Acad. Sci. USA 80: 2495-2499, 1983). Selectable markers are reviewed by Thilly (Mammalian Cell Technology, Butterworth Publishers, Stoneham, Mass.) and the choice of selectable markers is well within the level of ordinary skin in the art.

Host Cells

The present invention also includes a cell, preferably a yeast cell transformed to express a modified transferrin fusion protein of the invention. In addition to the transformed host cells themselves, the present invention also includes a culture of those cells, preferably a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away.

Host cells for use in practicing the present invention include eukaryotic cells, and in some cases prokaryotic cells, capable of being transformed or transfected with exogenous DNA and grown in culture, such as cultured mammalian, insect, fungal, plant and bacterial cells.

Fungal cells, including species of yeast (e.g., Saccharomyces spp., Schizosaccharomyces spp., Pichia spp.) may be used as host cells within the present invention. Examples of fungi including yeasts contemplated to be useful in the practice, of the present invention as hosts for expressing the, transferrin fusion protein of the inventions are Pichia (some species of which were formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Examples of Saccharomyces spp. are S. cerevisiae, S. italicus and S. rouxii. Examples of Kluyveromyces spp. are K. fragilis, K. lactis and K. marxianus. A suitable Torulaspora species is T. delbrueckii. Examples of Pichia spp. are P. angusta (formerly H. polymorpha) P. anomala (formerly H. anomala) and P. pastoris.

Particularly useful host cells to produce the Tf fusion proteins of the invention are the metlhylotrophic Pichia pastoris (Steinlein et al. (1995) Protein Express. Purif. 6:619-624). Pichia pastoris has been developed to be an outstanding host for the production of foreign proteins since its alcohol oxidase promoter was isolated and cloned: its transformation seas first reported in 1985. P. pastoris can utilize methanol as a carbon source in the absence of glucose. The P. pastoris expression system can use the methanol-induced alcohol oxidase (AOX1) promoter, which controls the gene that codes for the expression of alcohol oxidase, the enzyme which catalyzes the first step in the metabolism of methanol. This promoter has been characterized and incorporated into a series of P. pastoris expression vectors. Since the proteins produced in P. pastoris are typically folded correctly and secreted into the medium, the fermentation of genetically engineered P. pastoris provides an excellent alternative to E. coli expression systems. A number of proteins have been produced using this system, including tetanus toxin fragment, Bordatella pertussis pertactin, human serum albumin and lysozyne.

Strains of the yeast Saccharomyces cerevisiae are another preferred host. In a preferred embodiment, a yeast cell, or more specifically, a Saccharomyces cerevisiae host cell that contains a genetic deficiency in a gene required for asparagine-linked glycosylation of glycoproteins is used. S. cerevisiae host cells having such defects may be prepared using standard techniques of mutation and selection, although many available yeast strains have been modified to prevent or reduce glycosylation or hypermannosylation. Ballou et al. (J. Biol. Chem. 255: 5986-5991, 1980) have described the isolation of mannoprotein biosynthesis mutants that are defective in genes which affect asparagine-linked glycosylation. Gentzsch and Tanner (Glycobiology 7:481-486, 1997) have described a family of at least six genes (PMT1-6) encoding enzymes responsible for the first step in O-glycosylation of proteins in yeast. Mutants defective in one or more of these genes show reduced O-linked glycosylation and/or altered specificity of O-glycosylation.

In one embodiment, the host is a S. cerevisiae strain described in WO 05/061718, which is herein incorporated by reference in its entirety. For instance, the host can contain a pSAC35 based plasmid carrying a copy of the PDII gene or any other chaperone gene in a strain with the host version of PDII or other chaperone knocked out, respectively. Such a construct confers enhanced stability. In the Example section herein strains referred to as “Control Strain” and “Strain A” refer back to the same named strains described in WO 05/061718.

To optimize production of the heterologous proteins, it is also preferred that the host strain carries a mutation, such as the S. cerevisiae pep4 mutation (Jones, Genetics 85: 23-33, 1977), which results in reduced proteolytic activity. Host strains containing mutations in other protease encoding regions are particularly useful to produce large quantities of the Tf fusion proteins of the invention.

Host cells containing DNA constructs of the present invention are grown in an appropriate growth medium. As used herein, the term “appropriate growth medium” means a medium containing nutrients required for the growth of cells. Nutrients required for cell growth may include a carbon source, a nitrogen source, essential amino acids, vitamins, minerals and growth factors. The growth medium win generally select for cells containing the DNA construct by, for example, drug selection or deficiency in an essential nutrient which is complemented by the selectable marker on the DNA construct or co-transfected with the DNA construct. Yeast cells, for example, are preferably grown in a chemically defined medium, comprising a carbon source, e.g. sucrose, a non-amino acid nitrogen source, inorganic salts, vitamins and essential amino acid supplements. The pH of the medium is preferably maintained at a pH greater than 2 and less than 8, preferably at pH 5.5-6.5. Methods for maintaining a stable pH include buffering and constant pH control. Preferred buffering agents include succinic acid and Bis-Tris (Sigma Chemical Co., St. Louis, Mo.). Yeast cells having a defect in a gene required for asparagine-linked glycosylation are preferably grown in a medium containing an osmotic stabilizer. A preferred osmotic stabilizer is sorbitol supplemented into the medium at a concentration between 0.1 M and 1.5 M., preferably at 0.5 M or 1.0 M.

Cultured mammalian cells are generally grown in commercially available serum-containing or serum-free media. Selection of a medium appropriate for the particular cell line used is within the level of ordinary skin in the art. Transfected mammalian cells are allowed to grow for a period of time, typically 1-2 days, to begin expressing the DNA sequence(s) of interest. Drug selection is then applied to select for growth of cells that are expressing the selectable marker in a stable fashion. For cells that have been transfected with an amplifiable selectable marker the drug concentration may be increased in a stepwise manner to select for increased copy number of the cloned sequences, thereby increasing expression levels.

Baculovirus/insect cell expression systems may also be used to produce the modified Tf fusion proteins of the invention. The BacPAK™ Baculovirus Expression System (BD Biosciences (Clontech)) expresses recombinant proteins at high levels in insect host cells. The target gene is inserted into a transfer vector, which is cotransfected into insect host cells with the linearized BacPAK6 viral DNA. The BacPAK6 DNA is missing an essential portion of the baculovirus genome. When the DNA recombines with the vector, the essential element is restored and the target gene is transferred to the baculovirus genome. Following recombination, a few viral plaques are picked and purified, and the recombinant phenotype is verified. The newly isolated recombinant virus can then be amplified and used to infect insect cell cultures to produce large amounts of the desired protein.

Tf fusion proteins of the present invention may also be produced using transgenic plants and animals. For example, sheep and goats can make the therapeutic protein in their milk. Or tobacco plants can include the protein in their leaves. Both transgenic plant and animal production of proteins comprises adding a new gene coding the fusion protein into the genome of the organism. Not only can the transgenic organism produce a new protein, but it can also pass this ability onto its offspring.

Secretory Signal Sequences

The terms “secretory signal sequences” or “signal sequence” or “secretion leader sequence” are used interchangeably and are described, for example in U.S. Pat. No. 6,291,212 and U.S. Pat. No. 5,547,871, both of which are herein incorporated by reference in their entirety. Secretory signal sequences or signal sequences or secretion leader sequences encode secretory peptides. A secretory peptide is an amino acid sequence that acts to direct the secretion of a mature polypeptide or protein from a cell. Secretory peptides are generally characterized by a core of hydrophobic amino acids and are typically (but not exclusively) found at the amino termini of newly synthesized proteins. Very often the secretory peptide is cleaved from the mature protein during secretion. Secretory peptides may contain processing sites that allow cleavage of the signal peptide from the mature protein as it passes through the secretory pathway. Processing sites may be encoded within the signal peptide or may be added to the signal peptide by, for example, in vitro mutagenesis.

Secretory peptides may be used to direct the secretion of GLP-1/mTf and GLP-1/linker/GLP-1 fusion proteins of the invention. One such secretory peptide that may be used in combination with other secretory peptides is the alpha mating factor leader sequence. Secretory signal sequences or signal sequences or secretion leader sequences are required for a complex series of post-translational processing steps which result in secretion of a protein. If an intact signal sequence is present, the protein being expressed enters the lumen of the rough endoplasmic reticulum and is then transported through the Golgi apparatus to secretory vesicles and is finally transported out of the cell. Generally, the signal sequence immediately follows the initiation codon and encodes a signal peptide at the amino-terminal end of the protein to be secreted. In most cases, the signal sequence is cleaved off by a specific protease, called a signal peptidase. Preferred signal sequences improve the processing and export efficiency of recombinant protein expression using viral, mammalian or yeast expression vectors.

In one embodiment, the native Tf signal sequence may be used to express and secrete fusion proteins of the present invention. Since transferrin molecules exist in various types of secretions such as blood, tears, and milk, there are many different transferrin signal peptides. For example, the transferrin signal peptide could be from serum transferrin, lactotransferrin, or melanotransferrin. The native transferrin signal peptide also could be from various species such as insects, mammals, fish, frog, duck, chicken, or other species. Preferably, the signal peptide is from a mammalian transferrin molecule. More preferably, the signal peptide is from human serum transferrin. The table below summarizes the signal peptide sequences from various mammalian transferrin molecules (www.chatham.edu/undergraduate/bio/lambert/transferrin/signal.htm).

Signal Peptide Sequences (from GenBank entries)

Location in
immature
protein,

| | | | |
|---|---|---|---|
| Species | Type | Sequence | SEQ ID NO: |

| | | | |
|---|---|---|---|
| Mammals | | | |
| Bos taurus | serum | MRPAVRALLA | 1-19, |
| (cow) | | CAVLGLCLA | SEQ ID NO: 97 |
| Equus caballus | serum | MRLAIRALLA | 1-19, |
| (horse) | | CAVLGLCLA | SEQ ID NO: 98 |
| Homo sapiens | serum | MRLAVGALLV | amino acids |
| (human) | | CAVLGLCLA | 1-19 of |
| | | | SEQ ID NO: 2 |
| Mus musculus | serum | MRLTVGALLA | 1-19, |
| (mouse) | | CAALGLCLA | SEQ ID NO: 99 |
| Oryctolagus cuniculus | serum | MRLAAGALLA | 1-19, |
| (rabbit) | | CAALGLCLA | SEQ ID NO: 100 |
| Rattus norvegicus | serum | MRFAVGALLA | 1-19, |
| (rat) | | CAALGLCLA | SEQ ID NO: 101 |
| Sus scrofa | serum | missing in | NA* |
| (pig) | | sequence? | |
| Bos taurus | lacto | MKLFVPALLS | 1-19, |
| (cow) | | LGALGLCLA | SEQ ID NO: 102 |
| Bubalus bubalis | lacto | MKLFVPALLS | 1-19, |
| (buffalo) | | LGALGLCLA | SEQ ID NO: 103 |
| Camelus dromedaries | lacto | MKLFFPALLS | 1-19, |
| (camel) | | LGALGLCLA | SEQ ID NO: 104 |
| Capra hircus | lacto | MKLFVPALLS | 1-19, |
| (goat) | | LGALGLCLA | SEQ ID NO: 105 |
| Equus caballus | lacto | LGLCLA | 1-6, |
| (horse) | | (rest missing?) | SEQ ID NO: 106 |
| Mus musculus | lacto | MRLLIPSLIF | 1-19, |
| (mouse) | | LEALGLCLA | SEQ ID NO: 107 |
| Sus scrofa | lacto | MKLFIPALLF | 1-19, |
| (pig) | | LGTLGLCLA | SEQ ID NO: 108 |
| Sus scrofa | ica | MRLAFCVLLC | 1-19, |
| (pig) | | AGSLGLCLA | SEQ ID NO: 109 |
| Homo sapiens | melano | MRGPSGALWL | 1-19, |
| (human) | | LLALRTVLG | SEQ ID NO: 110 |
| Mus musculus | melano | MRLLSVTFWL | 1-19, |
| (mouse) | | LLSLRTVVC | SEQ ID NO: 111 |
| Oryctolagus cuniculus | melano | MRCRSAAMWI | NA* |
| (rabbit) | | FLALRTALG | 1-19, |
| | | (by inference) | SEQ ID NO: 112 |

*NA: Not available in GenBank description; sequence shown was inferred from relatives and multiple sequence alignment and checked against SignalP.
“missing in sequence”: signal peptide not included in published sequence data.

In another embodiment, the signal peptides are from variant or modified transferrin molecules that have functionally active signal peptides. Additionally, the signal peptides are variant or modified forms of transferrin signal peptides that retain the ability to transport a transferrin fusion protein of the present invention across the cell membrane and then to process the fusion protein.

In another embodiment, the transferring derived signal sequence may be used to secrete a heterologous protein, for instance, any protein of interest that is heterologous to the Tf signal sequence may be expressed and secreted using a Tf signal. In particular, a Tf signal sequence may be used to secrete proteins from recombinant yeast. Preferably, the signal peptide is from human serum transferrin (nL, amino acids 1-19 of SEQ ID NO: 2).

In order to ensure efficient removal of the signal sequence, in some cases it may be preferable to include a short pro-peptide sequence between the signal sequence and the mature protein in which the C-terminal portion of the pro-peptide comprises a recognition site for a protease, such as the yeast kex2p protease. Preferably, the pro-peptide sequence is about 2-12 amino acids in length, more preferably about 4-8 amino acids in length. Examples of such pro-peptides are Arg-Ser-Leu-Asp-Lys-Arg (SEQ ID NO: 113, Arg-Ser-Leu-Asp-Arg-Arg (SEQ ID NO: 114), Arg-Ser-Leu-Glu-Lys-Arg (SEQ ID NO: 115), and Arg-Ser-Leu-Glu-Arg-Arg (SEQ ID NO: 116).

Linkers

The Tf moiety and the therapeutic protein of the modified transferrin fusion proteins of the invention use a linker peptide of various lengths to provide greater physical separation and allow more spatial mobility between the fused proteins and thus maximize the accessibility of the therapeutic protein, for instance, for binding to its cognate receptor. In one embodiment, as discussed above, GLP-2 or a fragment thereof may be used as a linker, preferably when GLP-1 is the therapeutic protein moiety. The linker can be less than about 50, 40, 30, 20, 10, or 5 amino acid residues. The linker can be covalently linked to and between the transferrin protein or portion thereof and the therapeutic protein, such as GLP-1. These linkers may be used to link GLP-1 to transferrin.

In the preferred embodiment of the invention, GLP-1 is linked to mTf via a substantially non-helical linker. Examples of such rigid linkers include PE, PEA, PEAPTD (SEQ ID NO.: 13), (PEAPTD)₂ (SEQ ID NO.: 10), (PEAPTD)₃ (SEQ ID NO.: 14), or (PEAPTD)ₙ wherein n is an integer. The present invention also provides the IgG hinge linker, the CEx linker (SSGAPPPS; SEQ ID NO.: 15 (C-terminal extension to Exendin-4)), the IgG hinge linker in conjunction with the PEAPTD linker and the IgG hinge linker in conjunction with the CEx linker.

Detection of GLP-1/Tf Fusion Proteins

Assays for detection of biologically active modified transferrin-fusion protein may include Western transfer, protein blot or colony filter as well as activity based assays that detect the fusion protein comprising transferrin and therapeutic protein. A Western transfer filter may be prepared using the method described by Towbin et al. (Proc. Natl. Acad. Sci. USA 76: 4350-4354, 1979). Briefly, samples are electrophoresed in a sodium dodecylsulfate polyacrylamide gel. The proteins in the gel are electrophoretically transferred to nitrocellulose paper. Protein blot filters may be prepared by filtering supernatant samples or concentrates through nitrocellulose filters using, for example, a Minifold (Schleicher & Schuell, Keene, N. H.). Colony filters may be prepared by growing colonies on a nitrocellulose filter that has been laid across an appropriate growth medium. In this method, a solid medium is preferred. The cells are allowed to grow on the filters for at least 12 hours. The cells are removed from the filters by washing with an appropriate buffer that does not remove the proteins bound to the filters. A preferred buffer comprises 25 mM Tris-base, 19 mM glycine, pH 8.3, 20% methanol.

Fusion proteins of the present invention may be labeled with a radioisotope or other imaging agent and used for in in vivo diagnostic purposes. Preferred radioisotope imaging agents include iodine-125 and technetium-99, with technetium-99 being particularly preferred. Methods for producing protein-isotope conjugates are well known in the art, and are described by, for example, Eckelnan et al. (U.S. Pat. No. 4,652,440), Parker et al. (WO 87/05030) and Wilber et al. (EP 203,764). Alternatively, the transferrin fusion proteins may be bound to spin label enhancers and used for magnetic resonance (MR) imaging. Suitable spin label enhancers include stable, sterically hindered, free radical compounds such as nitroxides. Methods for labeling ligands for MR imaging are disclosed by, for example, Coffman et al. (U.S. Pat. No. 4,656,026).

Detection of a fusion protein of the present invention can be facilitated by coupling (i.e., physically linking) the therapeutic protein to a detectable substance. Examples of detectable substances include various enzymes, prosthetic groups., fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin: examples of suitable fluorescent materials include umbelliferoine, fluorescein, fluorescein isothiocyanate, rhodamine. dichlorotriazinylamine fluorescein, dansyl chloride or phycoerythrin: an example of a luminescent material includes luminol: examples of bioluminescent materials include luciferase, luciferin, and aequorin, and examples of suitable radioactive material include ¹²⁵I, ¹³¹I, ³⁵S or ³H.

In one embodiment where one is assaying for the ability of a transferrin fusion protein of the invention to bind or compete with an antigen for binding to an antibody, various immunoassays known in the art can be used, including but not limited to, competitive and non-competitive assay systems using techniques such as radioimmunoassays, ELISA (enzyme linked immunosorbent assay), sandwich immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays), complement fixation assays, immunofluorescence assays, protein A assays, and immunoelectrophoresis assays, etc. In one embodiment, the binding of the transferrin fusion protein is detected by detecting a label on the transferrin fusion protein. In another embodiment, the transferrin fusion protein is detected by detecting binding of a secondary antibody or reagent that interacts with the transferrin fusion protein. In a further embodiment, the secondary antibody or reagent is labeled. Many means are known in the art for detecting binding in an immunoassay and are within the scope of the present invention.

Fusion proteins of the invention may also be detected by assaying for the activity of the therapeutic protein moiety. Specifically, transferrin fusion proteins of the invention may be assayed for functional activity (e.g., biological activity or therapeutic activity) using assays known to one of ordinary skin in the art. Additionally, one of skin in the art may routinely assay fragments of a therapeutic protein corresponding to a therapeutic protein portion of a fusion protein of the invention, for activity using well-known assays. Further, one of skin in the art may routinely assay fragments of a modified transferrin protein for activity using assays known in the art.

For example, in one embodiment where one is assaying for the ability of a transferrin fusion protein of the invention to bind or compete with a therapeutic protein for binding to an anti-therapeutic polypeptide antibody and/or anti-transferrin antibody, various immunoassays known in the art can be used, including but not limited to, competitive and non-competitive assay systems using techniques such as radioimmunoassays, ELISA (enzyme linked immunosorbent assay), sandwich immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In a further embodiment, where a binding partner (e.g., a receptor or a ligand) of a therapeutic protein is identified, binding to that binding partner by a transferrin fusion protein containing that therapeutic protein as the therapeutic protein portion of the fusion can be assayed, e.g., by means well-known in the art, such as, for example, reducing and non-reducing gel chromatography, protein affinity chromatography, and affinity blotting. Other methods win be known to the skilled artisan and are within the scope of the invention.

Production of Fusion Proteins

The present invention further provides methods for producing a modified fusion protein of the invention using nucleic acid molecules herein described. In general terms, the production of a recombinant form of a protein typically involves the following steps.

A nucleic acid molecule is first obtained that encodes a transferrin fusion protein of the invention. The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be accomplished in a variety of ways. For example, the construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier and are otherwise known to persons skilled in the art. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce a desired recombinant protein.

As discussed above, any expression system may be used, including yeast, bacterial, animal, plant, eukaryotic and prokaryotic systems. In some embodiments, yeast, mammalian cell culture and transgenic animal or plant production systems are preferred. In other embodiments, yeast systems that have been modified to reduce native yeast glycosylation, hyper-glycosylation or proteolytic activity may be used.

Isolation/Purification of Modified Transferrin Fusion Proteins

Secreted, biologically active, modified transferrin fusion proteins may be isolated from the medium of host cells grown under conditions that allow the secretion of the biologically active fusion proteins. The cell material is removed from the culture medium, and the biologically active fusion proteins are isolated using isolation techniques known in the art. Suitable isolation techniques include precipitation and fractionation by a variety of chromatographic methods, including gel filtration, ion exchange chromatography and affinity chromatography.

A particularly preferred purification method is affinity chromatography on an iron binding or metal chelating column or an immunoaffinity chromatography using an antigen directed against the transferrin or therapeutic protein of the polypeptide fusion. The antigen is preferably immobilized or attached to a solid support or substrate. A particularly preferred substrate is CNBr-activated Sepharose (Pharmacia LKB Technologies. Inc., Piscataway. N.J.). By this method., the medium is combined with the antigen/substrate under conditions that win allow binding to occur. The complex may be washed to remove unbound material. and the transferrin fusion protein is released or eluted through the use of conditions unfavorable to complex formation. Particularly useful methods of elution include changes in pH, wherein the immobilized antigen has a high affinity for the transferrin fusion protein at a first pH and a reduced affinity at a second (higher or lower) pH; changes in concentration of certain chaotropic agents: or through the use of detergents.

Delivery of a Drug or Therapeutic Protein to the Inside of a Cell and/or Across the Blood Brain Barrier (BBB)

Within the scope of the invention, the modified transferrin fusion proteins may be used as a carrier to deliver a molecule or small molecule therapeutic complexed to the ferric ion of transferrin to the inside of a cell or across the blood brain barrier or other barriers including across the cell membrane of any cell type that naturally or engineered to express a Tf receptor. In these embodiments, the Tf fusion protein win typically be engineered or modified to inhibit, prevent or remove glycosylation to extend the serum half-life of the fusion protein and/or therapeutic protein portion. The addition of a targeting peptide is specifically contemplated to further target the Tf fusion protein to a particular cell type, e.g., a cancer cell.

In one embodiment, the iron-containing, anti-anemic drug, ferric-sorbitol-citrate complex is loaded onto a modified Tf fusion protein of the invention. Ferric-sorbitol-citrate (FSC) has been shown to inhibit proliferation of various murine cancer cells in vitro and cause tumor regression in vivo, while not having any effect on proliferation of non-malignant cells (Poljak-Blazi et al. (June 2000) Cancer Biotherapy and Radiopharmaceuticals (United States), 15/3:285-293).

In another embodiment, the antineoplastic drug Adriamycin® (doxorubicin) and/or the chemotherapeutic drug bleomycin, both of which are known to form complexes with ferric ion, is loaded onto a Tf fusion protein of the invention. In other embodiments, a salt of a drug, for instance, a citrate or carbonate salt, may be prepared and complexed with the ferric iron that is then bound to Tf. As tumor cells often display a higher turnover rate for iron; transferrin modified to carry at least one anti-tumor agent, may provide a means of increasing agent exposure or load to the tumor cells. (Demant, E. J., (1983) Eur. J. Biochem. 137/(1-2): 113-118, Padbury et al. (1985) J. Biol. Chem. 260/13:7820-7823).

Pharmaceutical Formulations and Treatment Methods

In one aspect of the present invention, the pharmaceutical compositions comprising the GLP-1/linker/mTf proteins may be formulated by any of the established methods of formulating pharmaceutical compositions, e.g. as described in Remington's Pharmaceutical Sciences, 1985. In one embodiment, the pharmaceutical composition comprises a fusion protein is SEQ ID NO.: 12. The composition may be in a form suited for systemic injection or infusion and may, as such, be formulated with a suitable liquid vehicle such as sterile water or an isotonic saline or glucose solution. The compositions may be sterilized by conventional sterilization techniques which are well known in the art. The resulting aqueous solutions may be packaged for use or filtered under aseptic conditions and lyophilized, the lyophilized preparation being combined with the sterile aqueous solution prior to administration. The composition may contain pharmaceutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

The GLP-1/linker/mTf fusion proteins of the present invention may also be adapted for oral, nasal, transdermal, pulmonal or rectal administration (see PCT/US03/26778, which is herein incorporated by reference in its entirety). The pharmaceutically acceptable carrier or diluent employed in the composition may be any conventional solid carrier. Examples of solid carriers are lactose, terra alba, sucrose, talc, gelatin, agar, pectin, acacia, magnesium stearate and stearic acid. Similarly, the carrier or diluent may include any sustained release material known in the art, such as glyceryl monostearate or glyceryl distearate, alone or mixed with a wax.

It may be of particular advantage to provide the composition of the invention in the form of a sustained release formulation. As such, the composition may be formulated as microcapsules or microparticles containing the GLP-1/linker/mTf encapsulated by or dispersed in a suitable pharmaceutically acceptable biodegradable polymer such as polylactic acid, polyglycolic acid or a lactic acid/glycolic acid copolymer.

For nasal administration, the preparation may contain GLP-1/linker/mTf dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

Generally, the compounds of the present invention are dispensed in unit dosage form comprising 0.5-500 mg of the fusion protein together with a pharmaceutically acceptable carrier per unit dosage.

Moreover, the present invention contemplates the use of the GLP-1/linker/mTf for the manufacture of a medicinal product which can be used in the treatment of diseases associated with elevated glucose level, such as but not to limited to those described above. Specifically, the present invention contemplates the use of GLP-1/transferrin fusion protein for the treatment of diabetes including type II diabetes, obesity, severe bums, and heart failure, including congestive heart failure and acute coronary syndrome.

The N-terminus of GLP-1 is normally amidated. In yeast, amidation does not occur. In one aspect of the invention, in order to compensate for amidation on the N-terminus which does not occur in yeast, an extra amino acid is added on the N-terminus of GLP-1. The addition of an amino acid to the N-terminus of GLP-1 may prevent dipeptidyl peptidase from cleaving at the second amino acid of GLP-1 due to steric hindrance. Therefore, GLP-1 win remain functionally active. Any one of the 20 amino acids may be added to the N-terminus of GLP-1. In some instances, an uncharged or positively charged amino acid maybe used and preferably, Histidine is added. The GLP-1 with the extra amino acid is then fused to transferrin. Accordingly, the GLP-1 with the added amino acid will be fused at the N-terminus of the transferrin moiety, leaving a free GLP-1 N-terminal end.

In one embodiment of making the GLP-1(7-36) or GLP-1(7-37) peptide more resistant to cleavage by dipeptidyl peptidase, a His residue is added at the N-terminus of GLP-1 or is inserted after the His residue at the N-terminus of GLP-1, so that the N-terminus of GLP-1 begins with His-His.

In another embodiment of the invention, the second residue from the N-terminus in the GLP-1(7-36) or GLP-1(7-37) peptide (SEQ ID NO: 6) is substituted with another amino acid. For example, the Ala residue at the second residue from the N-terminus in the GLP-1(7-36) or GLP-1(7-37) peptide may be substituted with Ser, Gly, Val, or another amino acid.

The GLP-1/linker/mTf fusion proteins of the invention may be administered to a patient in need thereof using standard administration protocols. For instance, the fusion proteins of the present invention can be provided alone, or in combination, or in sequential combination with other agents that modulate a particular pathological process. As used herein, two agents are said to be administered in combination when the two agents are administered simultaneously or are administered independently in a fashion such that the agents win act at the same or near the same time.

The fusion proteins of the present invention can be administered via parenteral, subcutaneous, intravenous, intramuscular, intraperitoneal, transdermal and buccal routes. For example, an agent may be administered locally to a site of injury via microinfusion. Alternatively, or concurrently, administration may be noninvasive by either the oral, inhalation, nasal, or pulmonary route. The dosage administered win be dependent upon the age, health, and weight of the recipient, kind of concurrent treatment, if any, frequency of treatment, and the nature of the effect desired.

While any method of administration may be used to deliver the fusion proteins of the invention, administration or delivery orally may be a preferred embodiment for certain classes of fusion proteins or to treat certain conditions.

The present invention further provides compositions containing one or more fusion proteins of the invention. While individual needs vary, determination of optimal ranges of effective amounts of each component is within the skin of the art. Typical dosages comprise about 1 pg/kg to about 150 mg/kg body weight. In one embodiment, dosages for systemic administration comprise about 100 ng/kg to about 100 mg/kg body weight. In another embodiment, doses range from 300 μg/kg to 900 μg/kg. The present invention also includes dosing weekly at a total dose of about 50 mg, 100 mg, or 150 mg. Other dosages for direct administration to a site via microinfusion comprise about 1 ng/kg to about 1 mg/kg body weight. When administered via direct injection or microinfusion, modified fusion proteins of the invention may be engineered to exhibit reduced or no binding of iron to prevent, in part, localized iron toxicity.

In addition to the pharmacologically active fusion protein, the compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example. sesame oil, or synthetic fatty acid esters, for example, ethyl oleate or triglycerides. Aqueous injection suspensions may contain substances which increase the viscosity of the suspension and include, for example, sodium carboxymethyl cellulose, sorbitol and dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient. Suitable formulations for oral administration include hard or soft gelatin capsules, pins, tablets, including coated tablets, elixirs, suspensions, syrups or inhalations and controlled release forms thereof.

The pharmaceutical composition of the present invention can be in unit dosage form, e.g. as tablets or capsules. In such form the composition is sub-divided in unit dose containing appropriate quantities of the active ingredient; the unit dosage forms can be packaged compositions, for example, packeted powders, vials, ampoules, prefined syringes or sachets containing liquids. The unit dosage form can be, for example, a capsule or tablet itself, or it can be the appropriate number of any such compositions in package form. The dosage to be used in the treatment must be subjectively determined by the physician.

In practicing the methods of this invention, the fusion proteins of this invention may be used alone or in combination, or in combination with other therapeutic or diagnostic agents. In certain preferred embodiments, the compounds of this invention may be co-administered along with other compounds typically prescribed for these conditions according to generally accepted medical practice. The compounds of this invention can be utilized in vivo, ordinarily in mammals, such as humans, sheep, horses, cattle, pigs, dogs, cats, rats and mice, or in vitro.

In the present invention, fusion proteins, including but not limited to modified Tf fusion proteins, may be formulated for oral delivery. In particular. certain fusion proteins of the invention that are used to treat certain classes of diseases or medical conditions may be particularly amenable for oral formulation and delivery. Such classes of diseases or conditions include, but are not limited to, acute, chronic and recurrent diseases. Chronic or recurrent diseases include, but are not limited to, viral disease or infections, cancer, a metabolic diseases, obesity, autoimmune diseases, inflammatory diseases, allergy, graft-vs.-host disease, systemic microbial infection, anemia, cardiovascular disease, psychosis. genetic diseases, neurodegenerative diseases, disorders of hematopoietic cells, diseases of the endocrine system or reproductive systems, gastrointestinal diseases. Examples of these classes of disease include diabetes, multiple sclerosis, asthma, HCV or HIV infections, hypertension, hypercholesterolemia, arterial scherosis, arthritis, and Alzheimer's disease. In many chronic diseases, oral formulations of Tf fusion proteins of the invention and methods of administration are particularly useful because they allow long-term patient care and therapy via home oral administration without reliance on injectable treatment or drug protocols.

Oral formulations and delivery methods comprising fusion proteins of the invention take advantage of, in part, transferrin receptor mediated transcytosis across the gastrointestinal (GI) epithelium. The Tf receptor is found at a very high density in the human GI epithelium, transferrin is highly resistant to tryptic and chymotryptic digestion and Tf chemical conjugates have been used to successfully deliver proteins and peptides across the GI epithelium (Xia et al., (2000) J. Pharmacol. Experiment. Therap., 295:594-600; Xia et al. (2001) Pharmaceutical Res., 18(2): 191-195; and Shah et al. (1996) J. Pharmaceutical Sci., 85(12):1306-1311, all of which are herein incorporated by reference in their entirety). Once transported across the GI epithelium, fusion proteins of the invention exhibit extended half-life in serum, that is, the therapeutic protein or peptide(s) attached or inserted into Tf exhibit an extended serum half-life compared to the protein or peptide in its non-fused state.

Oral formulations of fusion proteins of the invention may be prepared so that they are suitable for transport to the GI epithelium and protection of the fusion protein component and other active components in the stomach. Such formulations may include carrier and dispersant components and may be in any suitable form, including aerosols (for oral or pulmonary delivery), syrups, elixirs, tablets, including chewable tablets, hard or soft capsules, troches, lozenges, aqueous or oily suspensions, emulsions, cachets or pellets granulates, and dispersible powders. Preferably, fusion protein formulations are employed in solid dosage forms suitable for simple, and preferably oral, administration of precise dosages. Solid dosage forms for oral administration are preferably tablets, capsules, or the like.

For oral administration in the form of a tablet or capsule, care should be taken to ensure that the composition enables sufficient active ingredient to be absorbed by the host to produce an effective response. Thus, for example, the amount of fusion protein may be increased over that theoretically required or other known measures such as coating or encapsulation may be taken to protect the polypeptides from enzymatic action in the stomach.

Traditionally, peptide and protein drugs have been administered by injection because of the poor bioavailability when administered orally. These drugs are prone to chemical and conformational instability and are often degraded by the acidic conditions in the stomach, as well as by enzymes in the stomach and gastrointestinal tract. In response to these delivery problems, certain technologies for oral delivery have been developed, such as encapsulation in nanoparticles composed of polymers with a hydrophobic backbone and hydrophilic branches as drug carriers, encapsulation in microparticles, insertion into liposomes in emulsions, and conjugation to other molecules. All of which may be used with the fusion molecules of the present invention.

Examples of nanoparticles include mucoadhesive nanoparticles coated with chitosan and Carbopol (Takeuchi et al., Adv. Drug Deliv. Rev. 47(1):39-54, 2001) and nanoparticles containing charged combination polyesters, poly(2-sulfobutyl-vinyl alcohol) and poly(D,L-lactic-co-glycolic acid) (Jung et al., Eur. J. Pharm. Biopharm. 50(1):147-160, 2000). Nanoparticles containing surface polymers with poly-N-isopropylacrylamide regions and cationic poly-vinylamine groups showed improved absorption of salmon calcitonin when administered orally to rats.

Drug delivery particles composed of alginate and pectin, strengthened with polylysine, are relatively acid and base resistant and can be used as a carrier for drugs. These particles combine the advantages of bioadhesion, enhanced absorption and sustained release (Liu et al., J. Pharm. Pharmacol. 51(2):141-149, 1999).

Additionally, lipoamino acid groups and liposaccharide groups conjugated to the N- and C-termini of peptides such as synthetic somatostatin, creating an amplipathic surfactant, were shown to produce a composition that retained biological activity (Toth et al., J. Med. Chem. 42(19):4010-4013, 1999).

Examples of other peptide delivery technologies include carbopol-coated mucoadhesive emulsions containing the peptide of interest and either nitroso-N-acetyl-D,L-penicillamine and carbolpol or taurochlolate and carbopol. These were shown to be effective when orally administered to rats to reduce serum calcium concentrations (Ogiso et al., Biol. Pharm. Bull. 24(6):656-661, 2001). Phosphatidylethanol, derived from phosphatidylcholine, was used to prepare liposomes containing phosphatidylethanol as a carrier of insulin. These liposomes, when administered orally to rats, were shown to be active (Kisel et al., Int. J. Pharm. 216(1-2):105-114, 2001).

Insulin has also been formulated in poly(vinyl alcohol)-gel spheres containing insulin and a protease inhibitor, such as aprotinin or bacitracin. The glucose-lowering properties of these gel spheres have been demonstrated in rats, where insulin is released largely in the lower intestine (Kimura et al., Biol. Pharm. Bull. 19(6):897-900, 1996.

Oral delivery of insulin has also been studied using nanoparticles made of poly(alkyl cyanoacrylate) that were dispersed with a surfactant in an oily phase (Damge et al., J. Pharm. Sci. 86(12):1403-1409, 1997) and using calcium alginate beads coated with chitosan (Onal et al., Artif. Cells Blood Substit. Immobil. Biotechnol. 30(3):229-237, 2002).

In other methods, the N- and C-termini of a peptide are linked to polyethylene glycol and then to allyl chains to form conjugates with improved resistance to enzymatic degradation and improved diffusion through the GI wall (www.nobexcorp.com).

BioPORTER® is a cationic lipid mixture, which interacts non-covalently with peptides to create a protective coating or layer. The peptide-lipid complex can fuse to the plasma membrane of cells, and the peptides are internalized into the cells (www.genetherapysystems.com).

In a process using liposomes as a starting material, cochleate-shaped particles have been developed as a pharmaceutical vehicle. A peptide is added to a suspension of liposomes containing mainly negatively charged lipids. The addition of calcium causes the collapse and fusion of the liposomes into large sheets composed of lipid bilayers, which then spontaneously roll up or stack into cochleates (U.S. Pat. No. 5,840,707; www.biodeliverysciences.com).

Compositions comprising fusion protein intended for oral use may be prepared according to any method known to the alp for the manufacture of pharmaceutical compositions and such compositions may contain one or more agents selected from the group consisting of sweetening agents in order to provide a pharmaceutically elegant and palatable preparation. For example, to prepare orally deliverable tablets, Tf fusion protein is mixed with at least one pharmaceutical excipient, and the solid formulation is compressed to form a tablet according to known methods, for delivery to the gastrointestinal tract. The tablet composition is typically formulated with additives, e.g., a saccharide or cellulose carrier, a binder such as starch paste or methyl cellulose, a finer, a disintegrator, or other additives typically usually used in the manufacture of medical preparations. To prepare orally deliverable capsules, DHEA is mixed with at least one pharmaceutical excipient, and the solid formulation is placed in a capsular container suitable for delivery to the gastrointestinal tract. Compositions comprising fusion protein may be prepared as described generally in Remington's Pharmaceutical Sciences, 18th Ed. 1990 (Mack Publishing Co. Easton Pa. 18042) at Chapter 89, which is herein incorporated by reference.

As described above, many of the oral formulations of the invention may contain inert ingredients which allow for protection against the stomach environment, and release of the biologically active material in the intestine. Such formulations, or enteric coatings, are well known in the art. For example, tablets containing Tf fusion protein in admixture with non-toxic pharmaceutically acceptable excipients which are suitable for manufacture of tablets may be used. These excipients may be inert diluents, such as calcium carbonate, sodium carbonate, lactose, calcium phosphate or sodium phosphate; granulating and disintegrating agents, for example, maize starch, gelatin or acacia, and lubricating agents, for example, magnesium stearate, stearic acid, or talc.

The tablets may be uncoated or they may be coated with known techniques to delay disintegration and absorption in the gastrointestinal track and thereby provide a sustained action over a longer period of time. For example, a time delay material such as glyceryl monostearate or glyceryl distearate alone or with a wax may be employed.

Formulations for oral use may also be presented as hard gelatin capsules wherein the active ingredient is mixed with an inert solid diluent, for example, calcium carbonate, calcium phosphate, or kaolin or as soft gelatin capsules wherein the active ingredient is mixed with an aqueous or an oil medium, for example, arachis oil, peanut oil, liquid paraffin or olive oil.

Aqueous suspensions may contain fusion protein in the admixture with excipients suitable for the manufacture of aqueous suspensions. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents may be a naturally occurring phosphatide, for example, lecithin, or condensation products of an alkylene oxide with fatty acids, for example, polyoxyethylene stearate, or condensation products of ethylene oxide with long chain aliphatic alcohols, for example, heptadecylethyloxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate. The aqueous suspensions may also contain one or more preservatives for example, ethyl or n-propyl p-hydroxybenzoate, one or more coloring agents, one or more flavoring agents and one or more sweetening agents such as sucrose or saccharin.

Oily suspensions may be formulated by suspending the active ingredient in a vegetable oil, for example, arachis oil, olive oil, sesame oil or coconut oil, or in a mineral oil such as liquid paraffin. The oil suspensions may contain a thickening agent, for example, beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient and admixture with dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The pharmaceutical compositions containing fusion protein may also be in the form of oil-in-water emulsions. The oil phase may be a vegetable oil, for example. olive oil or arachis oil, or a mineral oil for example, gum acacia or gum tragacanth, naturally-occurring phosphotides, for example soybean lecithins and esters or partial esters derived from fatty acids and hexitol anhydrides, for example, sorbitan monooleate, and condensation products of the same partial esters with ethylene oxide, for example, polyoxyethylene sorbitan monooleate. The emulsions may also contain sweetening and flavoring agents.

Syrups and elixirs containing fusion protein may be formulated with sweetening agents, for example, glycerol, sorbitol or sucrose. Such formulations may also contain a demulcent, a preservative and flavoring and coloring agents. The pharmaceutical compositions may be in the form of a sterile injectable preparation, for example, as a sterile injectable aqueous or oleaginous suspension. This suspension may be formulated according to the known art using those suitable dispersing or wetting agents and suspending agents which have been mentioned above. The sterile injectable preparations may also be a sterile injectable solution or suspension in a non-toxic parenterally-acceptable diluent or solvate, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this period any bland fixed oil may be employed including synthetic mono- or diglycerides. In addition, fatty acids such as oleic acid find use in the preparation of injectables.

Pharmaceutical compositions may also be formulated for oral delivery using polyester microspheres, zein microspheres, proteinoid microspheres, polycyanoacrylate microspheres, and lipid-based systems (see, for example, DiBase and Morrel, Oral Delivery of Microencapsulated Proteins, in Protein Delivery: Physical Systems, Sanders and Hendren (eds.), pages 255-288 (Plenum Press 1997)).

The proportion of pharmaceutically active fusion protein to carrier and/or other substances may vary from about 0.5 to about 100 wt. % (weight percent). For oral use, the pharmaceutical formulation win generally contain from about 5 to about 100% by weight of the active material. For other uses, the formulation win generally have from about 0.5 to about 50 wt. % of the active material.

Fusion protein formulations employed in the invention provide an effective amount of fusion protein upon administration to an individual. As used in this context, an “effective amount” of fusion is an amount that is effective to ameliorate a symptom of a disease.

The fusion protein composition of the present invention may be, though not necessarily, administered daily, in an effective amount to ameliorate a symptom. Generally, the total daily dosage win be at least about 50 mg, preferably at least about 100 mg, and more preferably at least about 200 mg, and preferably not more than 500 mg per day, administered orally, e.g., in 4 capsules or tablets, each containing 50 mg Tf fusion protein. Capsules or tablets for oral delivery can conveniently contain up to a full daily oral dose, e.g., 200 mg or more.

In a particularly preferred embodiment, oral pharmaceutical compositions comprising fusion protein are formulated in buffered liquid form which is then encapsulated into soft or hard-coated gelatin capsules which are then coated with an appropriate enteric coating. For the oral pharmaceutical compositions of the invention, the location of release may be anywhere in the GI system, including the small intestine (the duodenum, the jejunum, or the ileum), or the large intestine.

In other embodiments, oral compositions of the invention are formulated to slowly release the active ingredients, including the fusion proteins of the invention, in the GI system using known delayed release formulations.

Tf fusion proteins of the invention for oral delivery are capable of binding the receptor found in the Gl epithelium. To facilitate this binding and receptor mediated transport, fusion proteins of the invention are typically produced with iron and in some instances carbonate, bound to the moiety. Processes and methods to load the moiety of the fusion protein compositions of the invention with iron and carbonate are known in the art

In some pharmaceutical formulations of the invention, the moiety of the fusion protein may be modified to increase the affinity or affinity of the moiety to iron. Such methods are known in the art. For instance, mutagenesis can be used to produce mutant transferrin moieties that bind iron more avidly than natural transferrin. In human serum transferrin, the amino acids which are ligands for metal ion chelation include, but are not limited to N lobe amino acids Asp63, Tyr95, Tyr 188, Lys206, His207 and His249; and C lobe amino acids Asp392, Tyr426, Tyr517 and His585 of SEQ ID NO: 3 (the number beside the amino acid indicates the position of the amino acid residue in the primary amino acid sequence where the valine of the mature protein is designated position 1). See U.S. Pat. No. 5,986,067, which is herein incorporated be reference. In one embodiment, the Lys206 and His207 residues within the N lobe are replaced with Gln and Glu, respectively.

In some pharmaceutical formulations of the invention, the fusion protein is engineered to contain a cleavage site between the therapeutic protein or peptide and the moiety. Such cleavable sites or linkers are known in the art.

Pharmaceutical compositions of the invention and methods of the invention may include the addition of a transcytosis enhancer to facilitate transfer of the fusion protein across the GI epithelium. Such enhancers are known in the art. See Xia et al., (2000) J. Pharmacol. Experiment. Therap., 295:594-600; and Xia et al. (2001) Pharmaceutical Res., 18(2):191-195.

In preferred embodiments of the invention, oral pharmaceutical formulations include fusion proteins comprising a modified moiety exhibiting reduced or no glycosylation fused at the N terminal end to a GLP-1 protein or peptide as described above. Such pharmaceutical compositions may be used to treat glucose imbalance disorders such as diabetes by oral administration of the pharmaceutical composition comprising an effective dose of fusion protein.

The effective dose of fusion protein may be measured in a numbers of ways, including dosages calculated to alleviate symptoms associated with a specific disease state in a patient, such as the symptoms of diabetes. In other formulations, dosages are calculated to comprise an effective amount of fusion protein to induce a detectable change in blood glucose levels in the patient. Such detectable changes in blood glucose may include a decrease in blood glucose levels of between about 1% and 90%, or between about 5% and about 80%. These decreases in blood glucose levels win be dependent on the disease condition being treated and pharmaceutical compositions or methods of administration may be modified to achieve the desired result for each patient. In other instances, the pharmaceutical compositions are formulated and methods of administration modified to detect an increase in the activity level of the therapeutic protein or peptide in the patient, for instance, detectable increases in the activities of insulin or GLP-1. Such formulations and methods may deliver between about 1 pg to about 150 mg /kg body weight of fusion protein, about 100 ng to about 100 μg/kg body weight of fusion protein, about 100 μg/kg to about 100 mg/kg body weight of fusion protein, about 1 μg to about 1 g of fusion protein, about 10 μg to about 100 mg of fusion protein or about 10 mg to about 50 mg of fusion protein. In one embodiment, the effective close is 300 μg/kg to 900 μg/kg. In another embodiment, the effective dose is administered weekly for a total dose of about 50 mg, 100 mg, or 150 mg.

Formulations for effective dose may also be calculated using a unit measurement of therapeutic protein activity, such as about 5 to about 500 units of human insulin or about 10 to about 100 units of human insulin. The measurements by weight or activity can be calculated using known standards for each therapeutic protein or peptide fused to Tf.

If the fusion protein of the invention is used for the treatment of diabetes, efficacy can be measured by a decline in glycated hemoglobin (HbA_{1c}), which is the measure of glycemic control in chronic dosing. For instance, the effective dose can be the dose in which there is at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 15 fold, at least about 20 fold, at least about 30 fold, at least about 40 fold, at least about 50 fold, or at least about 100 fold or more decrease in glycated hemoglobin as measured by methods known in the art.

The invention also includes methods of orally administering the pharmaceutical compositions of the invention. Such methods may include, but are not limited to, steps of orally administering the compositions by the patient or a caregiver. Such administration steps may include administration on intervals such as once or twice per day depending on the fusion protein, disease or patient condition or individual patient. Such methods also include the administration of various dosages of the individual fusion protein. For instance, the initial dosage of a pharmaceutical composition may be at a higher level to induce a desired effect, such as reduction in blood glucose levels. Subsequent dosages may then be decreased once a desired effect is achieved. These changes or modifications to administration protocols may be done by the attending physician or hearth care worker. In some instances, the changes in the administration protocol may be done by the individual patient, such as when a patient is monitoring blood glucose levels and administering a mTf-GLP-1 oral composition of the invention.

The invention also includes methods of producing oral compositions or medicant compositions of the invention comprising formulating a Tf fusion protein of the invention into an orally administerable form. In other instances, the invention includes methods of producing compositions or medicant compositions of the invention comprising formulating a Tf fusion protein of the invention into a form suitable for oral administration.

Moreover, the present invention includes pulmonary delivery of the Tf fusion protein formulations. Pulmonary delivery is particularly promising for the delivery of macromolecules which are difficult to deliver by other routes of administration. Such pulmonary delivery can be effective both for systemic delivery and for localized delivery to treat diseases of the lungs, since drugs delivered to the lung are readily absorbed through the alveolar region directly into the blood circulation.

The present invention provides compositions suitable for forming a drug dispersion for oral inhalation (pulmonary delivery) to treat various conditions or diseases. The fusion protein formulation could be delivered by different approaches such as liquid nebulizers, aerosol-based metered dose inhalers (MDI's), and dry powder dispersion devices. In formulating compositions for pulmonary delivery, pharmaceutically acceptable carriers including surface active agents or surfactants and bulk carriers are commonly added to provide stability, dispersibility, consistency, and/or bulking characteristics to enhance uniform pulmonary delivery of the composition to the subject.

Surface active agents or surfactants promote absorption of polypeptide through mucosal membrane or lining. Useful surface active agents or surfactants include fatty acids and salts thereof, bile salts, phospholipid, or an alkyl saccharide. Examples of fatty acids and salts thereof include sodium, potassium and lysine salts of caprylate (C₈), caprate (C₁₀), laurate (C₁₂) and myristate (C₁₄). Examples of bile salts include cholic acid, chenodeoxycholic acid, glycocholic acid, taurocholic acid, glycochenodeoxycholic acid, taurochenodeoxycholic acid, deoxycholic acid, glycodeoxycholic acid, taurodeoxycholic acid, lithocholic acid, and ursodeoxycholic acid.

Examples of phospholipids include single-chain phospholipids, such as lysophosphatidylcholine, lysophosphatidylglycerol, lysophosphatidyletanolamine. lysophosphatidylinositol and lysophosphatidylserine; or double-chain phospholipids, such as diacylphosphatidylcholines, diacylphosphatidylglycerols, diacyphosphatidylethanolamines, diacylphosphatidylinositols and diacylphosphatidylserines. Examples of alkyl saccharides include alkyl glucosides or alkyl maltosides, such as decyl glucoside and dodecyl maltoside.

Pharmaceutical excipients that are useful as carriers include stabilizers such as human serum albumin (HSA); bulking agents such as carbohydrates, amino acids and polypeptides; pH adjusters or buffers; salts such as sodium chloride; and the like. These carriers may be in a crystalline or amorphous form or may be a mixture of the two.

Examples of carbohydrates for use as bulking agents include monosaccharides such as galactose, D-mannose, sorbose, and the like; disaccharides, such as lactose. trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. Examples of polypeptides for use as bulking agents include aspartame. Amino acids include alanine and glycine, with glycine being preferred.

Additives, which are minor components of the composition, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate is preferred.

The Tf fusion compositions for pulmonary delivery may be packaged as unit doses where a therapeutically effective amount of the composition is present in a unit dose receptacle, such as a blister pack, gelatin capsule, or the like. The manufacture of blister packs or gelatin capsules is typically carried out by methods that are generally well known in the packaging art.

U.S. Pat. No. 6,524,557 discloses a pharmaceutical aerosol formulation comprising (a) a HFA propellant; (b) a pharmaceutically active polypeptide dispersible in the propellant; and (c) a surfactant which is a C₈-C₁₆ fatty acid or salt thereof, a bile salt, a phospholipid, or an alkyl saccharide, which surfactant enhances the systemic absorption of the polypeptide in the lower respiratory tract. The invention also provides methods of manufacturing such formulations and the use of such formulations in treating patients.

One approach for the pulmonary delivery of dry powder drugs utilizes a hand-held device with a hand pump for providing a source of pressurized gas. The pressurized gas is abruptly released through a powder dispersion device, such as a venturi nozzle, and the dispersed powder made available for patient inhalation.

Dry powder dispersion devices are described in several patents. U.S. Pat. No. 3,921,637 describes a manual pump with needles for piercing through a single capsule of powdered medicine. The use of multiple receptacle disks or strips of medication is described in European Patent Application No. EP 0 467 1 72; International Patent Publication Nos. WO 91/02558; and WO 93/09832; U.S. Pat. Nos. 4,627,432; 4,811,731; 5,035,237; 5,048,514; 4,446,862; 5,048,514, and 4,446,862.

The aerosolization of protein therapeutic agents is disclosed in European Patent Application No. EP 0 289 336. Therapeutic aerosol formulations are disclosed in International Patent Publication No. WO 90/09781.

The present invention provides formulating Tf fusion protein for oral inhalation. The formulation comprises Tf fusion protein and suitable pharmaceutical excipients for pulmonary delivery. The present invention also provides administering the Tf fusion protein composition via oral inhalation to subjects in need thereof.

GLP-1-mTf Fusion Protein for Treating Type 2 Diabetes

As discussed above, GLP-1 activates and regulates important endocrine hormone systems in the body and plays a critical management role in the metabolism of glucose. Unlike all other diabetic treatments on the market GLP-1 has the potential to be restorative by acting as a growth factor for β-cells thus improving the ability of the pancreas to secrete insulin and also to make the existing insulin levels act more efficiently by improving sensitivity and better stabilizing glucose levels. This reduces the burden on daily monitoring of glucose levels and potentially offers a delay in the serious long-term side effects caused by fluctuations in blood glucose due to diabetes. Furthermore, GLP-1 can reduce appetite and reduce weight. Obesity is an inherent consequence of poor control of glucose metabolism and this only serves to aggravate the diabetic condition.

Clinical application of natural GLP-1 is limited because it is rapidly degraded in the circulation (half-life is several minutes). To maintain therapeutic levels in the circulation requires constant administration of high doses using pumps or patch devices which adds to the cost of treatment. This is inconvenient for long term chronic use especially in conjunction with all the other medications for treating diabetes and monitoring of glucose levels. The GLP-1/linker-/mTf fusion proteins retain the activity of GLP-1 but have the long half-life, solubility, and biodistribution properties of modified transferrin (mTf). These properties could provide for a low cost, small volume, monthly s.c. (subcutaneous) injection and this type of product is absolutely needed for long term chronic use.

Preferably, the GLP-1/linker/mTf fusion protein of the present invention is used to treat diabetes or obesity. A substantially non-helical, i.e., rigid, linker may be used as the linker, including, but not limited to PEAPTD (SEQ ID NO.: 13), PEAPTDPEAPTD (SEQ ID NO.: 10), PEAPTDPEAPTDPEAPTD (SEQ ID NO.: 14), IgG hinge region (SEQ ID NO.: 88, 89, and 117), and IgG hinge region and PEAPTD (SEQ ID NOS.: 118-123 and 126-129). In one embodiment, the GLP-1/linker/mTf fusion protein is BRX0585 (SEQ ID NO.: 12) as described in Example 4 and consists of GLP-1(7-37) with A8G and K34A modifications, a PEAPTDPEAPTD linker and mTf lacking N-glycosylation at sites 413 and 611 via substitution of the adjacent S/T residues.

GLP-2 may also be used as a linker to link GLP-1 to mTf. In one embodiment, the GLP-2 peptide in the fusion protein has been modified to form a more stable fusion protein resulting in a more potent GLP-1 . For example, GLP-2 peptide could be modified to have substantially reduced GLP-2 activity. The GLP-2 peptide could be modified by deleting an amino acid corresponding to H1 of the peptide. GLP-2 peptide also could be modified to have substantially reduced protease cleavage, wherein the protease cleavage is mediated by Yap3p. For instance, the amino acid corresponding to K30 of GLP-2 could be mutated, for example to an A. Alternatively, GLP-2 could be modified by deleting the amino acids corresponding to K30 to R34 or by deleting the amino acids corresponding to H1 to D8.

In another embodiment, the fusion protein comprises at least two GLP-1 peptides at the N-terminus. Moreover, the GLP-1 could be modified, for example, by adding an amino acid to the N-terminus of GLP-1. Preferably, the added amino acid is H or G. Alternatively, GLP-1 could be modified by mutating A8 to S. GLP-1 could be modified by mutating K34 to Q, A, or N. GLP-1 also could be modified by deleting V33 to R36.

GLP-1-mTf Fusion Protein in Combination with Other Therapeutic Agents

In one aspect of the invention, the GLP-1/linker/mTf fusion protein of the present invention, such as the protein corresponding to SEQ ID NO.: 12, is used in combination with at least one second therapeutic molecule such as a DPPIV inhibitor, a neutral endopeptidase (NEP) 24.11 inhibitor or Glucophage® (metformin hydrochloride tablets) or Glucophage® XR (metformin hydrochloride extended-release tablets) to treat type II diabetes, obesity, and other diseases or conditions associated with abnormal glucose levels.

Glucophage® and Glucophage® XR are oral antihyperglycemic drugs for the management of type II diabetes. Glucophage® XR is an extended release formulation of Glucophage. Accordingly, Glucophage® XR may be taken once daily because the drug is released slowly from the dosage form. Glucophage® helps the body produce less glucose from the liver. Accordingly, Glucophage® is effective in controlling blood sugar level in a patient. Glucophage® rarely causes low blood glucose (hypoglycemia) because it does not cause the body to make more insulin.

Glucophage® also helps lower the fatty blood components, triglycerides and cholesterol, that are often high in people with Type II diabetes. Metformin has been shown to decrease the appetite and help people lose a few pounds when they starting taking the medicine.

Metformin has been approved for treatment with sulfonylureas, or with insulin, or as monotherapy (by itself). Metformin has been suggested for use in treating various cardiovascular diseases such as hypertension in insulin resistant patients (WO 9112003-Upjohn), for dissolving blood clots (in combination with a t-PA-derivative) (WO 9108763, WO 9108766, WO 9108767 and WO 9108765-Boehringer Mannheim), ischemia and tissue anoxia (EP 283369-Lipha), atherosclerosis (DE 1936274-Brunnengraber & Co., DE 2357875-Hurka, and U.S. Pat. No. 4,205,087-ICI). In addition, it has been suggested to use metformin in combination with prostaglandin-analogous cyclopentane derivatives as coronary dilators and for blood pressure lowering (U.S. Pat. No. 4,182,772-Hoechst). Metformin has also been suggested for use in cholesterol lowering when used in combination with 2-hydroxy-3,3,3-trifluoropropionic acid derivatives (U.S. Pat. No. 4,107,329-ICI), 1,2-diarylethylene derivatives (U.S. Pat. No. 4,061,772-Hoechst), substituted aryloxy-3,3,3-trifluoro-2-propionic acids, esters and salts (U.S. Pat. No. 4,055,595-ICI), substituted hydroxyphenyl-piperidones (U.S. Pat. No. 4,024,267-Hoechst), and partially hydrogenated 1H-indeno-[1,2B]-pyridine derivatives (U.S. Pat. No. 3,980,656-Hoechst).

Montanari et al. (Pharmacological Research. Vol. 25, No. 1, 1992) disclose that use of metformin in amounts of 500 mg twice a day (b.i.d.) increased post-ischemia blood flow in a manner similar to 850 mg metformin three times a day (t.i.d.). Sirtori et al. (J. Cardiovas. Pharm., 6:914-923, 1984), disclose that metformin in amounts of 850 mg three times a day (t.i.d) increased arterial flow in patients with peripheral vascular disease.

The present invention provides the treatment of various diseases comprising GLP-1/linker/mTf fusion protein in combination with one or more therapeutic agents such as metformin. In one embodiment, the GLP-1/linker/mTf fusion protein in combination with metformin is used to treat diseases and conditions associated with abnormal blood glucose level, such as diabetes. Preferably, the GLP-1/linker/mTf fusion protein in combination with metformin is used to treat type II diabetes or obesity.

The present invention provides for the treatment of type 2 diabetes and/or obesity using a GLP-1/linker/mTf fusion protein of the invention in combination with a thiazolidinedione such as glitazone (rosiglitazone or pioglitazone). Thiazolidinediones reverse insulin resistance seen in type II diabetes.

Other therapeutic agents that may be used in combination with GLP-1/linker/mTf fusion protein of the present invention include but are not limited to DPPIV inhibitors, NEP inhibitors, sulfonylurea and sulfonylurea-like agents, Peroxisome Proliferator Activated Receptor (PPAR) gamma modulators, PPAR alpha modulators, Protein Tyrosine Phosphatase-1B inhibitors, Insulin Receptor Tyrosine Kinase activators, 11beta-hydroxysteroid dehydrogenase inhibitors, glycogen phosphorylase inhibitors, glucokinase activators, beta-3 adrenergic agonists, and glucagon receptor agonists.

Transgenic Animals

The production of transgenic non-human animals that contain a fusion construct with increased serum half-life increased serum stability or increased bioavailability of the instant invention is contemplated in one embodiment of the present invention. In some embodiments, lactoferrin may be used as the Tf portion of the fusion protein so that the fusion protein is produced and secreted in milk.

The successful production of transgenic, non-human animals has been described in a number of patents and publications, such as, for example U.S. Pat. No. 6,291,740 (issued Sep. 18, 2001); U.S. Pat. No. 6,281,408 (issued Aug. 28, 2001); and U.S. Pat. No. 6,271,436 (issued Aug. 7, 2001) the contents of which are hereby incorporated by reference in their entireties.

The ability to alter the genetic make-up of animals, such as domesticated mammals including cows, pigs, goats, horses, cattle, and sheep, allows a number of commercial applications. These applications include the production of animals which express large quantities of exogenous proteins in an easily harvested form (e.g., expression into the milk or blood), the production of animals with increased weight gain, feed efficiency, carcass composition, milk production or content, disease resistance and resistance to infection by specific microorganisms and the production of animals having enhanced growth rates or reproductive performance. Animals which contain exogenous DNA sequences in their genome are referred to as transgenic animals.

The most widely used method for the production of transgenic animals is the microinjection of DNA into the pronuclei of fertilized embryos (Wall et al., J. Cell. Biochem. 49:113 [1992]). Other methods for the production of transgenic animals include the infection of embryos with retroviruses or with retroviral vectors. Infection of both pre- and post-implantation mouse embryos with either wild-type or recombinant retroviruses has been reported (Janenich, Proc. Natl. Acad. Sci. USA 73:1260 [1976]; Janenich et al., Cell 24:519 [1981]; Stuhlmann et al., Proc. Natl. Acad. Sci. USA 81:7151 [1984]; Jahner et al., Proc. Natl. Acad Sci. USA 82:6927 [1985]; Van der Putten et al., Proc. Natl. Acad Sci. USA 82:6148-6152 [1985]; Stewart et al., EMBO J. 6:383-388 [1987]).

An alternative means for infecting embryos with retroviruses is the injection of virus or virus-producing cells into the blastocoele of mouse embryos (Jahner, D. et al., Nature 298:623 [1982]). The introduction of transgenes into the getline of mice has been reported using intrauterine retroviral infection of the midgestation mouse embryo (Jahner et al., supra [1982]). Infection of bovine and ovine embryos with retroviruses or retroviral vectors to create transgenic animals has been reported. These protocols involve the micro-injection of retroviral particles or growth arrested (i.e., mitomycin C-treated) cells which shed retroviral particles into the perivitelline space of fertilized eggs or early embryos (PCT International Application WO 90/08832 [1990]: and Haskell and Bowen, Mol. Reprod. Dev., 40:386 [1995]. PCT International Application WO 90/08832 describes the injection of wild-type feline leukemia virus B into the perivitelline space of sheep embryos at the 2 to 8 cell stage. Fetuses derived from injected embryos were shown to contain multiple sites of integration.

U.S. Pat. No. 6,291,740 (issued Sep. 18. 2001) describes the production of transgenic animals by the introduction of exogenous DNA into pre-maturation oocytes and mature, unfertilized oocytes (i.e., pre-fertilization oocytes) using retroviral vectors which transduce dividing cells (e.g., vectors derived from murine leukemia virus [MLV]). This patent also describes methods and compositions for cytomegalovirus promoter-driven, as well as mouse mammary tumor LTR expression of various recombinant proteins.

U.S. Pat. No. 6,281,408 (issued Aug. 28, 2001) describes methods for producing transgenic animals using embryonic stem cells. Briefly, the embryonic stem cells are used in a mixed cell co-culture with a morula to generate transgenic animals. Foreign genetic material is introduced into the embryonic stem cells prior to co-culturing by, for example, electroporation, microinjection or retroviral delivery. ES cells transfected in this manner are selected for integrations of the gene via a selection marker such as neomycin.

U.S. Pat. No. 6,271,436 (issued Aug. 7, 2001) describes the production of transgenic animals using methods including isolation of primordial germ cells, culturing these cells to produce primordial germ cell-derived cell lines, transforming both the primordial germ cells and the cultured cell lines, and using these transformed cells and cell lines to generate transgenic animals. The efficiency at which transgenic animals are generated is greatly increased, thereby allowing the use of homologous recombination in producing transgenic non-rodent animal species.

Gene Therapy

The use of modified transferrin fusion constructs for gene therapy wherein a modified transferrin protein or transferrin domain is joined to a therapeutic protein or peptide is contemplated in one embodiment of this invention. The modified transferrin fusion constructs with increased serum half-life or serum stability of the instant invention are ideally suited to gene therapy treatments.

The successful use of gene therapy to express a soluble fusion protein has been described. Briefly, gene therapy via injection of an adenovirus vector containing a gene encoding a soluble fusion protein consisting of cytotoxic lymphocyte antigen 4 (CTLA4) and the Fc portion of human immunoglubulin G1 was recently shown in Ijima et al. (Jun. 10, 2001) Human Gene Therapy (United States) 12/9:1063-77. In this application of gene therapy, a murine model of type II collagen-induced arthritis was successfully treated via intraarticular injection of the vector.

Gene therapy is also described in a number of U.S. patents including U.S. Pat. No. 6,225,290 (issued May 1, 2001); U.S. Pat. No. 6,187,305 (issued Feb. 13, 2001); and U.S. Pat. No. 6,140,111 (issued Oct. 31, 2000).

U.S. Pat. No. 6,225,290 provides methods and constructs whereby intestinal epithelial cells of a mammalian subject are genetically altered to operatively incorporate a gene which expresses a protein which has a desired therapeutic effect. Intestinal cell transformation is accomplished by administration of a formulation composed primarily of naked DNA, and the DNA may be administered orally. Oral or other intragastrointestinal routes of administration provide a simple method of administration, while the use of naked nucleic acid avoids the complications associated with use of viral vectors to accomplish gene therapy. The expressed protein is secreted directly into the gastrointestinal tract and/or blood stream to obtain therapeutic blood levels of the protein thereby treating the patient in need of the protein. The transformed intestinal epithelial cells provide short or long term therapeutic cures for diseases associated with a deficiency in a particular protein or which are amenable to treatment by overexpression of a protein.

U.S. Pat. No. 6,187,305 provides methods of gene or DNA targeting in cells of vertebrate, particularly mammalian, origin. Briefly, DNA is introduced into primary or secondary cells of vertebrate origin through homologous recombination or targeting of the DNA, which is introduced into genomic DNA of the primary or secondary cells at a preselected site.

U.S. Pat. No. 6,140,111 (issued Oct. 31, 2000) describes retroviral gene therapy vectors. The disclosed retroviral vectors include an insertion site for genes of interest and are capable of expressing high levels of the protein derived from the genes of interest in a wide variety of transfected cell types. Also disclosed are retroviral vectors lacking a selectable marker, thus rendering them suitable for human gene therapy in the treatment of a variety of disease states without the co-expression of a marker product, such as an antibiotic. These retroviral vectors are especially suited for use in certain packaging cell lines. The ability of retroviral vectors to insert into the genome of mammalian cells has made them particularly promising candidates for use in the genetic therapy of genetic diseases in humans and animals. Genetic therapy typically involves (1) adding new genetic material to patient cells in vivo or (2) removing patient cells from the body, adding new genetic material to the cells and reintroducing them into the body, i.e., in vitro gene therapy. Discussions of how to perform gene therapy in a variety of cells using retroviral vectors can be found, for example, in U.S. Pat. No. 4,868,116, issued Sep. 19, 1989, and U.S. Pat. No. 4,980,286, issued Dec. 25, 1990 (epithelial cells), WO 89/07136 published Aug. 10, 1989 (hepatocyte cells), EP 378,576 published Jul. 25, 1990 (fibroblast cells), and WO 89/05345 published Jun. 15, 1989 and WO/90/06997, published Jun. 28, 1990 (endothelial cells), the disclosures of which are incorporated herein by reference.

Kits Containing Transferrin Fusion Proteins

In a further embodiment, the present invention provides kits containing GLP-1/mTf or GLP-1/linker/mTf fusion proteins, which can be used, for instance, for the therapeutic or non-therapeutic applications. The kit comprises a container with a label. Suitable containers include, for example, bottles, vials, and test tubes. The containers may be formed from a variety of materials such as glass or plastic. The container holds a composition which includes a fusion protein that is effective for therapeutic or non-therapeutic applications, such as described above. The active agent in the composition is the therapeutic protein. The label on the container indicates that the composition is used for a specific therapy or non-therapeutic application, and may also indicate directions for either in vivo or in vitro use, such as those described above.

The kit of the invention win typically comprise the container described above and one or more other containers comprising materials desirable from a commercial and user standpoint, including buffers, diluents, filters, needles, syringes, and package inserts with instructions for use.

Without further description, it is believed that a person of ordinary skin in the art can, using the preceding description and the following illustrative examples, make and utilize the present invention and practice the claimed methods. For example, a skilled artisan would readily be able to determine the biological activity, both in vitro and in vivo, for the fusion protein constructs of the present invention as compared with the comparable activity of the therapeutic moiety in its unfused state. Similarly, a person skined in the art could readily determine the serum half life and serum stability of constructs according to the present invention. The following working examples therefore, specifically point out the preferred embodiments of the present invention, and are not to be construed as limiting in any way the remainder of the disclosure.

### EXAMPLES

### Example 1

### GLP-1/Transferrin Fusion Protein

GLP-1 is a peptide that regulates insulin secretion. It possesses anti-diabetic activity in human subjects suffering diabetes, especially type II diabetes. Like other peptides, GLP-1 has a short plasma half-life in humans. The present invention provides fusion proteins with GLP-1 fused to mTf with increased half-life and pharmaceutical compositions of such fusion proteins for the treatment of diseases associated with abnormal glucose levels.

The present invention also provides fusion proteins comprising an GLP-1 analog and mTF. In one embodiment of the invention, the GLP-1 analog comprises an additional His residue at the N-terminus. The His residue could be added to the N-terminus of GLP-1 or inserted after the His residue at the N-terminus of GLP-1. In another embodiment, the GLP-1 analog comprises an amino acid substitution at position 2. For example, the Ala in GLP-1(7-36) or GLP-1(7-37) peptide is substituted with another amino acid.

In this example, the steps for producing a GLP-1/mTf fusion protein are described. The same steps may be used to generate transferrin fusion proteins with analogs of the GLP-1 peptides.

To produce the GLP-1/mTf fusion protein, the amino acid sequence of GLP-1(7-36) and GLP-1(7-37) may be used.

| | |
|---|---|
| haegtftsdvssylegqaakefiawlvkgr | (SEQ ID NO: 4) |
| haegtftsdvssylegqaakefiawlvkgrg | (SEQ ID NO: 6) |

For example, the peptide sequence of GLP-1(7-36) may be back translated into DNA and codon optimized for yeast:

| | |
|---|---|
| catgctgaaggtacttttacttctgatgtttctt | (SEQ ID NO: 69) |
| h a e g t f t s d v s s | (SEQ ID NO: 4) |
| cttatttggaaggtcaagctgctaaagaatttat | |
| y l e g q a a k e f i | |
| tgcttggttggttaaaggtaga | |
| a w l v k g r | |

The primers were specifically designed to form 5′ XbaI and 3′ KpnI sticky ends after annealing and to enable direct ligation into XbaI/KpnI cut pREX0052, just 5′ of the end of the leader sequence and at the N-terminus of mTf. Alternatively, other sticky ends may be engineered for ligations into other vectors.

| | |
|---|---|
| | XbaI |
| | −+−−−−− |
| 1 | aggtctctag agaaaaggca tgctgaaggt acttttactt ctgatgtttc ttcttatttg |
| | tccagagatc tcttttccgt acgacttcca tgaaaatgaa gactacaaag aagaataaac |
| | >>......FL.......>> |
| | r s l e k r (SEQ ID NO: 52) |
| | >>..................GLP-1....................> |
| | (SEQ ID NO: 4) h a e g t f t s d v s s y l |
| | KpnI |
| | −−−−−−+ |
| 61 | gaaggtcaag ctgctaaaga atttattgct tggttggtta aaggtagggt acctgata |
| | cttccagttc gacgatttct taaataacga accaaccaat ttccatccca tggactat |
| | >......................GLP-1......................>> |
| | e g q a a k e f i a w l v k g r |
| | >>..mTf..>> |
| | v p d |

Top strand: SEQ ID NO: 67
Bottom strand: SEQ ID NO: 68
Top strand primer: P0056 (nucleotides 7-112 of SEQ ID NO: 67)
Bottom strand primer: P0057 (nucleotidas 9-108 of SEQ ID NO: 68)

After annealing and ligation, the clones were sequenced to confirm correct insertion. This vector was designated pREX0094. The cassette was cut out of pREX0094 with NotI and sub-cloned into NotI cut yeast vector, pSAC35, to make pREX0100.

This plasmid was then electroporated into the host Saccharomyces cerevisiae yeast strains and transformants selected for leucine prototrohy on minimal media plates. Expression as determined by growth in liquid minimal media and analysis of supernatant by SDS-PAGE, western blot, and ELISA.

### Example 2

### The Addition of Linkers to Improve Activity

The addition of linker or spacer between GLP-1 and mTf was investigated as a way to improve activity of a GLP-1 mTf fusion based on the hypothesis that steric hindrance from the mTf carrier could reduce the ability of GLP-1 to bind its receptor. Thus, it was hypothesized that steric hindrance could be reduced and activity recovered by inserting a linker to increase the distance between GLP-1 and mTf. Three different types of linkers were tested: flexible linkers, the short flexible linkers and the rigid linkers.

The long flexible linkers tested were a (SGGG)₃ repeat and a linker based on the GLP-2 sequence (“GLP-2 linker”). The (SGGG)₃ repeat and similar sequences have been used as linkers, for example, in single chain Fv fragments linking Vh and Vl together. The GLP-2 linker was tested because GLP-2 naturally occurs closely linked to GLP-1 in the propeptide that is processed to give glucagon, GLP-1 and GLP-2. The intervening peptide sequence that is cleaved to give the two GLP peptides was deleted as was the N-terminal residue on GLP-2 rendering it inactive in the event that GLP-1 was cleaved from the linker by some means. Further, because GLP-2 is a natural sequence, the likelihood of immunogenicity would be presumed to be reduced.

The short flexible linkers tested were S, SS and SSG. This class of linkers was tested to determine the effect of flexibility at the junction of the two moieties in the absence GLP-1 spacing away from mTf.

The rigid linkers, i.e., substantially non-helical linkers, were designed based upon the idea that it was not mobility but clear separation of the two moieties that was required for GLP-1 to find its receptor. Using the naturally occurring sequence between N and C lobes of Tf, i.e., between Cys331 and Cys339 (PEAPTDE), made use of a sequence seen in high abundance in the circulatory system and thus with reduced concern for its potential to be immunogenic. The proline residues within this sequence along with that at the second residue position in mTf would be expected to result in a peptide with little flexibility. The linkers created were PE, PEA, PEAPTD, (PEAPTD)₂, (PEAPTD)₃ and (PEAPTD)₄.

The hinge region from human IgGl as investigated as an alternative naturally occurring high abundance linker sequence. The IgG hinge is a region of sequence joining the constant (Fc) and variable region (Vh) of the human IgGl heavy chain. In the native protein, the hinge allows for flexibility of the IgG molecule during antigen binding. This sequence has been used de facto in linking peptides or proteins at the N-terminus of Fc fusions. It has also been used in recombinant proteins to engineer a flexible region between two distinct protein domains (Doyle el al. 2003 Regulatory Peptides, 114, 153-158). The sequence normally cited as the IgG hinge is EPKSCDKTHTCPPCP (residues 224-238) (SEQ ID NO.: 88) and includes the cysteine that forms a disulphide bond with a light chain and the two cysteines that form disulphide bonds with the other heavy chain Fc region of an antibody.

For the desired linker length, this sequence was extended to 25 residues, VEPKSCDKTHTCPPCPAPELLGGPS (SEQ ID NO.: 124). The linker was modified by mutating cysteine residues to serine to prevent disulphide bond formation of free cysteines (VEPKSSDKTHTSPPSPAPELLGGPS (SEQ ID NO.: 89)). The cysteine residues can likewise be substituted with alanines to produce VEPKSADKTHTAPPAPAPELLGGPS (SEQ ID NO.: 117). Further, the cysteine residues and serine residues can be substituted with alanine to produce VEPKAADKTHTAPPAPAPELLGGPA (SEQ ID NO.: 125). Both SEQ ID NOS.: 117 and 125 exhibit reduced O-glycosylation as a result of the removal of the serine residues. The hinge sequences described in this paragraph can be used in conjunction with a single PEAPTD sequence or PEAPTD multimer, e.g., (PEAPTD)₂, to further extend the linker.

The C-terminal sequence of Exendin-4 plays a major role in GLP-1 receptor binding affinity and addition of the 9aa C-terminal sequence, SSGAPPPS (SEQ ID NO.: 15), to GLP-1 peptide increased its affinity for the GLP-1 receptor (Heuser el al. 2004 Int. J. Cancer 110: 386-394). This sequence was used as a linker in conjunction with the IgG hinge.

A number of linkers were tested. The results are summarized below.

GLP-1| Linker |mTf
(SGGG)₃
GLP-2
S
SS
SSG
PE
PEA
PEAPTD
(PEAPTD)₂
(PEAPTD)₃
(PEAPTD)₄
CEx
IgG hinge
PEAPTD IgG hinge
CEx IgG hinge

| | |
|---|---|
| (SGGG)₃ | SGGGSGGGSGGG |
| GLP-2 | HADGSFSDEMNTILDNLAARDFINWLIQTKITDR |
| | (SEQ ID No. 90) |
| | ADGSFSDEMNTILDNLAARDFINWLIQTKITDR |
| | (SEQ ID No. 91) |
| PEAPTD | based on linker between N and C lobes of |
| | Tf between Cys331 and Cys339, CPEAPTDEC. |
| CEx | C-terminal extension to Exendin-4. |
| | SSGAPPPS (SEQ ID NO.: 15) |
| IgG hinge | Linker between heavy chain variable and |
| | Fc region of IgG antibody. |
| | VEPKSSDKTHTSPPSPAPELLGGPS |
| | (SEQ ID NO.: 89) |

Underlined serine residues mutated from cysteine in native sequence.

Construction of Long Flexible Linkers.

The (SGGG)₃ (SEQ ID NO.: 16) linker construct pREX0216 [ΔL GLP-1(7-36) (SGGG)₃ mTf] was made by designing primers that when annealed together would have XbaI/KpnI overhangs: P0281, P0282, P0283 and P028.

(SEQ ID NO.: 92)

P0281
CTAGAGAAAAGGCATGCTGAAGGTACTTTTACTTCT
GATGTTTCTTCTTATTTGGAAGGTCAAGCTGCTAAA
G

(SEQ ID NO.: 93)

P0282
AATTTATTGCTTGGTTGGTTAAAGGTAGGTCTGGTG
GTGGTTCTGGTGGTGGTTCTGGTGGTGGTGGTAC

(SEQ ID NO.: 94)

P0283
CACCACCACCAGAACCACCACCAGAACCACCACCAG
ACCTACCTTTAACCAACCAAGCAATAAATTCTTTAG
CAGCTTGAC

(SEQ ID NO.: 95)

P0284
CTTCCAAATAAGAAGAAACATCAGAAGTAAAAGTAC
CTTCAGCATGCCTTTTCT

These were annealed by mixing 10 μL of each primer (20 pmol/μL) in a tube containing 10 μL 10×PCR buffer (without MgCl₂). The reaction was carried out at 68° C. for 5 minutes, 37° C. for 10 minutes, and 20° C. for 10 minutes. The annealed product was cleaned up (Qiagen Gel Extraction Kit, PCR protocol) and the product cloned into XbaI/KpnI digested pREX0095 creating pREX0216. Clones were DNA sequenced to check if the insert was correct. A correct clone was obtained and the expression cassette was extracted by NotI/ScaI digest and cloned into NotI digested and SAP treated pSAC35 creating pREX0217. Clones were screened for inserts in the same orientation as the LEU2 gene using XbaI/SalI digest. pREX0217 DNA was transformed into the Saccharomyces cerevisiae strain Control Strain (see WO 05061718) by electroporation with a BioRad Gene Pulser in 2 mm cuvettes and plated onto BMM/S plates. A yeast stock of this construct was made: Y0160. Shake flask cultures yielded 52 ng/mL by ELISA.

The (SGGG)₃ linker construct pREX0217 was fermented, F0079, giving productivity of only 0.218 μg/mL by ELISA (Table 1). However, the activity was greatly improved with an EC₅₀ of 2.274 nM (cAMP 060204-1) compared to around 0.6 nM for GLP-1 peptide.

The ΔL GLP-1(7-36) GLP-2 linker mTf construct pREX0213 was made by designing linkers that could be annealed together: P0273, P0274, P0275, P0276, P0277, P0278, P0279 and P0280 and cloned into pREX0095.

(SEQ ID NO.: 24)

P0273
CTAGGTCTCTAGAGAAAAGGCATGC

(SEQ ID NO.: 25)

P0274
AAATAAGAAGAAACATCAGAAGTAAAAGTACCTTCA
GCATGCCTTTTCTCTAGAGACCTAG

(SEQ ID NO.: 26)

P0275
TGAAGGTACTTTTACTTCTGATGTTTCTTCTTATTT
GGAAGGTCAAGCTGCTAAAGAATTTATTGCTTGGTT
G

(SEQ ID NO.: 27)

P0276
AGAAAGAACCATCAGCATGCCTACCTTTAACCAACC
AAGCAATAAATTCTTTAGCAGCTTGACCTTCC

(SEQ ID NO.: 28)

P0277
GTTAAAGGTAGGCATGCTGATGGTTCTTTCTCTGAT
GAGATGAACACCATTCTTGATAATCT

(SEQ ID NO.: 29)

P0278
GTCTGAATCAACCAGTTTATAAAGTCCCTGGCGGCA
AGATTATCAAGAATGGTGTTCATCTCATCAG

(SEQ ID NO.: 30)

P0279
TGCCGCCAGGGACTTTATAAACTGGTTGATTCAGAC
CAAAATCACTGACAGGGTACCTGAT

(SEQ ID NO.: 31)

P0280
ATCAGGTACCCTGTCAGTGATTTTG

These primers basically recreated GLP-1 with the GLP-2 linker attached and were annealed by mixing 10 μL of each primer (20 pmol/μL) in a tube containing 10 μL 10×PCR buffer (without MgCl₂). The reaction was carried out at 68° C. for 5 minutes. 37° C. for 10 minutes, and 20° C. for 10 minutes. To complete the annealing 5 μL of T4 DNA Ligase was added to the reaction and incubated for 2 hours at room temperature. A PCR reaction was set up using P0273 and P0280 as the outer primers to amplify the annealed product. The reaction conditions for the PCR were: 1 cycle of 94° C. for 1 minute, 25 cycles of 94° C. for 40 seconds, 55° C. for 40 seconds, and 72° C. for 1 minute, followed by a final extension at 72° C. for 7 minutes. The resulting PCR product was digested XbaI/KpnI and cloned into XbaI/KpnI digested pREX0095 to create pREX0213. Clones were DNA sequenced between the two restriction sites to check that the sequence was correct. Once a correct clone was obtained, the expression cassette was extracted by NotI/ScaI digest and cloned into NotI digested and SAP treated pSAC35 to create pREX0214. Clones were screened for inserts in the same orientation as the LEU2 gene using XbaI/Sa/I digest. pREX0214 DNA was transformed into the Saccharomyces cerevisiae Control Strain (WO 05/061718) by electroporation with a BioRad Gene Pulser in 2 mm cuvettes and plated onto BMM/S plates. Colonies were set up to grow in BMM/S shake flask culture, (at 30° C., 200 rpm) and productivity was determined to be 5 ng/mL by anti-Tf ELISA. A yeast stock of this construct, Y0172, was made.

The GLP-2 linker constructs pREX0214 was fermented, F0080. It has a productivity of 9 μg/mL (Table 1). As with the (SGGG)₃ linker construct the activity was vastly improved with an EC₅₀ of 5.9.

TABLE 1
Results of Long Flexible Linker Fermentations

| | | Fermen- | ELISA | cAMP | Activity |
|---|---|---|---|---|---|
| Plasmid | Construct | tation | ug/mL | Assay # | (nM) |
| pREX0217 | ΔL GLP-1 | F0079 | 0.281 | cAMP | 2.274 |
| | (7-36) (SGGG)₃ | | | 060204-1 | |
| | linker mTf | | | | |
| pREX0214 | ΔL GLP-1 | F0080 | 9 | cAMP | 5.9 |
| | (7-36) GLP-2 | | | 051804-1 | |
| | linker-mTf | | | | |

Construction of Short Flexible Linkers

The short flexible linkers S, SS, and SSG were made in various constructs.

TABLE 2
Short Flexible Linker Constructs

| Plasmid | Plasmid | |
|---|---|---|
| (pBST+) | (pSAC 35) | Construct |
| pREX0506 | pREX0507 | nL GLP-1 (7-37; K34N) SS mTf |
| pREX0508 | pREX0509 | nL GLP-1 (7-37; K34N) SSG mTf |

The construction of the short flexible linker variants was achieved by designing overlapping PCR primers.

(SEQ ID NO.: 32)

External Primers

| | | |
|---|---|---|
| P0012 | CATGATCTTGGCGATGCAGTC | |
| P0025 | AGCGGATAACAATTTCACACAGGA | (SEQ ID NO.: 33) |

Mutagenic Primers:
pREX0506 [nL GLP-1 (7-37; K34N) SS mTf]

| | | |
|---|---|---|
| P0538 | GTTGGTTAATGGTAGGGGTTCTTCTGTAC | (SEQ ID NO.: 34) |
| | CTGATAAAAC | |
| P0539 | GTTTTATCAGGTACAGAAGAACCCCTACC | (SEQ ID NO.: 35) |
| | ATTAACCAAC | |

pREX0508 [nL GLP-1 (7-37; K34N) SSG mTf]

| | | |
|---|---|---|
| P0540 | GTTGGTTAATGGTAGGGGTTCTTCTGGTG | (SEQ ID NO.: 36) |
| | TACCTGATAAAAC | |
| P0541 | GTTTTATCAGGTACACCAGAAGAACCCCT | (SEQ ID NO.: 37) |
| | ACCATTAACCAAC | |

The linkers were inserted between the GLP-1 variant and mTf through a first round of PCR combining P0025 with the reverse mutagenic primer, P0012 with the forward mutagenic primer followed by a second round where the two first round products are joined with P0025 and P0012. The reaction conditions for the first round were 15 cycles of 94° C. for 1 minute, 50° C. for 1 minute, and 72° C. for 1 minute, followed by a final extension of 10 minutes at 72° C. The second round reaction conditions were 1 cycle of 94° C. for 1 minute, 25 cycles of 94° C. for 30 seconds. 55° C. for 30 seconds, and 72° C. for 1 minute, followed by a final extension at 72° C. for 7 minutes. PCR products were then digested AflII/BamHI and cloned into pREX0052 cut AflII/BamHI. Clones were DNA sequenced between the restriction sites to confirm correct insertion. Correct clones were chosen, and expression cassettes were extracted via NotI/PvuI digest. The expression cassettes were cloned into NotI digested and SAP treated pSAC35. The clones were screened for insertion in the same orientation as the LEU2 gene using XbaI/SalI digest. DNA was transformed into S. cerevisiae strain Strain A (WO 05/061718) by electroporation with a BioRad Gene Pulser in 2 mm cuvettes. Stocks were made of the constructs and DNA sequenced to confirm identity.

The single S linker, as in the construct pREX0456, resulted in a productivity of 37 μg/mL from fermentor (F0140). The productivities of the SS linker (pREX0507 and SSG linker (pREX0509) from fermentor were 1200 μg/mL (F151 & 158) and an average of 1346 μg/mL (F149, 152, 153 & 156) respectively (Table 3). Although the productivities were good, the activity was about the same as that for a construct without a linker (pREX0505 F150, 163, 164, 169, 170).

TABLE 3
Fermentation Results: Short Flexible Linker Constructs

| | | Fermen- | ELISA | cAMP | Activity |
|---|---|---|---|---|---|
| Plasmid | Construct | tation | μg/mL | Assay | (nM) |
| pREX0507 | nL GLP-1(7-37; | F0158 | 1200 | cAMP | 156.9 |
| | K34N) SS mTf | | | 061104-1 | |
| pREX0509 | nL GLP-1(7-37; | F0149 | 2800 | cAMP | 73.8 |
| | K34N) SSG mTf | F0152 | 724 | 060204-1 | |
| | | F0153 | 1200 | | |
| | | F0156 | 662 | | |

Construction of Rigid Linkers

The rigid linker constructs made based upon the sequence linking the N and C lobes of Tf are shown.

TABLE 4
Rigid Linker Constructs-Transferrin linkers

| Plasmid | Plasmid | |
|---|---|---|
| (pBST+) | (pSAC35) | Construct |
| pREX0517 | pREX0518 | nL GLP-1(7-37; K34N) PEA mTf |
| pREX0519 | pREX0520 | nL GLP-1(7-37; K34N) PEAPTD mTf |
| pREX0521 | pREX0522 | nL GLP-1(7-37; K34N) PE mTf |
| | | (V1A) |
| pREX0566 | pREX0567 | nL GLP (7-37; K34A) PEAPTD mTf |
| pREX0568 | pREX0569 | nL GLP-1 (7-37; A8G, K34A) |
| | | PEAPTD mTf |
| pREX0570 | pREX0571 | nL GLP-1 (7-37; A8G, G22E, |
| | | K34A) PEAPTD mTf |
| pREX0572 | pREX0573 | nL GLP-1 (7-37; A8G, G22E, |
| | | K34A) (PEAPTD)₂ mTf |
| pREX0574 | pREX0575 | nL GLP-1 (7-37; A8G, G22E, |
| | | K34A) (PEAPTD)₃ mTf |
| pREX0576 | pREX0577 | nL GLP-1 (7-37; A8G, G22E, |
| | | K34A) (PEAPTD)₄ mTf |
| pREX0584 | pREX0585 | nL GLP-1 (7-37; A8G, K34A) |
| | | (PEAPTD)₂ mTf |

In addition to insertion of the PEAPTD based linkers, two additional changes were made to the GLP-1 construct. An A8G mutation was incorporated for DPP IV resistance. A G22E mutation was incorporated to remove a potential kink in the α-helix backbone of GLP-1 which has been implicated in possible increased activity of GLP-1 analogues such as Exendin-4.

These variants were made by using mutagenic PCR primers in two rounds of PCR.

(SEQ ID NO.: 70)

External Primers

| | | |
|---|---|---|
| P0012 | CATGATCTTGGCGATGCAGTC | |
| P0025 | AGCGGATAACAATTTCACACAGGA | (SEQ ID NO.: 38) |

Mutagenic Primers
pREX0517 [nL GLP-1 (7-37; K34N) PEA mTf]

| | | |
|---|---|---|
| P0550 | AATGGTAGGGGTCCAGAAGCTGTACCTGA | (SEQ ID NO.: 39) |
| | T | |
| P0551 | ATCAGGTACAGCTTCTGGACCCCTACCAT | (SEQ ID NO.: 40) |
| | T | |

pREX0519 [nL GLP-1 (7-37; K34N) PEAPTD mTf]

| | | |
|---|---|---|
| P0552 | ATGGTAGGGGTCCAGAAGCTCCAACTGAT | (SEQ ID NO.: 41) |
| | GTACCTGATAA | |
| P0553 | TTATCAGGTACATCAGTTGGAGCTTCTGG | (SEQ ID NO.: 42) |
| | ACCCCTACCAT | |

pREX0521 [nL GLP-1 (7-37; K34N) PE mTf (V1A)]

| | | |
|---|---|---|
| P0554 | GTAGGGGTCCAGAAGCTCCTGATAAAAC | (SEQ ID NO.: 43) |
| P0555 | GTTTTATCAGGAGCTTCTGGACCCCTAC | (SEQ ID NO.: 44) |

pREX0566 [nL GLP (7-37; K34A) PEAPTD mTf]
pREX0568 [nL GLP-1 (7-37; A8G, K34A) PEAPTD mTf]
pREX0570 [nL GLP-1 (7-37; A8G, G22E, K34A) PEAPTD
mTf]

| | | |
|---|---|---|
| P0661 | TTGGTTGCTGGTAGGGGTCCAGAAGCTC | (SEQ ID NO.: 45) |
| | CAACTGATGTACCTGATAAAAC | |
| P0662 | GTTTTATCAGGTACATCAGTTGGAGCTT | (SEQ ID NO.: 46) |
| | CTGGACCCCTACCAGCAACCAA | |

pREX0572 [nL GLP-1 (7-37; A8G, G22E, K34A)
(PEAPTD)₂ mTf]
pREX0584 [nL GLP-1 (7-37; A8G, K34A)
(PEAPTD)₂ mTf]

| | | |
|---|---|---|
| P0663 | TTGGTTGCTGGTAGGGGTCCAGAAGCTC | (SEQ ID NO.: 47) |
| | CAACTGATCCAGAAGCTCCAACTGATGT | |
| | ACCTGATAAAACTG | |
| P0664 | CAGTTTTATCAGGTACATCAGTTGGAGC | (SEQ ID NO.: 48) |
| | TTCTGGATCAGTTGGAGCTTCTGGACCC | |
| | CTACCAGCAACCAA | |

pREX0574 [nL GLP-1 (7-37; A8G, G22E, K34A)
(PEAPTD)₃ mTf]

| | | |
|---|---|---|
| P0665 | GGTTGGTTGCTGGTAGGGGTCCAGAAGC | (SEQ ID NO.: 49) |
| | TCCAACTGATCCAGAAGCTCCAACTGAT | |
| | CCAGAAGCTCCAACTGATGTACCTGATA | |
| | AAACTGTG | |
| P0666 | CACAGTTTTATCAGGTACATCAGTTGGA | (SEQ ID NO.: 50) |
| | GCTTCTGGATCAGTTGGAGCTTCTGGAT | |
| | CAGTTGGAGCTTCTGGACCCCTACCAGC | |
| | AACCAACC | |

pREX0576 [nL GLP-1 (7-37; A8G, G22E, K34A)
(PEAPTD)₄ mTf]

| | | |
|---|---|---|
| P0721 | TTGGTTGCTGGTAGGGGTCCAGAAGCTC | (SEQ ID NO.: 76) |
| | CAACTGATCCAGAAGCTCCAACTGATCC | |
| | AGAAGCTCCAACTGATCCAGAAGCTCCA | |
| | ACTGATGTACCTGATAAAACTGTGAG | |
| P0722 | CTCACAGTTTTATCAGGTACATCAGTTG | (SEQ ID NO.: 51) |
| | GAGCTTCTGGATCAGTTGGAGCTTCTGG | |
| | ATCAGTTGGAGCTTCTGGATCAGTTGGA | |
| | GCTTCTGGACCCCTACCAGCAACCAA | |

In the first round of PCR, the forward mutagenic primer was combined with P0012 and the reverse mutagenic primer was combined with P0025. In the second round of PCR, the two products are joined together with P0025 and P0012. The reaction conditions for the first round were 15 cycles of 94° C. for 1 minute, 50° C. for 1 minute, and 72° C. for 1 minute, followed by a final extension of 10 minutes at 72° C. The second round reaction conditions were 1 cycle of 94° C. for 1 minute, 25 cycles of 94° C. for 30 seconds, 50° C. for 30 seconds, and 72° C. for 1 minute, followed by a final extension at 72° C. for 7 minutes. PCR products were then digested AflII/BamHI and cloned into pREX0052 cut AflII/BamHI. Clones were DNA sequenced between the restriction sites to confirm correct insertion. Correct clones were chosen, and expression cassettes were extracted via NotI/Pvul digest. The expression cassettes were cloned into pSAC35. The clones were screened for insertion in the same orientation as the LEU2 gene using XbaI/SalI digest. DNA was transformed into into S. cerevisiae strain Strain A (see WO 05/061718) by electroporation with a BioRad Gene Pulser in 2 mm cuvettes. Stocks were made of the constructs and DNA sequenced to confirm identity.

TABLE 5
Yeast Stocks of Rigid Linkers-Transferrin Linkers

| Plasmid | Construct | Yeast Stock |
|---|---|---|
| pREX0518 | nL GLP-1(7-37; K34N) | Y0342, Y0350 |
| | PEA mTf | |
| pREX0520 | nL GLP-1(7-37; K34N) | Y0343, Y0351 |
| | PEAPTD mTf | |
| pREX0522 | nL GLP-1(7-37; K34N) PE | Y0344 |
| | mTf (V1A) | |
| pREX0567 | nL GLP (7-37; K34A) PEAPTD | Y0365, Y0382 |
| | mTf | |
| pREX0569 | nL GLP-1 (7-37; A8G, K34A) | Y0375, Y0378 |
| | PEAPTD mTf | |
| pREX0571 | nL GLP-1 (7-37; A8G, G22E, | Y0376 |
| | K34A) PEAPTD mTf | |
| pREX0573 | nL GLP-1 (7-37; A8G, G22E, | Y0379 |
| | K34A) (PEAPTD)₂ mTf | |
| pREX0575 | nL GLP-1 (7-37; A8G, G22E, | Y0377 |
| | K34A) (PEAPTD)₃ mTf | |
| pREX0577 | nL GLP-1 (7-37; A8G, G22E, | Y0381, Y0383 |
| | K34A) (PEAPTD)₄ mTf | |
| pREX0585 | nL GLP-1 (7-37; A8G, K34A) | Y0388, Y0405, |
| | (PEAPTD)2 mTf | Y0412 |

Fermentations with the PEA or PEAPTD based linker constructs all resulted in good productivity of around 1 g/L by ELISA (Table 6). The PEA linker construct, pREX0518, had a potency of 375 nM which was to several times lower than the construct without the linker (pREX0505-2162 nM). The (PEAPTD)₂ linker increased potency and productivity. Combination of A8G and (PEAPTD)₂ resulted in the combination of highest productivity with potency closest to that of GLP-1 peptide in the cAMP assay.

Variants with the single PEAPTD linker were made and fermented. The (PEAPTD)₂ linker was made with two construct variants. One was fermented, pREX0585, yielding productivity of 1900 μg/mL by ELISA and potency of 3 nM (Table 6).

TABLE 6
Fermentation Results: Rigid (Transferrin) Linker Constructs

| | | Fermen- | ELISA | cAMP | Activity |
|---|---|---|---|---|---|
| Plasmid | Construct | tation | μg/mL | Assay | (nM) |
| pREX0518 | nL GLP-1(7-37; | F0160 | 973 | cAMP | 375 |
| | K34N) | | | 061604-1 | |
| | PEA mTf | | | | |
| pREX0520 | nL GLP-1(7-37; | F0159 | 1900 | cAMP | 85 |
| | K34N) | | | 061604-1 | |
| | PEAPTD mTf | | | | |
| pREX0567 | nL GLP-1(7-37; | F0183 | 1300 | cAMP | 22 |
| | K34A) | | | 061104-1 | |
| | PEAPTD mTf | | | | |
| pREX0569 | nL GLP-1(7-37; | F0185 | 1200 | na | na |
| | A8G; K34A) | | | | |
| | PEAPTD mTf | | | | |
| pREX057l | nL GLP-1(7-37; | F0184 | 739 | na | na |
| | A8G; G22E; | | | | |
| | K34A) PEAPTD | | | | |
| | mTf | | | | |
| pREX0585 | nL GLP-1(7-37; | F0249 | 1900 | PD6 | 3 |
| | A8G; K34A) | | | | |
| | (PEAPTD)₂ mTf | | | | |

Construction of IgG Hinge and Exendin-4 Linkers

A two-step PCR protocol was used to generate pREX0556 though pREX0559 with the primers listed in Table 7.

TABLE 7

Primers used to generate IgG and CEx
linker constructs pREX0556-559.

| | |
|---|---|
| Primer | Sequence |

| | |
|---|---|
| P0676 | ATGGTGGCGAAGTATGAGTTTTATCCGACGATTTTGGTTCAACA |
| | CCCCTACCAGCAACCAACCAAGCAATAAATTCTTTAGCAG |
| | (SEQ ID NO.:52) |
| P0677 | GTCGGATAAAACTCATACTTCGCCACCATCGCCAGCTCCAGAAT |
| | TGTTGGGTGGTCCATCGGTACCTGATAAAACTGTGAGATGGTG |
| | (SEQ ID NO.:53) |
| P0678 | CGAAGTATGAGTTTTATCCGACGATTTTGGTTCAACCGATGGTG |
| | GTGGAGCACCCGACGAACCCCTACCAGCAACCAACCAAGCAATA |
| | AATTCTTTAGCAG (SEQ ID NO.:54) |
| P0679 | GTTGAACCAAAATCGTCGGATAAAACTCATACTTCGCCACCATC |
| | GCCAGCTCCAGAATTGTTGGGTGGTCCATCGGTACCTGATAAAA |
| | CTGTGAGATG (SEQ ID NO.:55) |
| P0680 | GATGGTGGCGAAGTATGAGTTTTATCCGACGATTTTGGTTCAAC |
| | ATCAGTTGGAGCTTCTGGACCCCTACCAGCAACCAACCAAGCAA |
| | TAAATTCTTTAGCAG (SEQ ID NO.:56) |
| P0681 | CAAAATCGTCGGATAAAACTCATACTTCGCCACCATCGCCAGCT |
| | CCAGAATTGTTGGGTGGTCCATCGGTACCTGATAAAACTGTGAG |
| | ATGGTG (SEQ ID NO.:57) |
| P0719 | CTTTAGCAGCTTGTTCTTCCAAATAAGAAGAAACATCAGAAGTA |
| | AAAGTACCTTCACCATGCGCCAGACACAGCCCCA |
| | (SEQ ID NO.:58) |
| P0720 | GTACTTTTACTTCTGATGTTTCTTCTTATTTGGAAGAACAAGCT |
| | GCTAAAG (SEQ ID NO.:59) |
| P0723 | CCGTTATATTGGAGTTCTTCC (SEQ ID NO.:60) |
| P0724 | ACTGCTTTCTCAAGAGGTTTAC (SEQ ID NO.:61) |
| P0745 | GTTGAACCAAAATCGTCGGATAAAACTCATACTTCGCCACCAT |
| | (SEQ ID NO.:62) |

pREX0556 contains GLP-1 (7-37;A8G;G22E;K34A) fused to the N-terminus of transferrin with an IgG hinge linker (VEPKSSDKTHTSPPSPAPELLGGPS). In the first step, the 5′segment was amplified from pREX0555 with primers P0723 and P0676 and the 3′ segment was amplified from pREX0523 with P0677, P0724, and P0745. Since P0677 was a relatively long primer (87 bp), the shorter primer P0745 was included to improve the efficiency of amplification. The reaction conditions were 94° C. for 1 min., 15 cycles 94° C. for 40 seconds, 55° C. for 40 seconds and 72° C. for 1 minute followed by a 7 minute extension at 72° C. The 5′ and 3′ segments were joined by amplification with P0723 and P0724 using the conditions above for 18 cycles. The same PCR conditions were used for all four constructs. The product was digested AflII/BamHI, cloned into the AflII/BamHI sites of pREX0549, and sequenced. The GLP-1 transferrin fusion expression cassette was excised from pREX0549 with a NoTI/PvuI digest, cloned into the NotI site of pSAC35, and DNA sequenced.

pREX0557 contains GLP-1(7-37;A8G;G22E;K34A) fused to the N-terminus of transferrin with the C-terminal end of Exendin-4 (CEx, SSGAPPPS) and the IgG hinge as linkers. In the first step, the 5′segment was amplified from pREX0555 with primers P0723 and P0678 and the 3′ segment was amplified from pREX0523 with P0679, P0724, and P0745. The 5′ and 3′ segments were joined by amplification with P0723 and P0724. The product was cloned into pCR2.1, DNA sequenced, and then digested AflII/BamHI and cloned into pREX0549. The GLP-1 transferrin fusion expression cassette was excised from pREX0549 by a NotI/PvuI digest and cloned into the NotI site of pSAC35, and DNA sequenced.

pREX0558 contains GLP-1 (7-37;A8G;G22E;K34A) fused to the N-terminus of transferrin with PEAPTD and IgG hinge linkers. In the first step, the 5′segment was amplified from pREX0555 with primers P0723 and P0680 and the 3′ segment was amplified from pREX0523 with P0681, P0724, and P0745. The 5′ and 3′ segments were joined by amplification with P0723 and P0724. The product was cloned into pCR2.1, DNA sequenced, and then digested AflII/BamHI and cloned into pREX0549. The GLP-1 transferrin expression cassette was excised from pREX0549 with a NotI/PvuI digest, cloned into the NotI site of pSAC35, and DNA sequenced.

pREX0559 contains GLP-1 (7-37;A8G;G22E;K34A) fused to the N-terminus of transferrin with a CEx linker. In the first step, the 5′segment was amplified from pREX0555 with primers P0723 and P0719 and the 3′ segment was amplified from pREX0523 with P0720 and P0724. The 5′ and 3′ segments were joined by amplification with P0723 and P0724. The product was digested AflII/BamHI, cloned into pREX0549, and sequenced. The GLP-1 transferrin expression cassette was excised from pREX0549 with a NotI/PvuI digest, cloned into the NotI site of pSAC35, and DNA sequenced.

Constructs were transformed into Saccharomyces cerevisiae strain Strain A (see WO 05/061718) by electroporation with a BioRad Gene Pulser in 2 mm cuvettes and plated onto BMM/S medium. Transformants were grown on BMM/S for four days and then patched onto BMM/S plates containing anti-transferrin polyclonal serum (Calbiochem cat. no. 616423). After 4 days, halos of precipitated antibody/transferrin complexes were visible for all four constructs. Transformed clones were also streaked on BMM/S plates to isolate single colonies. BMM/S shake flask cultures were inoculated from the patch, grown at 30° C. for 3 days, and analyzed by ELISA for quantification of secreted transferrin fusion protein. Addition of an IgG linker increased productivity in shake flasks for all constructs (Table 8), with the greatest productivity observed for the PEAPTD IgG linker (pREX0558). This construct exhibited an approximately 7-fold increase in GLP-1 transferrin relative to a construct with no linker (pREX0505). Addition of the CEx linker alone (pREX0559) did not significantly increase productivity (Table 8).

TABLE 8
Concentration of GLP-1 Transferrin Fusion
Protein in Shake Flask Culture Supernatant

| Construct | Linker | Yield (μg/μL) |
|---|---|---|
| pREX0556 | IgG hinge | 4.0 ± 0.8 |
| pREX0557 | CEx IgG hinge | 4.8 ± 0.3 |
| pREX0558 | PEAPTD IgG hinge | 7.5 ± 1.1 |
| pREX0559 | Cex | 1.1 ± 0.1 |
| pREX0505 | No linker | 1.0 ± 0.2 |

Protein levels were determined by ELISA. Means represent 3-6 cultures per construct and are followed by the standard error.

A single celled stock was made for constructs pREX0556-559 (Y0389, Y0384, Y0385 and Y0380, respectively) and DNA sequenced to confirm identity. Two fermentations (F0187 and F0190) were run for the IgG construct (pREX0556) with densitometry of an SDS-PAGE gel estimating yields of 679 and 719 μg/mL. Fermentation F0191 of the PEAPTD IgG construct (pREX0558) yielded 960 μg/mL by densitometry. Potency of the GLP-1 fusions was determined by cAMP assays. The EC₅₀ values for F0187 and F0190 (IgG) were 6.6 nM and 8.6 nM, while the EC₅₀ for F0191 (PEAPTD IgG) was 5.9 nM.

ELISA Data

Table 9 below summarizes the ELISA data obtained with the constructs containing GLP-1 and various linkers.

TABLE 9
Summary of ELISA Data From Shake Flask Culture.

| Construct | Linker | μg/mL |
|---|---|---|
| pREX0456 | S | 0.28 |
| pREX0452 | SS | 0.23 |
| pREX0458 | SS | 2.2 |
| pREX0454 | SSG | 2.0 |
| pREX0460 | SSG | 1.7 |
| pREX0518 | PEA | 2.2 |
| pREX0520 | PEAPTD | 4.8 |
| pREX0567 | PEAPTD | 2.1 |
| pREX0569 | PEAPTD | 1.8 |
| pREX0571 | PEAPTD | 1.6 |
| pREX0573 | (PEAPTD)₂ | 2.8 |
| pREX0585 | (PEAPTD)₂ | 3.6 |
| pREX0585 | (PEAPTD)₂ | 1.6 |
| pREX0575 | (PEAPTD)3 | 2.8 |
| pREX0562 | IgG hinge | 3.3 |
| pREX0599 | IgG hinge | 3.8 |
| pREX0564 | PEAPTD IgG hinge | 8.1 |
| pREX0601 | PEAPTD IgG hinge | 7.3 |
| pREX0559 | Cex | 1.1 |
| pREX0602 | Cex | 1.6 |
| pREX0563 | CEx IgG hinge | 4.9 |
| pREX0600 | CEx IgG hinge | 4.7 |

Conclusions

1. Although the long flexible linkers, (SGGG)₃ and GLP-2, resulted in poor productivity; the GLP-2 linker was highly potent.

2. Although the short flexible linkers, S, SS, and SSG, yielded no improvement in potency, the S linker resulted in a dramatic drop in productivity whereas the SS and SSG linkers maintained productivity levels.

3. The substantially non-helical linkers yielded the most favorable results. The PE and PEA linker had little effect on either productivity or potency but the PEAPTD linker, and in particular the duplicate linker, (PEAPTD)₂, improved productivity and brought the potency within about 5 to 10 fold of native GLP-1 peptide. The effect of 3 or 4 repeats on potency had not been tested but did not adversely effect productivity.

4. The IgG linker, particularly in conjunction with the PEAPTD linker, resulted in a significant improvement in productivity. It was also found to bring the in vitro potency of the fusion close to that of native GLP-1 peptide even with the G22E mutation.

5. The construct pREX0585 nL GLP-1 (7-37;A8G;K34A) (PEAPTD)₂ mTf yielded the greatest improvement in potency with minimal deviation from the natural sequence.

### Example 3

### Pharmacokinetics of GLP-1 Analog/Linker/mTf Fusion Protein

In this example, a GLP-1 analog/linker/mTf fusion protein is made and an analysis of this pharmacokinetics is performed.

The present invention provides fusion proteins comprising a GLP-1 analog fused to mTf via a linker. In one embodiment, the GLP-1(7-37) analog comprises amino acid substitutions at positions 8 and 34 (correspond to amino acids 2 and 28 of SEQ ID NO.: 6). For example, the GLP-1(7-37) analog contains a glycine substitution for alanine at position 8 and alanine substitution for lysine at position 34. This prevents dipeptidyl peptidase IV cleavage (substitution at residue 8) and a second enzyme cleavage (substitution at residue 34).
GLP-1(7-37;A8G,K34A): hgegtftsdvssylegqaakefiawlvagrg (SEQ ID NO.: 63)
   The GLP1(7-37;A8G,K34A) is fused to mTf through a linker such as (PEAPTD)₂ to increase the productivity of the fusion protein. The amino terminus of GLP-1(7-37;A8G,K34A) is fused to the nL leader sequence.
nL leader sequence: mrlavgallvcavlglcla (SEQ ID NO.: 64)

The complete nucleic acid and amino acid sequences for the fusion protein nL GLP1 (7-37;A8G,K34A) (PEAPTD)₂ mTf (see plasmids pREX0584 and pREX0585) are below (SEQ ID NO.: 65 and 66).

| | |
|---|---|
| 1 | atgaggctcg ccgtgggagc cctgctggtc tgcgccgtcc tggggctgtg |
| | >>........................nL.........................> |
| | m r l a v g a l l v c a v l g l |
| 51 | tctggcgcat ggtgaaggta cttttacttc tgatgtttct tcttatttgg |
| | >.nL.>> |
| | c l a |
| | >>........... GLP-1 (7-37; A8G; K34A) ............> |
| | h g e g t f t s d v s s y l |
| 101 | aaggtcaagc tgctaaagaa tttattgctt ggttggttgc tggtaggggt |
| | >............... GLP-1 (7-37; A8G; K34A) ...............>> |
| | e g q a a k e f i a w l v a g r g |
| 151 | ccagaagctc caactgatcc agaagctcca actgatgtac ctgataaaac |
| | >>...........PEAPTD linker...........>> |
| | p e a p t d p e a p t d |
| | >>....mTf.....> |
| | v p d k |
| 201 | tgtgagatgg tgtgcagtgt cggagcatga ggccactaag tgccagagtt |
| | >........................mTf.........................> |
| | t v r w c a v s e h e a t k c q s |
| 251 | tccgcgacca tatgaaaagc gtcattccat ccgatggtcc cagtgttgct |
| | >........................mTf.........................> |
| | f r d h m k s v i p s d g p s v a |
| 301 | tgtgtgaaga aagcctccta ccttgattgc atcagggcca ttgcggcaaa |
| | >........................mTf.........................> |
| | c v k k a s y l d c i r a i a a |
| 351 | cgaagcggat gctgtgacac tggatgcagg tttggtgtat gatgcttacc |
| | >........................mTf.........................> |
| | n e a d a v t l d a g l v y d a y |
| 401 | tggctcccaa taacctgaag cctgtggtgg cagagttcta tgggtcaaaa |
| | >........................mTf.........................> |
| | l a p n n l k p v v a e f y g s k |
| | BamHI |
| | −+−−−− |
| 451 | gaggatccac agactttcta ttatgctgtt gctgtggtga agaaggatag |
| | >........................mTf.........................> |
| | e d p q t f y y a v a v v k k d |
| 501 | tggcttccag atgaaccagc ttcgaggcaa gaagtcctgc cacacgggtc |
| | >........................mTf.........................> |
| | s g f q m n q l r g k k s c h t g |
| 551 | taggcaggtc cgctgggtgg aacatcccca taggcttact ttactgtgac |
| | >........................mTf.........................> |
| | l g r s a g w n i p i g l l y c d |
| 601 | ttacctgagc cacgtaaacc tcttgagaaa gcagtggcca atttcttctc |
| | >........................mTf.........................> |
| | l p e p r k p l e k a v a n f f |
| 651 | gggcagctgt gccccttgtg cggatgggac ggacttcccc cagctgtgtc |
| | >........................mTf.........................> |
| | s g s c a p c a d g t d f p q l c |
| 701 | aactgtgtcc agggtgtggc tgctccaccc ttaaccaata cttcggctac |
| | >........................mTf.........................> |
| | q l c p g c g c s t l n q y f g y |
| 751 | tcgggagcct tcaagtgtct gaaggatggt gctggggatg tggcctttgt |
| | >........................mTf.........................> |
| | s g a f k c l k d g a g d v a f |
| 801 | caagcactcg actatatttg agaacttggc aaacaaggct gacagggacc |
| | >........................mTf.........................> |
| | v k h s t i f e n l a n k a d r d |
| 851 | agtatgagct gctttgcctg gacaacaccc ggaagccggt agatgaatac |
| | >........................mTf.........................> |
| | q y e l l c l d n t r k p v d e y |
| 901 | aaggactgcc acttggccca ggtcccttct cataccgtcg tggcccgaag |
| | >........................mTf.........................> |
| | k d c h l a q v p s h t v v a r |
| 951 | tatgggcggc aaggaggact tgatctggga gcttctcaac caggcccagg |
| | >........................mTf.........................> |
| | s m g g k e d l i w e l l n q a q |
| | EcoRI |
| | −+−−−−− |
| 1001 | aacattttgg caaagacaaa tcaaaagaat tccaactatt cagctctcct |
| | >........................mTf.........................> |
| | e h f g k d k s k e f q l f s s p |
| 1051 | catgggaagg acctgctgtt taaggactct gcccacgggt ttttaaaagt |
| | >........................mTf.........................> |
| | h g k d l l f k d s a h g f l k |
| 1101 | cccccccagg atggatgcca agatgtacct gggctatgag tatgtcactg |
| | >........................mTf.........................> |
| | v p p r m d a k m y l g y e y v t |
| | AccIII |
| | −+−−−− |
| 1151 | ccatccggaa tctacgggaa ggcacatgcc cagaagcccc aacagatgaa |
| | >........................mTf.........................> |
| | a i r n l r e g t c p e a p t d e |
| 1201 | tgcaagcctg tgaagtggtg tgcgctgagc caccacgaga ggctcaagtg |
| | >........................mTf.........................> |
| | c k p v k w c a l s h h e r l k |
| | HpaI |
| | −−−+−− |
| 1251 | tgatgagtgg agtgttaaca gtgtagggaa aatagagtgt gtatcagcag |
| | >........................mTf.........................> |
| | c d e w s v n s v g k i e c v s a |
| 1301 | agaccaccga agactgcatc gccaagatca tgaatggaga agctgatgcc |
| | >........................mTf.........................> |
| | e t t e d c i a k i m n g e a d a |
| 1351 | atgagcttgg atggagggtt tgtctacata gcgggcaagt gtggtctggt |
| | >........................mTf.........................> |
| | m s l d g g f v y i a g k c g l |
| 1401 | gcctgtcttg gcagaaaact acaataaggc tgataattgt gaggatacac |
| | >........................mTf.........................> |
| | v p v l a e n y n k a d n c e d t |
| 1451 | cagaggcagg gtattttgct gtagcagtgg tgaagaaatc agcttctgac |
| | >........................mTf.........................> |
| | p e a g y f a v a v v k k s a s d |
| 1501 | ctcacctggg acaatctgaa aggcaagaag tcctgccata cggcagttgg |
| | >........................mTf.........................> |
| | l t w d n l k g k k s c h t a v |
| | NcoI |
| | −+−−−− |
| 1551 | cagaaccgct ggctggaaca tccccatggg cctgctctac aataagatca |
| | >........................mTf.........................> |
| | g r t a g w n i p m g l l y n k i |
| | PstI |
| | −−−−−+ |
| 1601 | accactgcag atttgatgaa tttttcagtg aaggttgtgc ccctgggtct |
| | >........................mTf.........................> |
| | n h c r f d e f f s e g c a p g s |
| 1651 | aagaaagact ccagtctctg taagctgtgt atgggctcag gcctaaacct |
| | >........................mTf.........................> |
| | k k d s s l c k l c m g s g l n |
| 1701 | ctgtgaaccc aacaacaaag agggatacta cggctacaca ggcgctttca |
| | >........................mTf.........................> |
| | l c e p n n k e g y y g y t g a f |
| 1751 | ggtgtctggt tgagaaggga gatgtggcct ttgtgaaaca ccagactgtc |
| | >........................mTf.........................> |
| | r c l v e k g d v a f v k h q t v |
| | NcoI |
| | −+−−−− |
| 1801 | ccacagaaca ctgggggaaa aaaccctgat ccatgggcta agaatctgaa |
| | >........................mTf.........................> |
| | p q n t g g k n p d p w a k n l |
| 1851 | tgaaaaagac tatgagttgc tgtgccttga tggtactagg aaacctgtgg |
| | >........................mTf.........................> |
| | n e k d y e l l c l d g t r k p v |
| 1901 | aggagtatgc gaactgccac ctggccagag ccccgaatca cgctgtggtc |
| | >........................mTf.........................> |
| | e e y a n c h l a r a p n h a v v |
| | SphI |
| | −−−−−−+ |
| 1951 | acacggaaag ataaggaagc atgcgtccac aagatattac gtcaacagca |
| | >........................mTf.........................> |
| | t r k d k e a c v h k i l r q q |
| 2001 | gcacctattt ggaagcaacg tagctgactg ctcgggcaac ttttgtttgt |
| | >........................mTf.........................> |
| | q h l f g s n v a d c s g n f c l |
| 2051 | tccggtcgga aaccaaggac cttctgttca gagatgacac agtatgtttg |
| | >........................mTf.........................> |
| | f r s e t k d l l f r d d t v c l |
| 2101 | gccaaacttc atgacagaaa cacatatgaa aaatacttag gagaagaata |
| | >........................mTf.........................> |
| | a k l h d r n t y e k y l g e e |
| | BstEII |
| | −+−−−−− |
| 2151 | tgtcaaggct gttggtaacc tgagaaaatg ctccacctca tcactcctgg |
| | >........................mTf.........................> |
| | y v k a v g n l r k c s t s s l l |
| | SalI |
| | −+−−−−− |
| 2201 | aagcctgcac tttccgtcga ccttaataa |
| | >..........mTf.........>> |
| | e a c t f r r p |

Pharmacokinetics of GLP-1(A8G,K34A)-PEAPTDPEAPTD-mTf

Two Cynomolgous monkeys per group were injected SC or IV with 2250 μg/kg of GLP-1-Tf fusion protein, as determined by A₂₈₀. The fusion protein used was GLP-1(A8G,K34A)-PEAPTDPEAPTD-mTf. Plasma samples were analyzed using a sandwich ELISA specific for GLP-1-Tf, using a monoclonal antibody to GLP-1 to capture the fusion protein and a polyclonal antibody to Tf to detect the bound protein. The plate was coated with Rabbit anti-mouse Fab (1 μg/well) and then washed and blocked with 1% BSA in PBS, after which a Mouse Anti-GLP-1 MAb was added as the capture antibody. The plate was then washed and standard curve or sample diluted in 1% BSA in PBS is added to the plate. After the incubation period the plate was washed and Biotinylated Anti-Human Transferrin Antibody was add as the detecting antibody. The bound antibody was detected with HRP-Streptavidin and a fluorescent substrate with a wash between each step.

For the bioassay, the samples were incubated with CHO cells transfected with the rat GLP-1 receptor. The GLP-1 receptor (GLP-1R) is a membrane-associated G-protein-coupled receptor, and upon ligand binding, adenylyl cyclase is activated, resulting in a concentration-dependent elevation in intracellular cAMP. The cAMP produced by the cells in response to GLP-1-Tf in the plasma was measured by cAMP ELISA. The cAMP produced by the samples was compared to the cAMP produced by known concentrations of GLP-1-Tf. The PK analysis was performed using PK Summit software.

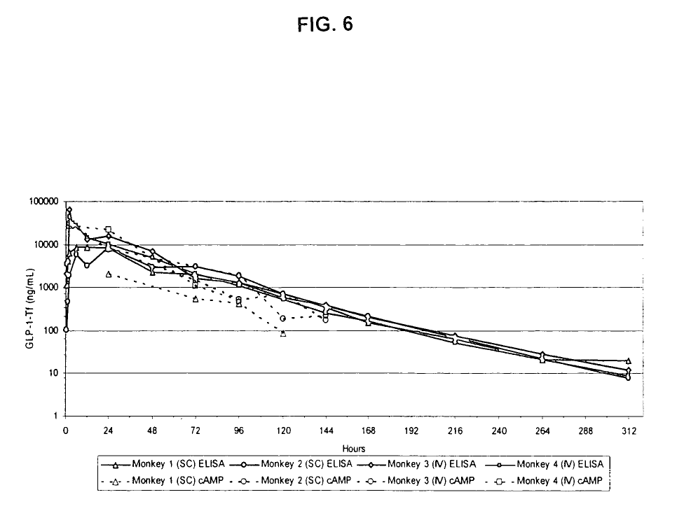

The results are shown in FIG. 6. The terminal half-life in these studies was calculated to be approximately 30 hr. This compares to a half-life of GLP-1 peptide in humans of approximately 90 seconds. In addition, comparison of the area under the curve for the SC and IV routes indicates a high bioavailability via the SC route.

Example 4

### Construction and Use of BRX0585 for Treatment of Diabetes and for Weight Loss

Expression of BRX0585 in Yeast

An expression cassette coding for GLP-1(7-37) attached to the N-terminus of non-glycosylated human transferrin with an intervening PEAPTDPEAPTD linker peptide (SEQ ID NO.: 10) was created. It should be noted that alternative substantially non-helical linkers could have been used such as PEAPTD (SEQ ID NO.: 13), IgG hinge linker (SEQ ID NO.: 88, 89, and 117), IgG hinge linker and PEAPTD (SEQ ID NO.: 118-123), and PEAPTDPEAPTDPEAPTD (SEQ ID NO.: 14).

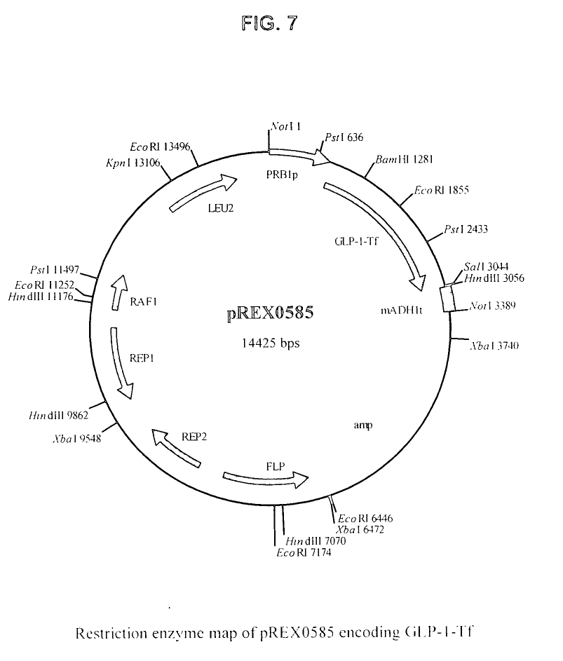

The cassette was designed such that the second amino acid of the GLP-1 moiety was substituted in order to prevent the effect of DPP-IV on the GLP-1 moiety. The transferrin sequence was modified to eliminate the two N-linked glycosylation sites. The cassette was cloned in a high copy number plasmid (see FIG. 7; SEQ ID NO.: 11) and expressed in high levels in Saccharomyces cerevisiae. Similarly, a transferrin sequence with the same modifications used to eliminate the two N-linked glycosylation sites was cloned into a high copy plasmid and expressed in S. cerevisiae. Proteins were purified using several chromatographic steps to >99.9% purity, as judged by SDS-PAGE. SE-HPLC and N-terminal sequencing. The resulting Tf and GLP-1 fusion protein corresponds to the sequence of SEQ ID NO.: 12. The fusion peptide of SEQ ID NO.: 12 is referred to interchangeably in this Example as BRX0585 and GLP-1-Tf.

GLP-1-Tf Activated the GLP-1 Receptor in vitro

Chinese hamster ovary (CHO) cells were stably transfected with the GLP-1 receptor (CHO/GLP-1R cells) and cultured with 5% CO₂ at 37° C. in Ham's F-12 medium (Cellgro) as previously described (Montrose-Rafizadeh et al., 1997, J. Biol. Chem. 272: 21201-21206). CHO/GLP-1R cells were rinsed with Krebs-Ringer buffer (KRB, Cellgro) and incubated for 1 h at 37° C. to lower endogenous intracellular cAMP levels. This was followed by a brief incubation in KRB containing 4 mM IBMX (Sigma) to inhibit intracellular phosphodiesterase that degrade cAMP. Triplicate wells of cells were treated with serial dilutions of GLP-1-Tf, GLP-1 (7-37) or exendin-4 (the latter two from Bachem) for 60 min at 37° C. Afterwards, the supernatants were removed and cells were lysed in 0.1 N HCl. Cell lysates were assayed to determine intracellular cAMP concentrations using a competition-based chemiluminescent enzyme immunoassay (Assay Designs Inc.).

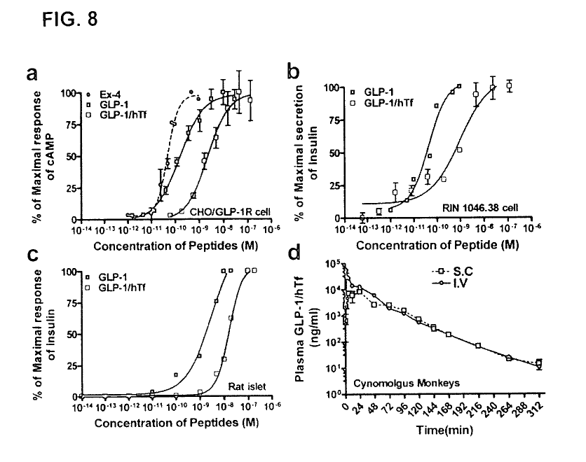

The GLP-1 receptor (GLP-1R) is a membrane-associated G-protein-coupled receptor, and upon ligand binding, adenylyl cyclase is activated, resulting in a concentration-dependent elevation in intracellular cAMP levels. GLP-1-Tf was found to activate adenylyl cyclase in GLP-1R-transfected CHO cells (CHO/GLP-1R). It produced a dose-dependent increase in intracellular cAMP levels and had reduced activity but similar potency compared to native GLP-1 and exendin-4 (EC₅₀: 2.28 vs. 0.16 vs. 0.05 nM, GLP-1-Tf vs. GLP-1 vs. exendin-4, respectively) (FIG. 8a).

Rat insulinoma cell line, RIN1046-38 cells, was obtained from Dr. Samuel A. Clark and cultured with 5% CO₂ at 37° C. in M199 medium (Cellgro) as previously described (Wang et al., 2001, Endocrinology, 142: 1820-1827). RIN 1046-38 cells grown on 12-well plates that had reached 50-60% confluency were washed in glucose-free insulin secretion buffer (Biofluids), and the cells were incubated with the serial dilutions of GLP-1 and GLP-1-Tf for 1 h at 37° C. Exendin-9-39 (1 μmol/l: Bachem), an inhibitor of the GLP-1R. was also added overnight to one set of experiments. The supernatant was then collected and saved at −80° C. for determination of insulin content by ELISA (Crystal Chems). Cells were lysed and the protein content was quantified using the Bradford method.

Islets from Sprague-Dawley rats were isolated as we previously described, with some modification (Wang et al., 1997, J. Clin. Invest. 99: 2883-2889). Briefly, pancreata were digested by Collagenase Type XI (Sigma) and islets separated through a Ficoll-Paque gradient (Amersham Biosciences). The isolated islets were washed several times using Hanks Balanced Salts Solution (Biosource) containing 0.2% bovine serum albumin (BSA, Sigma), hand-picked and cultured overnight with 5% CO₂ at 37° C. in M199 medium supplemented with 5 mM glucose. Rat islets were then treated similarly to (30-40 per well) to RIN cells as described above.

GLP-1-Tf was found to stimulate insulin secretion in both rat insulinoma cells and isolated rat islets in a concentration-dependent manner (FIGS. 8b and c).

Measurement of GLP-1-Tf in Body Fluids

Male cynomolgus monkeys (Macaca fascicularis) (N=4) received intravenous (IV) and subcutaneous (SC) boluses of GLP-1-Tf (2.25 mg/kg) and blood was taken at the times indicated in FIG. 8d. The blood was centrifuged and serum was stored at −80° C. for assay of GLP-1-Tf. All animal experiments were carried out on approved protocols in accordance with the Animal Care & Use Committee of the NIA.

A sandwich ELISA was used to measure the concentration of GLP-1-Tf in body fluids. For this assay, 200 ng of anti-GLP-1 monoclonal antibody (Antibody Shop) was immobilized on microtiter plates coated with anti-mouse IgG. After washing, sample or standard were added to the plate that was then incubated at 37° C. overnight. The plate was next washed and incubated with biotinylated chicken anti-human transferrin antibody (Fitzgerald Laboratories). Washing was repeated and the plate was incubated with horseradish-peroxidase (HRP)-streptavidin. The plate was again washed and the bound HRP was measured with Pierce Quanta Blu Fluorogenic Substrate. The measurements were done using a Spectramax Gemini fluorescence plate reader and Softmax Pro software. Buffer used for all steps was phosphate buffered saline (PBS) containing 1% BSA and 0.05% Tween 20.

The t₁₋₂ of GLP-1-Tf in serum, after both routes of administration, was about 44 hours and the comparison of the SC and IV profile showed essentially 100% bioavailability (FIG. 8d). Further, by treating CHO/GLP-1R with the serum from the injected monkeys, it was found that the GLP-1-Tf measured by ELISA was biologically active and the slope of decay of intracellular cAMP accumulation in response to the serum over time was similar to that of GLP-1-Tf in the ELISA (data not shown).

Glucose Homeostasis and Plasma Insulin Determinations in Non-Diabetic and Diabetic Mice

Male diabetic db/db mice (C57BLKS/J-Lepr^{db}/Lepr^{db}) lacking a functioning leptin receptor and their non-diabetic heterozygous littermates were purchased at 4 weeks of age from Jackson Laboratories (Bar Harbor). They were housed, two per cage and fed ad libitum for at least five days before any testing. The same mice were caged together for the duration of the studies.

Diabetic db/db mice are homozygous for a mutated non-functioning leptin receptor and consequently they have hyperphagia, eat throughout the 24 hour day (instead of the more usual feeding during the dark hours), become obese and by 4-6 weeks of age have elevated blood glucose levels in the 300-400 mg/dl range (normal fasting blood glucose levels are 90-120 mg/dl). Their heterozygous littermates (non-diabetic mice) are not hyperphagic, are lean and do not develop diabetes.

Intraperitoneal (IP) glucose tolerance testing (IPGTT) (0.5 g/kg body weight) was carried out after an overnight fast in non-diabetic mice. Tf and GLP-1-Tf were administered IP 30 min before the glucose injection. Blood glucose levels (Glucometer Elite, Bayer) were measured at 0, and 30 and 60 min and plasma insulin levels at 0 and 30 min after the IPGTT.

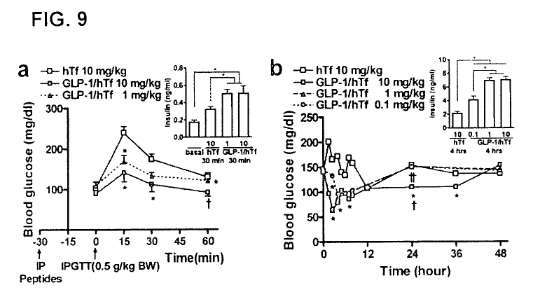

IP administration of 1 and 10 mg/kg GLP-1-Tf resulted in enhanced insulin secretion in the non-diabetic mice. The peak blood glucose was decreased, coinciding with elevated insulin levels at 30 minutes (FIG. 9a).

Tf and GLP-1-Tf were administered subcutaneously to non-diabetic and diabetic mice and blood glucose checked frequently for 48 h for prolonged effects. Plasma insulin levels were measured at 1 and 4 hours. An IPGTT (0.5 g/kg body weight) was also performed after an overnight 12 hour fast in diabetic mice that had received GLP-1-Tf or Tf subcutaneously at the beginning of the fast.

SC administration of GLP-1-Tf dose-dependently reduced blood glucose concentration and increased insulin secretion in ad libitum fed non-diabetic mice (FIG. 9b). Blood glucose levels dropped to 70 mg/dl within 3 hours of administration and gradually rose to the pre-administration levels within 24 hours. Low blood glucose persisted for up to 36 hours with the higher dose of GLP-1-Tf (10 mg/kg).

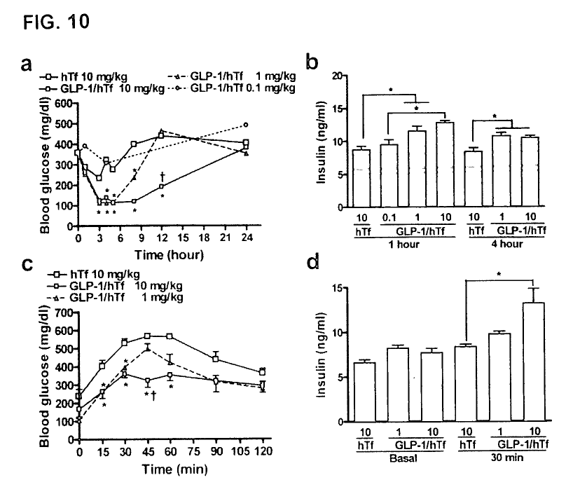

SC administration of GLP-1-Tf (both 1 and 10 mg/kg) louvered blood glucose to normal levels (from 358±23 to 115±18 mg/dl) in diabetic ad libitum mice, the effect beginning as early as 1 hour after injection (FIG. 10a). Increased plasma insulin levels were present for at least up to 4 hours after both 1 and 10 mg/kg GLP-1-Tf (FIG. 10b). The highest dose sustained the effect on blood glucose for at least 12 hours without causing hypoglycemia. A low dose of GLP-1-Tf (0.1 mg/kg) caused an acute, unsustained effect of lowering blood glucose for 4 hours after administration, concomitant with increased plasma insulin levels at 1 hour (FIGS. 10a and b).

A group of diabetic mice were given Tf or GLP-1-Tf via SC administration at 9 pm, fasted overnight and an IPGTT performed the next morning. The fasted Tf-treated animals had lower fasting blood glucose than ad libitum fed animals (238±39 versus 358±23 mg/dl; compare 0 time/fasted animals of FIG. 10c with 0 time/fed animals of FIG. 10a), and additionally, the animals given 1 or 10 mg GLP-1-Tf had lower fasting blood glucose (132±27 mg/dl) than those given Tf alone. After the IPGTT, the animals given GLP-1-Tf had lower glucose levels and higher plasma insulin levels as compared to hTf-treated animals (FIGS. 10c and d).

Food Intake and Blood Glucose Measurements after BRX0585 Administration in Non-Diabetic and db/db Mice

Male diabetic db/db mice (C57BLKS/J-Lepr^{db}/Lepr^{db}) lacking a functioning leptin receptor and their non-diabetic heterozygous littermates were purchased at 4 weeks of age from Jackson Laboratories (Bar Harbor).

Mice (N=5 per group) were conditioned to eat once daily (9-10 am) for five days. On the morning of the sixth day they received IP Tf (10 mg/kg) or GLP-1-TF (0.1, 1 and 10 mg/kg) or exendin-4 (1 nmol/kg) and then allowed to eat ad libitum. Food intake and blood glucose levels were measured 2, 4, 7 and 24 h after peptide administration.

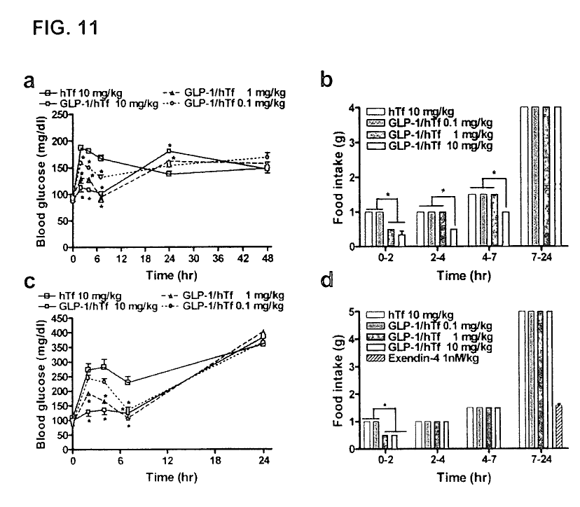

During the five day period, normal fasting glucose levels were maintained in the db/db mice (99±9 mg/dl) and the levels were similar to non-diabetic animals (87±2 mg/dl) (FIG. 11c. 0 h, compared to FIG. 11a. 0 h). Both sets of animals ate approximately 0.5 gram of food pellet during the feeding hour. Two animals were sacrificed on the morning of the sixth day and no food was present in stomach or small bowel. The remaining animals were administered GLP-1-Tf(0.1, 1 and 10 mg/kg). Tf(10 mg/kg) or exendin-4 (1 nmol/kg to the diabetic mice only) before having free access to food. Food intake was quantified and blood glucose levels (in order to measure post-prandial glucose elevations) were measured at intervals for the following 24 h. In non-diabetic animals food intake was decreased by approximately half for up to 7 h by GLP-1-Tf (10 mg/kg) and for 2 h by GLP-1-Tf (1 mg/kg) and post-prandial blood glucose levels were lower, in a concentration-dependent manner, with all three doses, compared to animals that received Tf only (FIG. 11a and b) reflecting both decreased food intake and increased insulin secretion (see FIG. 10b). In the time segment 7-24 h after injection, GLP-1-Tf-treated animals had similar food intake to Tf-treated animals. In db/db animals, food intake was also decreased for 2 hours, by approximately half, with GLP-1-Tf (1 and 10 mg/kg) and all three doses, again in a concentration-dependent manner, lowered post-prandial blood glucose levels, compared to Tf treatment. Exendin-4 essentially prevented all measurable food consumption for 7 h, and caused a decrease in food intake for the remaining 17 h.

Analysis of β-Cell Proliferation and β-Cell Mass

Male diabetic db/db mice (C57BLKS/J-Lepr^{db}/Lepr^{db}) lacking a functioning leptin receptor were purchased at 4 weeks of age from Jackson Laboratories (Bar Harbor). They were housed, two per cage and fed ad libitum for at least five days before any testing. The same mice were caged together for the duration of the studies.

db/db mice, 6 per time point, received one IP injection of Tf (10 mg/kg) or GLP-1-Tf (10 mg/kg) and were sacrificed 1, 2 and 3 days later for measurement of β-cell proliferation and 3, 5, 8 and 10 days later for measurement of islet mass. Sixty mg/kg 5-bromo-2-deoxyuridine (BrdU, Sigma) was injected IP 6 hours before sacrifice. BrdU is a thymidine analog that is incorporated during S-phase of cell mitosis. Pancreata were removed, fixed overnight in 4% buffered formalin, processed for embedding in paraffin and histological sections (4 μm) mounted on poly-1-lysine-coated glass slides. BrdU was detected with a BrdU antibody (Sigma, 1:500 and also confirmed with Zymed antibody, 1:250) and BrdU staining kit (Zymed Laboratories Inc). Five to six hundred islets from each condition were then visualized and the number of islets that contained BrdU+ cells as well as the percent BrdU+ nuclei per total number of islet cells was quantified. For insulin staining and determination of β-cell mass histological sections were treated with a guinea-pig insulin antibody (Linco). Antibody binding was visualized with 3,3-diaminobenzidine (DAB) and sections counterstained with hematoxylin (Vectastain ABC kit, Vector Laboratories, using goat anti-guinea pig IgG). The sections were examined using a video camera connected to a computer with imaging software. Sectioned tissue images were acquired through a 2.5× objective of a phase contrast light microscope (Carl Zeiss) and digitized by means of a Sony Power HAD digital camera (an average of 20-30 images per section). The total pancreatic area and β-cell positive area for every image were quantified using MetaMorph 4.6.3 software (Universal Imaging Inc., West Chester).

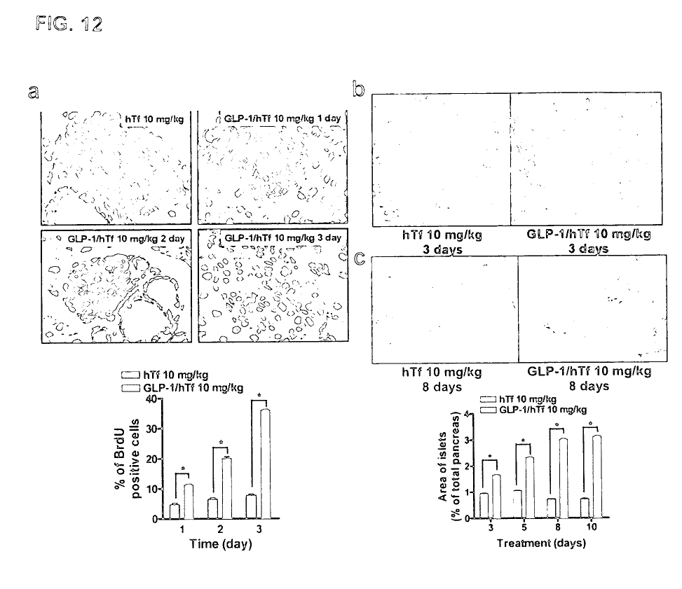

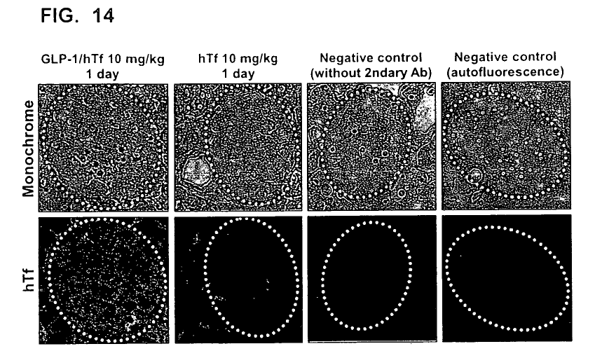

The GLP-1-Tf-treated animals had a clear increase in the number of BrdU+ nuclei in islets as early as 24 hours after GLP-1-Tf injection, compared to Tf treatment only. By the third day after active treatment, there was a 4.7-fold increase in BRDU+ nuclei (p<0.01) (FIG. 12a) and all islets in GLP-1-Tf animals had 1 or more BRDU+ nuclei, compared to 35% of islets (140 out of 400) of hTf-treated animals. There was an occasional islet in GLP-1-Tf-treated animals where BRDU+ nuclei were seen in as many as 50% of the islet cell nuclei (FIG. 12b). The BRDU+ nuclei were mainly in insulin-containing β-cells (FIG. 12b). After 3 days of treatment with GLP-1-Tf(10 mg/kg), total β-cell mass was increased 1.7-fold, compared to Tf treatment, and by 5, 8 and 10 days, total β-cell mass was increased even further, by 2.2-, 4.1-, and 4.3-fold, respectively (FIG. 12c). Using immunofluorescence and confocal microscopy, we could detect the presence of GLP-1-Tf for up to 24 h in islets from fixed pancrcata that had been taken from Tf- and GLP-1-Tf-treated mice (FIG. 14).

Determination of Cerebrospinal Fluid (CSF) Levels of hTf and GLP-1-Tf

Male Sprague Dawley rats were purchased at 6 weeks of age from Harlan Sprague Dawley (Indianapolis). Rats were injected IP with hTf and GLP-1-Tf. Animals were sacrificed 2 and 24 h later for analysis of the presence of the proteins in plasma, CSF and brain. CSF was removed by insertion of a 30 gauge needle with syringe attached between the atlas and axis and the CSF gently aspirated. CSF used for analysis was without blood contamination. Rats were used in this experiment in order to obtain sufficient quantity of CSF. The brains were removed, sliced, sonicated in SDS lysis buffer and the homogenate stored at −80° C. for later subjection to western blot analysis.

Plasma samples (0.1 μl) or 5 μl of CSF samples and brain homogenates were mixed with 10 μl SDS-PAGE sample buffer and were subjected to 10% SDS-PAGE (Novex) according to the supplier protocol. The proteins were transferred onto PVDF membrane (BioRad) in 25 mM Tris+192 mM Glycine. The filter paper was equilibrated with 25 mM borate buffer pH 8.0+150 mM NaCl+5% non-fat dry milk. Rabbit anti-Tf (Rockland Immunochemicals) was added to the blot and was incubated overnight at room temperature. After extensive washing, HRP-conjugated goat anti-rabbit IgG (Pierce) was used as a secondary antibody. The bound antibodies to Tf and GLP-1-Tf bands were detected by SuperSignal West Pico chemiluminescent detection kit (Pierce).

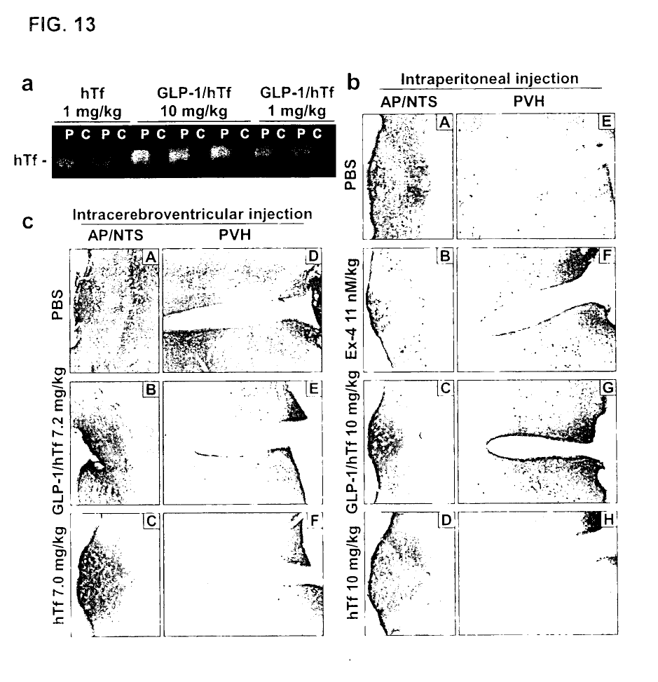

Western blotting of plasma with anti-Tf gave the expected 77 kDa size representing the full-length size, no other fragments were seen and a concentration-dependency was evident (FIG. 13a). In agreement with the ELISA, no immunoreactive Tf was seen in CSF by western blot analysis. We also incubated CHO/GLP-1R cells with CSF taken after 2 h from the GLP-1-Tf-treated rats and could not detect any increase in intracellular cAMP as a result and, in addition, homogenized brain slices from the animals did not contain Tf by western blot analysis. Therefore, it was concluded that GLP-1-Tf does not cross the blood-brain-barrier.

c-Fos Activation in Brain

The pattern of c-Fos expression (used as a marker of activation of neurons) in the brain of mice was examined, in order to determine if peripherally administered GLP-1-Tf was capable of activating the central nervous system and those results were compared to results obtained with IP exendin-4 (1 nmol/kg=4 μg/kg), which is known to activate certain brain areas when given peripherally (Baggio et al., 2004, Gastroenterology. 127: 546-558).

Briefly, mice that received IP injections were anesthetized with isoflurane 2 h later and the mice that received ICV injections were anesthetized 1.5 and 24 h later. Intracardiac perfusion with ice-cold 4% paraformaldehyde was immediately carried out. Brains were removed immediately at the end of perfusion, kept in ice-cold 4% parafor maldehyde for 72 h, and then transferred to a solution containing 4% paraformaldehyde and 25% sucrose until they floated. Each brain as cut into 30 μm sections using a sliding microtome with a freezing stage. Brain sections from each animal were placed in a 12-well plate and then processed for immunocytochemistry. The free-floating sections from each animal were first incubated in 0.3% H₂O₂ for 30 min and then washed 3 times in PBS for 5 min/rinse. Thereafter the sections were incubated in blocking serum (normal goat serum/PBS/0.1% Triton) for 45 min at room temperature. The sections were stained for c-Fos with an antibody directed against the amino acid residues 4-17 in rabbit anti-human c-Fos (Calbiochem, 1:30,000) for 48 h at 4° C. The sections were then washed 6 times with PBS/0.1% Triton before incubation for 1.5 h at room temperature with biotinylated goat anti-rabbit IgG (1:600) in blocking serum (Vector Laboratories). Thereafter the sections were visualized with DAB (Vectastain ABC kit) and developed until the color reached the required intensity in the control sections. The reaction was stopped by immersion of the slides in distilled water. After the staining was completed, sections were mounted on superfrost slides (Fisher Scientific), air-dried for 1-2 days and coverslipped with Permount. Sections were examined using light microscopy to identify c-Fos-positive cells.

Peripheral administration of GLP-1-Tf(10 mg/kg dose) activated c-Fos in neurons of the area postrema (AP) (4/6 animals), the nuclei of the solitary tract (NTS) (6/6 animals) and the paraventricular nuclei of the hypothalamus (PVH) (6/6 animals) 2 h after administration (FIG. 13b). GLP-1-Tf (1 mg/kg) activated NTS (6/6 animals) and PVH (6/6 animals), but not AP (0/6 animals) (data not shown). Exendin-4 activated similar areas to GLP-1-Tf in all injected animals, but the AP response was much more robust compared to GLP-1-Tf (FIG. 13b).

In another series of experiments, Tf and GLP-1-Tf were administered intracerebrovenitricular (ICV) and c-Fos activation assessed 1.5 and 24 hours later. Ninety minutes after ICV injection of GLP-1-Tf, c-Fos activation had occurred in PVH and NTS but not AP (FIG. 13c). c-Fos activation was not present in any of the three brain regions 24 h after ICV injection (data not shown).

Statistical Methods

All results are given as means±SE. Student's test was based on the results of the F test that assessed the equality of variance of the two means. If the variances were statistically significantly different then the t test was based on unequal variances. An ANOVA test was used to calculate the significance of difference between more than 2 samples, followed by followed by post hoc testing with Scheffé's test. P values <0.05 were considered statistically significant.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

What is claimed:

1. A fusion protein comprising a GLP-1 peptide, a substantially non-helical polypeptide linker, and a modified transferrin (mTf) molecule exhibiting reduced glycosylation as compared to the native transferrin molecule.

2. A fusion protein of claim 1, wherein the linker is selected from the group consisting of PEAPTD, (PEAPTD)₂, PEAPTD in combination with an IgG hinge linker, and (PEAPTD)₂ in combination with an IgG hinge linker.

3. A fusion protein of claim 1, wherein the linker substantially increases the productivity of expression of the fusion protein as compared to a fusion protein without the rigid linker or the fusion protein with a flexible polypeptide linker.

4. A fusion protein of claim 1, wherein the linker substantially increases the potency of the GLP-1 peptide as compared to a fusion protein without the rigid linker or the fusion protein with a flexible polypeptide linker.

5. A fusion protein of claim 1, wherein the GLP-1 peptide exhibits extended serum half-life as compared to the GLP-1 molecule in an unfused state.

6. A fusion protein of claim 1, wherein the GLP-1 peptide exhibits prolonged biological activity as compared to the GLP-1 molecule in an unfused state.

7. A fusion protein of claim 1, wherein the GLP-1 peptide is at the N-terminus of the fusion protein, at the C-terminus of the fusion protein or at both the N- and C-terminus of the fusion protein.

8. A fusion protein of claim 1, comprising at least two GLP-1 peptides.

9. A fusion protein of claim 1, wherein the GLP-1 peptide has been modified to have substantially reduced protease cleavage.

10. A fusion protein of claim 1, wherein the N-terminus of the fusion protein comprises a secretion signal sequence prior to cleavage.

11. A fusion protein of claim 10, wherein the signal sequence is a signal sequence from serum transferrin, lactoferrin, melanotransferrin, or a variant thereof.

12. A fusion protein of claim 10, wherein the signal sequence is a HSA/MFα-1 hybrid leader sequence or a Tf signal sequence.

13. A fusion protein of claim 11, wherein the signal sequence is the Tf signal sequence comprising amino acids 1-19 of SEQ ID NO: 2.

14. A fusion protein of claim 1, wherein the GLP-1 (7-37) (SEQ ID NO.: 6) has been modified.

15. A fusion protein of claim 14, wherein GLP-1 (7-37) has been modified by mutating A8 to S, G, or V (A2 of SEQ ID NO. 6 to S, G, or V).

16. A fusion protein of claim 14, wherein GLP-1 (7-37) has been modified by mutating K34 to Q, A, or N (K28 of SEQ ID NO.: 6 to Q, A, or N).

17. A fusion protein of claim 14, wherein GLP-1 (7-37) has been modified to delete V33 to G37 (V27 to G31 of SEQ ID NO.: 6).

18. A fusion protein of claim 14, wherein GLP-1 has been modified to delete A30 to G37 (A24 to G31 of SEQ ID NO. 6).

19. A fusion protein of claim 14, wherein the modified GLP-1 is GLP-1(7-37; A8G,K34A).

20. A fusion protein of claim 1, wherein the mTf molecule has reduced affinity for a transferrin receptor (TfR).

21. A fusion protein of claim 20, wherein the mTf molecule does not substantially cross the blood brain barrier.

22. A fusion protein of claim 1, wherein the mTf molecule is modified lactoferrin or modified melanotransferrin.

23. A fusion protein of claim 1, wherein the mTf protein has reduced affinity for iron.

24. A fusion protein of claim 23, wherein the mTf protein does not bind iron.

25. A fusion protein of claim 1, wherein the mTf protein exhibits no N-linked glycosylation.

26. A fusion protein of claim 1, wherein the mTf protein exhibits no glycosylation.

27. A fusion protein of claim 1, wherein said mTf protein comprises at least one mutation that prevents glycosylation.

28. A fusion protein of claim 27, wherein the mutation is within or adjacent to an N-linked glycosylation site comprising the sequence N-X-S/T.

29. A fusion protein of claim 28, wherein the N-X-S/T site corresponds to amino acid N413 or N611 of SEQ ID NO: 3.

30. A fusion protein of claim 29, wherein the mutation is within an N-linked glycosylation site at both N413 and N611 of SEQ ID NO: 3.

31. A fusion protein of claim 1, wherein the fusion protein comprises SEQ ID NO: 12.

32. A nucleic acid molecule encoding a fusion protein of claim 1.

33. A vector comprising a nucleic acid molecule of claim 32.

34. A host cell comprising a vector of claim 33.

35. A host cell comprising a nucleic acid molecule of claim 32.

36. A method of expressing a fusion protein comprising culturing a host cell of claim 34 under conditions which express the encoded fusion protein.

37. A method of expressing a fusion protein comprising culturing a host cell of claim 35 under conditions which express the encoded fusion protein.

38. A host cell of claim 34, wherein the cell is prokaryotic or eukaryotic.

39. A host cell of claim 35, wherein the cell is prokaryotic or eukaryotic.

40. A host cell of claim 38, wherein the cell is a yeast cell.

41. A host cell of claim 39, wherein the cell is a yeast cell.

42. A non-human transgenic animal comprising a nucleic acid molecule of 32.

43. A method of producing a fusion protein comprising isolating a fusion protein from a transgenic animal of claim 42.

44. A pharmaceutical composition comprising the fusion protein of claim 1 and a carrier.

45. A method of treating a subject comprising administering to the subject a therapeutically effective amount of a fusion protein of claim 1.

46. A method of claim 45, wherein the subject is suffering from elevated levels of glucose as compared to a healthy subject.

47. A method of claim 46, wherein the elevated glucose level is associated with diabetes.

48. A method of claim 47, wherein the diabetes is Type II diabetes.

49. A method of regulating glucose levels in a subject comprising administering to the subject a therapeutically effective amount of a fusion protein of claim 1.

50. A method of claim 48, wherein the fusion protein is administered in combination with one or more agents selected from the group consisting of metformin, a DPPIV inhibitor, an NEP 24.11 inhibitor and a glitazone or derivative thereof.

51. A method of decreasing food intake in an animal, comprising administering an effective amount of a fusion protein of claim 1.

52. A method of inducing a cell β proliferation or β-cell mass increase in a patient, comprising administering an effective amount of a fusion protein of claim 1.

53. A method of inducing insulin secretion in a patient in need thereof, comprising administering an effective amount of a fusion protein of claim 1.

54. A method of treating type I diabetes in a patient, comprising administering an effective amount of a fusion protein of claim 1.

55. A method of decreasing gastric emptying in an animal, comprising administering an effective amount of a fusion protein of claim 1.

56. A method of inducing weight loss in an animal, comprising administering an effective amount of a fusion protein of claim 1.

57. A method of treating congestive heart failure in a patient, comprising administering an effective amount of a fusion protein of claim 1.

58. A method of treating non-alcoholic, non-fatty liver disease in a patient, comprising administering an effective amount of a fusion protein of claim 1.

59. A fusion protein comprising a GLP-1 peptide, a substantially non-helical polypeptide linker and a transferrin molecule.
